(19) United States
(12) Patent Application Publication
(10) Pub. No.: US 2020/0317803 A1
(43) Pub. Date: Oct. 8, 2020

(54) ANTI-CD73, ANTI-PD-L1 ANTIBODIES AND CHEMOTHERAPY FOR TREATING TUMORS

(71) Applicant: MEDIMMUNE, LLC, GAITHERSBURG, MD (US)

(72) Inventors: Rakesh KUMAR, GAITHERSBURG, MD (US); Zachary COOPER, GAITHERSBURG, MD (US); Anis KHAN, GAITHERSBURG, MD (US); Judson ENGLERT, GAITHERSBURG, MD (US); Nancy Kathryn MUELLER, Gaithersburg, MD (US); Charles FERTÉ, GAITHERSBURG, MD (US); Pablo MARTINEZ RODRIGUEZ, GAITHERSBURG, MD (US)

(21) Appl. No.: 16/837,477

(22) Filed: Apr. 1, 2020

Related U.S. Application Data
(60) Provisional application No. 62/828,177, filed on Apr. 2, 2019.

(51) Int. Cl. C07K 16/28; A61K 31/555; A61K 31/519; A61K 31/513; C07K 16/22; A61K 31/337; A61K 33/24; A61K 31/7068; A61P 35/00

(52) U.S. Cl. CPC C07K 16/2896; C07K 16/2827; A61K 31/555; A61K 31/519; A61K 31/513; A61K 2039/54; A61K 31/337; A61K 33/24; A61K 31/7068; A61P 35/00; C07K 16/22

(57) ABSTRACT

This disclosure relates to a monoclonal antibody directed against CD73 or an antigen-binding fragment thereof, and the use of such antibody or antigen-binding fragment thereof in the treatment of tumors. The disclosure also relates to methods for the treatment of tumors comprising administering to a patient in need thereof an anti-CD73 antibody or antigen-binding fragment thereof in combination with a monoclonal antibody directed against programmed death-ligand 1 (PD-L1) also known as B7 homolog 1 (B7-H1), or an antigen-binding fragment thereof. The disclosure also relates to methods for the treatment of tumors comprising administering to a patient in need thereof an anti-CD73 antibody or antigen-binding fragment thereof in combination with a PD-L1 antibody or an antigen-binding fragment thereof and chemotherapy.

### CROSS-REFERENCE TO RELATED APPLICATIONS

This application claims to the benefit of U.S. Patent Application Ser. No. 62/828,177, filed Apr. 2, 2019, the entire contents of which is incorporated by reference.

### FIELD OF THE INVENTION

This disclosure relates to a monoclonal antibody directed against anti-cluster differentiation [CD]73 (CD73) or an antigen-binding fragment thereof, and the use of such antibody or antigen-binding fragment thereof in the treatment of tumors. The disclosure also relates to methods for the treatment of tumors comprising administering to a patient in need thereof an anti-CD73 antibody or antigen-binding fragment thereof in combination with a monoclonal antibody directed against programmed death-ligand 1 (PD-L1) also known as B7 homolog 1 (B7-H1), or an antigen-binding fragment thereof. The disclosure also relates to methods for the treatment of tumors comprising administering to a patient in need thereof an anti-CD73 antibody or antigen-binding fragment thereof in combination with a PD-L1 antibody or an antigen-binding fragment thereof and/or chemotherapy.

### BACKGROUND

CD73 or ecto-5′-nucleotidase (5′-NT) is ubiquitously expressed in a number of tissues. This protein is anchored to the cell membrane through a glycosylphosphatidylinositol (GPI) linkage, has ecto-enzyme activity, and plays a role in signal transduction. The primary function of CD73 is the conversion of extracellular nucleotides (e.g., 5′-AMP), to which cells are generally impermeable, to their corresponding nucleosides (e.g., adenosine), which can readily enter most cells. CD73 production of adenosine by the dephosphorylation of AMP, has been shown to regulate adenosine receptor engagement in many tissues, indicating that adenosine functions in cytoprotection, cell growth, angiogenesis and immunosuppression, and also plays a role in tumorigenesis.

CD73 expression on tumor cells has been reported in several types of cancer, including colorectal cancer, pancreatic cancer, bladder cancer, leukemia, lymphoma, glioma, glioblastoma, melanoma, ovarian cancer, thyroid cancer, esophageal cancer, prostate cancer, and breast cancer. Elevated CD73 expression has also been associated with tumor invasiveness, metastasis, and reduced patient survival time. CD73 generates an immunosuppressed environment, characterized by increased adenosine levels, which promote the development and progression of cancer. Notably, CD73 expression has been associated with a prometastatic phenotype in melanoma and breast cancer.

Programmed death-ligand 1 (PD-L1), also known as B7H1, is a 40 kDa transmembrane protein that is a major obstacle in anti-cancer immunity. PD-L1 binding to the programmed death receptor (PD-1), inactivates T-cells, protects tumor cells, and suppresses immune system detection, allowing for unchecked proliferation of cancer cells. PD-L1 also binds CD80, a co-stimulatory molecule. A wide range of tumorigenic and activated immune cell types naturally express PD-L1, including antigen presenting cells, macrophages, monocytes, B cells, T cells and non-hematopoietic cells. Further, inflammatory cytokines induce PD-L1 expression; including interferon gamma (IFNγ). Activated T-cells produced IFNγ, the most potent inducer of PD-L1. IFNγ in turn induces PD-L1 expression, promoting tumor protection, a mechanism known as adaptive immune resistance.

Immune-checkpoint inhibitors hold great potential as cancer therapeutics. Nevertheless, clinical benefits from immune-checkpoint inhibition have been modest. One potential explanation is that tumors use nonoverlapping immunosuppressive mechanisms to facilitate immune escape. Accordingly, improved compositions and methods for reducing tumor-mediated immunosuppression are required.

### SUMMARY

The disclosure provides a method of treating a tumor in a human patient, comprising administering oleclumab or antigen-binding fragment thereof to the patient. In particular embodiments, oleclumab or antigen-binding fragment thereof is administered at a dose of 750-3000 mg. In particular embodiments, oleclumab or antigen-binding fragment thereof is administered at a dose of 40 mg/kg.

The disclosure further provides a method of treating a tumor in a human patient, comprising administering oleclumab or antigen-binding fragment thereof and durvalumab or antigen-binding fragment thereof to the patient.

The disclosure further provides a method of treating a tumor in a human patient, comprising administering oleclumab or antigen-binding fragment thereof and chemotherapy to the patient.

In some aspects, a method of treating a tumor in a human patient comprises administering oleclumab or antigen-binding fragment thereof to the patient.

In some aspects, a method of treating a tumor in a human patient comprises administering oleclumab or antigen-binding fragment thereof and durvalumab or antigen-binding fragment thereof to the patient.

In some aspects, a method treating a tumor in a human patient comprises administering oleclumab or antigen-binding fragment thereof and chemotherapy to the patient. In some aspects, the method further comprises administering durvalumab or antigen-binding fragment thereof.

In some aspects, the oleclumab or antigen-binding fragment thereof is administered at a dose of 750 mg to 3000 mg. In some aspects, the oleclumab or antigen-binding fragment thereof is administered at a dose of 750 mg. In some aspects, the oleclumab or antigen-binding fragment thereof is administered at a dose of 1500 mg. In some aspects, the oleclumab or antigen-binding fragment thereof is administered at a dose of 2250 mg. In some aspects, the oleclumab or antigen-binding fragment thereof is administered at a dose of 3000 mg. In some aspects, the oleclumab or antigen-binding fragment thereof is administered at a dose of 2250 mg and then at a dose of 3000 mg. In some aspects, the oleclumab or antigen-binding fragment thereof is administered at a dose of 2250 mg for four doses and then at a dose of 3000 mg.

In some aspects, the oleclumab or antigen-binding fragment thereof is administered every 14 to 28 days. In some aspects, the oleclumab or antigen-binding fragment thereof is administered every 14 days. In some aspects, the oleclumab or antigen-binding fragment thereof is administered every 28 days. In some aspects, the oleclumab or antigen-binding fragment thereof is administered every 14 days for at least two doses and then every 28 days. In some aspects, the oleclumab or antigen-binding fragment thereof is administered every 14 days for four doses and then every 28 days. In some aspects, the oleclumab or antigen-binding fragment thereof is administered every 21 days. In some aspects, the oleclumab or antigen-binding fragment thereof is administered every 21 days for at least two doses and then every 28 days. In some aspects, the oleclumab or antigen-binding fragment thereof is administered every 21 days for two to four doses and then every 28 days. In some aspects, the oleclumab or antigen-binding fragment thereof is administered every 21 days for two doses and then once every 28 days. In some aspects, the oleclumab or antigen-binding fragment thereof is administered once every 21 days for four doses and then once every 28 days.

In some aspects, the oleclumab or antigen-binding fragment thereof is administered at a dose of 2250 mg once every 21 days for two doses and then at a dose of 3000 mg once every 28 days. In some aspects, the oleclumab or antigen-binding fragment thereof is administered at a dose of 2250 mg once every 21 days for four doses and then at a dose of 3000 mg once every 28 days.

In some aspects, the oleclumab or antigen-binding fragment thereof is administered intravenously.

In some aspects, the durvalumab or antigen-binding fragment thereof is administered at a dose of 1500 mg.

In some aspects, the durvalumab or antigen-binding fragment thereof is administered every 21 days to every 28 days. In some aspects, the durvalumab or antigen-binding fragment thereof is administered every 28 days. In some aspects, the durvalumab or antigen-binding fragment thereof is administered every 21 days. In some aspects, the durvalumab or antigen-binding fragment thereof is administered every 21 days for at least two doses and then every 28 days. In some aspects, the durvalumab or antigen-binding fragment thereof is administered every 21 days for four doses and then every 28 days.

In some aspects, the durvalumab or antigen-binding fragment thereof is administered at a dose of 1500 mg every 21 days for four doses and then at a dose of 1500 mg every 28 days.

In some aspects, the durvalumab or antigen-binding fragment thereof is administered intravenously.

In some aspects, the chemotherapy comprises at least one of cisplatin, pemetrexed. nab-paclitaxel, carboplatin, gemcitabine, cisplatin, oxaliplatin, leucovorin, 5-fluorouracil, and docetaxel.

In some aspects, the chemotherapy comprises oxaliplatin, leucovorin, and 5-fluorouracil.

In some aspects, the oxaliplatin is administered at a dose of 85 mg/m². In some aspects, the oxaliplatin is administered every 2 weeks.

In some aspects, the leucovorin is administered at a dose of 400 mg/m². In some aspects, the leucovorin is administered every 2 weeks.

In some aspects, the 5-fluorouracil is administered at a dose of 2400 mg/m². In some aspects, the 5-fluorouracil is administered by continuous intravenous infusion for 46 to 48 hours. In some aspects, the 5-fluorouracil is administered over 46 to 48 hours every 2 weeks.

In some aspects, the chemotherapy comprises 85 mg/m² oxaliplatin, 400 mg/m² leucovorin and 2400 mg/m² 5-fluorouracil.

In some aspects, the method further comprises administering bevacizumab or an antigen-binding fragment thereof. In some aspects, the bevacizumab or an antigen-binding fragment thereof is administered at a dose of 5 mg/kg. In some aspects, the bevacizumab or an antigen-binding fragment thereof is administered every 2 weeks. In some aspects, the bevacizumab or an antigen-binding fragment thereof is administered intravenously.

In some aspects, the chemotherapy comprises (a) nab-paclitaxel and carboplatin; (b) gemcitabine and cisplatin; (c) gemcitabine and carboplatin; (d) pemetrexed and carboplatin; and (e) pemetrexed and cisplatin.

In some aspects, the nab-paclitaxel is administered at a dose of 100 mg/m². In some aspects, the nab-paclitaxel is administered on days 1, 8, and 15 of a 21-day cycle.

In some aspects, the gemcitabine is administered at a dose of 1000 mg/m² or 1250 mg/m². In some aspects, the gemcitabine is administered on days 1 and 8 of a 21-day cycle.

In some aspects, the pemetrexed is administered at a dose of 500 mg/m². In some aspects, the pemetrexed is administered every three weeks.

In some aspects, the carboplatin is administered at a dose of AUC 5 or 6. In some aspects, the carboplatin is administered every three weeks.

In some aspects, the cisplatin is administered at a dose of 75 mg/m². In some aspects, the cisplatin is administered every three weeks.

In some aspects, the chemotherapy comprises 1000 mg/m² gemcitabine and 125 mg/m² nab-paclitaxel.

In some aspects, the chemotherapy is administered every 7 days to 28 days. In some aspects, the chemotherapy is administered every 14 days.

In some aspects, the administration of oleclumab or antigen-binding fragment thereof results in a partial response. In some aspects, the administration of oleclumab or antigen-binding fragment thereof results in a complete response.

In some aspects, the tumor is a solid tumor. In some aspects, the solid tumor is breast cancer, ovarian cancer, head and neck cancer, prostate cancer, bladder cancer, colorectal cancer, non-small cell lung cancer (NSCLC), glioblastoma, renal cell cancer, or pancreatic cancer. In some aspects, the pancreatic cancer is pancreatic ductal adenocarcinoma. In some aspects, the tumor is a resectable NSCLC tumor. In some aspects, the tumor is an early-stage NSCLC tumor. In some aspects, the tumor is stage IV NSCLC tumor. In some aspects, the colorectal cancer is metastatic microsatellite-stable.

In some aspects, the tumor has high-PD-L1 expression. The tumor with high-PD-L1 expression can be a NSCLC tumor.

In some aspects, the tumor has low-PD-L1 expression. The tumor with low-PD-L1 expression can be a NSCLC tumor.

In some aspects, the tumor lacks an activating epidermal growth factor receptor (EGFR) mutation and/or an anaplastic lymphoma kinase (ALK) fusion. The tumor that lacks an EGFR mutation and/or an ALK fusion can be a NSCLC tumor.

In some aspects, the patient has metastatic pancreatic ductal adenocarcinoma that has not been previously treated. In some aspects, the patient has metastatic pancreatic ductal adenocarcinoma that was previously treated with gemcitabine-based therapy.

In some aspects, the tumor has not received prior treatment in the recurrent and/or metastatic setting. In some aspects, the patient has progressed on an anti-PD-1 or anti-PD-L1 containing therapy.

In some aspects, the tumor is a 1^{st} line metastatic pancreatic ductal adenocarcinoma, wherein the oleclumab or antigen binding fragment thereof is administered at 1500 mg or 3000 mg every 2 weeks for four doses and then every 4 weeks, and wherein the chemotherapy comprises 1000 mg/m² gemcitabine and 125 mg/m² nab-paclitaxel, wherein the chemotherapy is administered on days 1, 8, and 15 of four 28-day cycles and then every 4 weeks.

In some aspects, the tumor is a 2^{nd} line metastatic pancreatic ductal adenocarcinoma, the oleclumab or antigen binding fragment thereof is administered at 1500 mg or 3000 mg every 2 weeks for four doses and then every 4 weeks, and wherein the chemotherapy comprises 85 mg/m² oxaliplatin, 400 mg/m² leucovorin, and 400 mg/m² 5-FU followed by 2400 mg/m² 5-FU, wherein the chemotherapy is administered on days 1 and 15 of four 28-day cycles and then every 4 weeks.

In some aspects, the method further comprises administering 1500 mg durvalumab or an antigen-binding fragment thereof every 4 weeks.

In some aspects, the tumor is a 1^{st} line stage IV NSCLC with high PD-L1 expression, wherein the oleclumab or antigen binding fragment thereof is administered at 1500 mg or 3000 mg every 2 weeks for two 14-day cycles and then every 4 weeks, and wherein the durvalumab or an antigen-binding fragment thereof is administered at 1500 mg every 4 weeks.

In some aspects, the tumor is a 1^{st} line stage IV NSCLC with low PD-L1 expression, wherein the oleclumab or antigen binding fragment thereof is administered (a) at 1500 mg every 3 weeks for four 21-day cycles and then every 4 weeks; or (b) at 2250 mg every 3 weeks for four 21-day cycles and then at 3000 mg every 4 weeks; the durvalumab or antigen binding fragment thereof is administered at 1500 mg every 3 weeks for four 21-day cycles and then every 4 weeks; and the chemotherapy comprises: (a) 100 mg/m² nab-paclitaxel on days 1, 8, and 15 of a 21-day cycle for 4 cycles and 5 or 6 AUC carboplatin on day 1 of the 21-day cycle for 4 cycles; (b) 1000 mg/m² or 1250 mg/m² gemcitabine on days 1 and 8 of a 21-day cycle for 4 cycles and 75 mg/m² cisplatin on day 1 of the 21-day cycle for 4 cycles; (c) 1000 mg/m² or 1250 mg/m² gemcitabine on days 1 and 8 of a 21-day cycle for 4 cycles and 5 or 6 AUC carboplatin on day 1 of the 21-day cycle for 4 cycles; (d) 500 mg/m² pemetrexed on day 1 of 21-day cycle for 4 cycles and 5 or 6 AUC carboplatin on day 1 of the 21-day cycle for 4 cycles, optionally wherein 500 mg/m² pemetrexed is administered every 4 weeks as a maintenance therapy after the 4 cycles; or (e) 500 mg/m² pemetrexed on day 1 of 21-day cycle for 4 cycles and 75 mg/m² cisplatin on day 1 of the 21-day cycle for 4 cycles, optionally wherein 500 mg/m² pemetrexed is administered every 4 weeks as a maintenance therapy after the 4 cycles.

In some aspects, the tumor is a locally advanced, unresectable, stage III NSCLC tumor, and wherein (i) 1500 mg durvalumab or an antigen-binding fragment thereof is administered every 4 weeks and (ii) 3000 mg oleclumab or an antigen-binding fragment thereof is administered every 2 weeks for 2 months and then every 4 weeks.

In some aspects, the tumor is a resectable, early NSCLC tumor, and wherein (i) 1500 mg durvalumab or an antigen-binding fragment thereof is administered and (ii) 3000 mg oleclumab or an antigen-binding fragment thereof is administered every 2 weeks.

In some aspects, the tumor is a metastatic microsatellite-stable colorectal cancer tumor, and wherein (i) 1500 mg durvalumab or an antigen-binding fragment thereof is administered every 4 weeks; (ii) 3000 mg oleclumab or an antigen-binding fragment thereof is administered every 2 weeks for four doses and then every 4 weeks; (iii) the chemotherapy comprises (a) 400 mg/m² of folinic acid every 2 weeks (b) 85 mg/m² oxaliplatin every 2 weeks; and (c) 2400 mg/m² of 5-fluorouracil every 2 weeks; and (iv) 5 mg/kg of bevacizumab or an antigen-binding fragment thereof is administered every 2 weeks.

In some aspects, the tumor is a microsatellite-stable colorectal cancer tumor, and wherein (i) 1500 mg durvalumab or an antigen-binding fragment thereof is administered every 4 weeks; (ii) 3000 mg oleclumab or an antigen-binding fragment thereof is administered every 2 weeks for four or five doses and then every 4 weeks; and (iii) the chemotherapy comprises (a) 400 mg/m² of folinic acid every 2 weeks (b) 85 mg/m² oxaliplatin every 2 weeks; and (c) 400 mg/m² of 5-fluorouracil on day 1 and then 2400 mg/m² of 5-fluorouracil every 2 weeks.

In some aspects, the patient has an Eastern Cooperative Oncology Group (ECOG) performance status of 0 or 1.

### BRIEF DESCRIPTION OF THE DRAWINGS

FIG. 1 is a graph demonstrating the concentration of free sCD73 in cynomolgus monkeys administered a dose of 0 mg/kg (Group 1), 1 mg/kg (Group 2), 10 mg/kg (Group 3) or 107.8 mg/kg (Group 4) of oleclumab. (See Example 2.)

FIGS. 2A and 2B show that oleclumab administration did not enhance key hole limpet hemocyanin T-dependent Antibody Response (KLH TDAR) in a dose-dependent manner. FIG. 2A shows the mean anti-KLH IgM titers following KLH immunization in monkeys administered a dose of 0 mg/kg, 1 mg/kg, 10 mg/kg or 107.8 mg/kg of oleclumab and FIG. 2B shows the mean anti-KLH IgG titers following KLH immunization in monkeys administered a dose of 0 mg/kg, 1 mg/kg, 10 mg/kg or 107.8 mg/kg of oleclumab. (See Example 2.)

FIGS. 3A and 3B show that oleclumab administration did not increase ex vivo IFN-γ or IL-2 production following KLH stimulation of peripheral blood mononuclear cells. FIG. 3A shows the mean KLH specific IFN-γ production following KLH immunization in monkeys administered a dose of 0 mg/kg, 1 mg/kg, 10 mg/kg or 107.8 mg/kg of oleclumab and FIG. 3B shows the KLH specific IL-2 production following KLH immunization in monkeys administered a dose of 0 mg/kg, 1 mg/kg, 10 mg/kg or 107.8 mg/kg of oleclumab. (See Example 2.)

FIG. 4 shows that lymphocryptovirus (LCV) was detected by qPCR in most samples obtained from monkeys administered a dose of 0 mg/kg, 1 mg/kg, 10 mg/kg or 107.8 mg/kg of oleclumab. (See Example 2.)

FIG. 5 shows model assumptions for a pharmacokinetic/pharmacodynamic model for use in the prediction of therapeutic human doses of oleclumab. A two-compartment model with linear and target-mediated drug disposition clearance was used to adequately describe the MEDI9447 serum concentration profiles. This model was used to describe the monkey pharmacokinetic data and then resulting parameters were allometrically scaled to humans to predict doses using simulation. (See Example 2.)

FIGS. 6A and 6B show cynomolgus pharmacokinetic modeling and human dose prediction. Cynomolgus serum MEDI9447 concentration profiles (FIG. 6A) were described adequately all three different dose levels by a non-linear model shown schematically in (FIG. 5). Following allometric scaling of the pharmacokinetics parameters from this model, human serum concentration profiles were simulated (FIG. 6B). A serum exposure target of 52 μg/mL was determined from the tumor suppression data in syngeneic mice and simulations suggested that doses equal to or higher than 15 mg/kg given every two weeks (Q2W) would attain and maintain adequate exposure during the entire dosing period that are expected to result in efficacy. (See Example 2.)

FIGS. 7A and 7B. FIG. 7A shows the study flow diagram for the dose escalation phase for oleclumab monotherapy arm (pancreatic cancer and microsatellite stable colorectal cancer (MSS-CRC)). FIG. 7B shows the study flow diagram for the dose-expansion and dose-escalation portions of the study administering oleclumab (MEDI9447) and durvalumab as combination therapy to human patients with advanced solid tumors (pancreatic cancer and MSS-CRC). (See Example 3.)

FIG. 8 shows the dosing schema for the screening, treatment, and follow-up periods of the study administering oleclumab (MEDI9447) as a monotherapy and oleclumab (MEDI9447)/durvalumab combination therapy to human patients. (See Example 3.)

FIGS. 9A and 9B show the amount of free soluble CD73 observed following administration of oleclumab either as a monotherapy (FIG. 9A) or in combination therapy with Durvalumab (FIG. 9B). sCD73, soluble CD73; PD LLOQ, lower limit of quantification. (See Example 3.)

FIGS. 10A-C show oleclumab decreased CD73 on peripheral T cells and tumor CD73 surface expression as measured by mean fluorescence intensity (MFI) (FIG. 10A) and percent CD73⁺ CD4 and CD8 cells; (FIG. 10B) in peripheral blood across all doses after administration of oleclumab; and (FIG. 10C) in peripheral T cells. SSC; side scatter. (See Example 3.)

FIGS. 11A-C. FIG. 11A shows the change in the percentage of CD73 staining tumor cells at a 2+ or 3+ intensity by immunohistochemistry 20 days after oleclumab administration at 10, 20 or 40 mg/kg in either pancreatic or colorectal cancer (CRC) subjects. FIG. 11B shows CD73 staining on tumors pre and post treatment with oleclumab. FIG. 11C shows the change in CD73 staining tumor cells at a 2+ or 3+ staining intensity and the change in CD8 TILs 20 days after oleclumab administration at 10, 20 or 40 mg/kg relative to baseline. (See Example 3.)

FIG. 12 shows that Oleclumab inhibited CD73 enzymatic activity in tumor microenvironment. Staining shows a decrease in free adenosine. (See Example 3.)

FIG. 13 shows mean PK profile from the oleclumab monotherapy study. Pooled PK data (N=116) across indication and monotherapy as well as combination with durva. (See Example 3.)

FIGS. 14A and 14B show that oleclumab demonstrated evidence of PD effect. FIG. 14A shows the change in percentage of tumor. FIG. 14B shows the change in CD8 TILs. (See Example 3.)

FIG. 15 shows the dose expansion phase for oleclumab in combination with durvalumab. (See Example 4.)

FIG. 16 shows the clinical activity of oleclumab in combination with durvalumab in MSS-CRC. Ongoing treatment for >600 days. (See Example 4.)

FIG. 17 shows the clinical activity of oleclumab in combination with durvalumab in pancreatic cancer. (See Example 4.)

FIG. 18 shows the clinical activity of oleclumab in combination with durvalumab in EGFRm NSCLC. (See Example 4.)

FIG. 19 shows the study flow diagram for the dose escalation phase for oleclumab in combination with durvalumab and chemotherapy (gemcitabine+nab-paclitaxel for subjects with 1 L metastatic PDAC [Cohort A]; mFOLFOX for subjects with 2 L metastatic PDAC [Cohort B]). (See Example 5.)

FIG. 20 shows the treatment regimen for the dose escalation phase for oleclumab in combination with durvalumab and chemotherapy (gemcitabine+nab-paclitaxel for subjects with 1 L metastatic PDAC [Cohort A]; mFOLFOX for subjects with 2 L metastatic PDAC [Cohort B]). (See Example 5.)

FIG. 21 shows the study flow diagram for the dose expansion phase for oleclumab in combination with durvalumab and chemotherapy. (See Example 5.)

FIG. 22 shows the treatment regimen for the dose expansion phase for oleclumab in combination with durvalumab and chemotherapy (gemcitabine+nab-paclitaxel for subjects with 1 L metastatic PDAC [Cohort A]. (See Example 5.)

FIG. 23 shows the treatment regimen for the dose expansion phase for oleclumab in combination with durvalumab and chemotherapy mFOLFOX for subjects with 2 L metastatic PDAC [Cohort B]. (See Example 5.)

FIGS. 24A-D show treatment regimens for patients with first-line Stage IV non-small cell lung cancer (NSCLC). FIG. 24A shows the durvalumab monotherapy dosing schedule. FIG. 24B shows the durvalumab+oleclumab dosing schedule. FIG. 24C shows the durvalumab+chemotherapy dosing schedule. FIG. 24D shows the durvalumab+chemotherapy+oleclumab dosing schedule. (See Example 6.)

FIGS. 25A-B show treatment regimens for locally advanced, unresectable, Stage III non-small cell lung cancer (NSCLC). FIG. 25A shows the durvalumab monotherapy dosing schedule. D=durvalumab; a=subjects receive durvalumab 1500 mg intravenous (IV) every 4 weeks (Q4W) on Day 1 of each cycle. FIG. 25B shows the durvalumab+oleclumab dosing schedule. D=durvalumab; O=oleclumab; a=subjects receive 1500 mg intravenous (IV) every 4 weeks (Q4W) on Day 1 of each cycle, and oleclumab 3000 mg IV every 2 weeks (Q2W) (Day 1 and Day 15) for Cycle 1 and Cycle 2, then Q4W starting on Cycle 3, Day 1 (D1). On days when durvalumab and oleclumab are administered, oleclumab is administered first. (See Example 7.)

FIGS. 26A-C show treatment regimens for resectable, early-stage non-small cell lung cancer (NSCLC). FIG. 26A shows the treatment over the course of the study duration. FIG. 26B shows the durvalumab monotherapy dosing schedule: subjects receive 1500 mg durvalumab intravenously (IV) every 2 weeks (Q4W) on Week 1, Day 1. W=week; D=Day; Du=durvalumab. FIG. 26C shows the durvalumab+oleclumab dosing schedule: subjects receive 1500 mg durvalumab IV Q4W on Week 1, Day 1, and 3000 mg oleclumab IV every 2 weeks (Q2W) on Week 1, Day 1 and Week 3, Day 1. W=week; D=Day; Du=durvalumab; O=oleclumab. (See Example 8.)

FIG. 27 shows treatment regimens for metastatic microsatellite-stable colorectal cancer. DLT=dose-limiting toxicity; FOLFOX=folinic acid (leucovorin), 5-flurouracil, oxaliplatin. Subjects in Control 1 receive FOLFOX plus bevacizumab. Subjects in Arms 51 and E1 also receive durvalumab 1500 mg intravenous (IV) every 4 weeks (Q4W) on Day 1 of every other 14-day cycle, and oleclumab 3000 mg IV every 2 weeks (Q2W)×4 doses starting Cycle 1, Day 1, then Q4W starting on Cycle 5, Day 1. On days when durvalumab and oleclumab are administered, oleclumab is administered first. (See Example 9.)

FIG. 28 shows treatment regimens for high risk metastatic microsatellite-stable colorectal cancer. mFOLFOX6=folinic acid (leucovorin), 5-fluorouracil, oxaliplatin. Subjects in the Control Arm receive mFOLFOX6. Subjects in Arm E1-COC receive mFOLFOX6 plus durvalumab 1500 mg intravenous (IV) every 4 weeks (Q4W). Subjects in Arm E2 receive mFOLFOX6 plus durvalumab 1500 mg IV Q4W and oleclumab 300 mg IV every 2 weeks (Q2W) for four doses then Q4W starting Cycle 5 (Week 9, Day 1). On days when durvalumab and oleclumab are administered, oleclumab is administered first. (See Example 10.)

FIGS. 29A-I. FIG. 29A-H show individual tumor growth (CT26) post-treatment with a combination of anti-CD73, anti-PD-L1, 5FU, and OHP. FIG. 29I shows a Kaplan-Meier (survival curve) of CT26 tumor-bearing BALB/c mice post treatment with a combination of anti-CD73, anti-PD-L1, 5FU and OHP. (See Example 11.)

FIG. 30 shows an increase in IFNγ+CD8+, CD4+ and NKp46+ lymphocytes in the tumor microenvironment (TME) of CT26 tumor-bearing BALB/c mice post-treatment with a combination of anti-CD73, anti-PD-L1, 5FU, and OHP. (See Example 11.)

FIGS. 31A-I. FIG. 31A-H show the individual tumor growth (CT26) post-treatment with a combination of anti-CD73, anti-PD-L1, and docetaxel. FIG. 31I shows a Kaplan-Meier (survival curve) of CT26 tumor-bearing BALB/c mice post-treatment with a combination of anti-CD73, anti-PD-L1, and docetaxel. (See Example 11.)

FIGS. 32A-H show the individual tumor growth profiles of MCA205 tumor-bearing C57BL/6 mice post-treatment with a combination of anti-CD73, anti-PD-L1, 5FU, and OHP. (See Example 11.)

### DETAILED DESCRIPTION

This disclosure relates to a monoclonal antibody directed against CD73, such as oleclumab, or an antigen-binding fragment thereof, and the use of such antibody or antigen-binding fragment thereof in the treatment of tumors. The disclosure also relates to methods for the treatment of a tumor comprising administering to a patient in need thereof an anti-CD73 antibody, such as oleclumab, or antigen-binding fragment thereof in combination with a monoclonal antibody directed against PD-L1, such as durvalumab, or an antigen-binding fragment thereof. The disclosure also relates to methods for the treatment of a tumor comprising administering to a patient in need thereof an anti-CD73 antibody, such as oleclumab, or antigen-binding fragment thereof in combination with an anti-PD-L1 antibody such as durvalumab, in combination with chemotherapy.

As utilized in accordance with the present disclosure, the following terms, unless otherwise indicated, shall be understood to have the following meanings. Unless otherwise required by context, singular terms shall include pluralities and plural terms shall include the singular.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxy-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “antigen-binding fragment” refers to a portion of an intact antibody and/or refers to the antigenic determining variable domains of an intact antibody. It is known that the antigen-binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

The term “patient” as used herein includes human subjects.

A “disorder” is any condition that would benefit from treatment using the antibodies of the disclosure. “Disorder” and “condition” are used interchangeably herein and include chronic and acute disorders or diseases, including those pathological conditions that predispose a patient to the disorder in question.

The term “solid tumor” as used herein refers to an abnormal mass of tissue that normally does not contain cysts or liquid areas. Examples of solid tumors include squamous cell carcinoma of the head and neck, cervical cancer, colorectal cancer, non-small cell lung cancer, pancreatic cancer, prostate cancer, and urothelial bladder cancer.

The terms “treatment” or “treat” as used herein refer to both therapeutic treatment and prophylactic or preventative measures. Those in need of treatment include patients having a tumor as well as those prone to have a tumor or those in which a tumor is to be prevented. In particular embodiments, the antibodies disclosed herein can be used to treat tumors such as solid tumors. In particular embodiments, treatment of a tumor includes inhibiting tumor growth, promoting tumor reduction, or both.

The terms “pharmaceutical composition” or “therapeutic composition” as used herein refer to a compound or composition capable of inducing a desired therapeutic effect when properly administered to a patient. One embodiment of the disclosure provides a pharmaceutical composition comprising a pharmaceutically acceptable carrier and a therapeutically effective amount of at least one antibody of the disclosure.

The term “pharmaceutically acceptable carrier” or “physiologically acceptable carrier” as used herein refers to one or more formulation materials suitable for accomplishing or enhancing the delivery of one or more antibodies of the disclosure.

The terms “oleclumab” and “MEDI9447” as used herein refer to a human immunoglobulin G1 lambda (IgG1λ) mAb that selectively binds to and inhibits the ectonucleotidase activity of CD73, as disclosed in U.S. Pat. No. 9,938,356, which is incorporated by reference herein in its entirety. The triple mutation, L234F/L235E/P331S (according to European Union numbering convention), is encoded in the heavy chain constant region to significantly reduce IgG effector function. Oleclumab inhibits the catalysis of AMP to adenosine and organic phosphate by CD73. Extracellular adenosine mediates the immunosuppressive effects of both MDSCs and Tregs, among others.

In particular embodiments, oleclumab or an antigen binding fragment thereof comprises a heavy chain variable domain and a light chain variable domain. In particular embodiments, oleclumab comprises a light chain variable domain comprising the amino acid sequence of SEQ ID NO: 1 and a heavy chain variable domain comprising the amino acid sequence of SEQ ID NO: 2. In other embodiments, oleclumab or an antigen-binding fragment thereof comprises a heavy chain variable domain and a light chain variable domain, wherein the heavy chain variable domain comprises CDR1, CDR2, and CDR3 sequences of SEQ ID NOs: 3-5, and wherein the light chain variable domain comprises CDR1, CDR2, and CDR3 sequences of SEQ ID NOs: 6-8.

In particular embodiments, a tumor in a human patient is treated by administering oleclumab or an antigen-binding fragment thereof to the patient.

The monotherapy dose of oleclumab or an antigen-binding fragment thereof to be administered to the patient will vary depending, in part, upon the size (body weight, body surface, or organ size) and condition (the age and general health) of the patient.

In particular embodiments, the patient is administered one or more doses of oleclumab or an antigen-binding fragment thereof as a monotherapy or in a combination therapy, wherein the dose of oleclumab or an antigen-binding fragment thereof is 750 mg to 3000 mg. In some embodiments, the dose of the oleclumab or an antigen-binding fragment thereof is 750 mg. In some embodiments, the dose of the oleclumab or an antigen-binding fragment thereof is 1500 mg. In some embodiments, the dose of the oleclumab or an antigen-binding fragment thereof is 2250 mg. In some embodiments, the dose of the oleclumab or an antigen-binding fragment thereof is 3000 mg.

In particular embodiments, the patient is administered one or more doses of oleclumab or an antigen-binding fragment thereof as a monotherapy, wherein the dose is, 5 mg/kg, 10 mg/kg, 20 mg/kg, or 40 mg/kg. In some embodiments, the patient is administered one or more doses of oleclumab or an antigen-binding fragment thereof as a monotherapy wherein the dose is 40 mg/kg.

In particular embodiments, a patient presenting with a tumor is administered oleclumab or an antigen-binding fragment thereof only once or infrequently while still providing benefit to the patient. In further embodiments, the patient is administered additional follow-on doses. Follow-on doses can be administered at various time intervals depending on the patient's age, weight, clinical assessment, tumor burden, and/or other factors, including the judgment of the attending physician.

In particular embodiments, a bolus loading dose of oleclumab or an antigen-binding fragment thereof is administered to a patient presenting with a tumor. In particular embodiments, a patient will be administered a first dose of oleclumab or an antigen-binding fragment thereof followed by a second lower dose of oleclumab or an antigen-binding fragment thereof. The second lower dose can be repeated every 14 days to 28 days. In particular embodiments, the first dose of oleclumab is 40 mg/kg and the second lower dose of oleclumab is 20 mg/kg.

In particular embodiments, oleclumab or an antigen-binding fragment thereof is administered over a two-week treatment period, over a four-week treatment period, over a six-week treatment period, over an eight-week treatment period, over a twelve-week treatment period, over a twenty-four-week treatment period, or over a one-year or more treatment period. In particular embodiments, oleclumab or an antigen-binding fragment thereof is administered over a three-week treatment period, over a six-week treatment period, over a nine-week treatment period, over a twelve-week treatment period, over a twenty-four-week treatment period, or over a one-year or more treatment period. In particular embodiments, oleclumab or an antigen-binding fragment thereof is administered over a two-month treatment period, over a four-month treatment period, or over a six-month or more treatment period. In particular embodiments, oleclumab or an antigen-binding fragment thereof is administered over a one-year treatment period, over a two-year treatment period, over a three-year or more treatment period.

In particular embodiments, oleclumab or an antigen-binding fragment thereof is administered every week, every two weeks, every four weeks, every six weeks, every eight weeks, every 10 weeks, or every twelve weeks.

In particular embodiments, the administration of oleclumab or an antigen-binding fragment thereof is repeated every 7 to 28 days. In other embodiments, the administration of oleclumab or an antigen-binding fragment thereof is repeated every 14 days. In further embodiments, the administration of oleclumab or an antigen-binding fragment thereof is repeated every 28 days.

In particular embodiments, the administration of oleclumab or an antigen-binding fragment thereof is repeated every 7 to 28 days (e.g., every 7 days, every 14 days, every 21 days or every 28 days). In particular embodiments, oleclumab or an antigen-binding fragment thereof is administered every 14 days for at least two doses (e.g., for two, three, or four doses) and then every 28 days. In particular embodiments, oleclumab or an antigen-binding fragment thereof is administered every 21 days for at least two doses (e.g., for two, three, or four doses) and then every 28 days.

Also provided herein are methods for treating a solid tumor in a human patient, comprising administering 40 mg/kg of oleclumab or an antigen-binding fragment thereof to the patient. Also provided herein are methods for treating a solid tumor in a human patient, comprising administering 750-3000 mg (e.g., 750, 1500, 2250, or 3000 mg) of oleclumab or an antigen-binding fragment thereof to the patient.

In other embodiments, oleclumab or an antigen-binding fragment thereof can be administered in a combination therapy with durvalumab or an antigen-binding fragment thereof.

The term “durvalumab” as used herein refers to an antibody that selectively binds PD-L1 and blocks the binding of PD-L1 to the PD-1 and CD80 receptors, as disclosed in U.S. Pat. No. 9,493,565, which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (ADCC).

In particular embodiments, durvalumab or an antigen-binding fragment thereof comprises a heavy chain variable domain and a light chain variable domain. In particular embodiments, durvalumab comprises a light chain variable domain comprising the amino acid sequence of SEQ ID NO: 9 and a heavy chain variable domain comprising the amino acid sequence of SEQ ID NO: 10. In other embodiments, durvalumab or an antigen-binding fragment thereof comprises a heavy chain variable domain and a light chain variable domain, wherein the heavy chain variable domain comprises CDR1, CDR2, and CDR3 sequences of SEQ ID NOs: 11-13, and wherein the light chain variable domain comprises CDR1, CDR2, and CDR3 sequences of SEQ ID NOs: 14-16.

In particular embodiments, a patient presenting with a tumor is administered oleclumab or an antigen-binding fragment thereof in combination with durvalumab or an antigen-binding fragment thereof only once or infrequently while still providing benefit to the patient. In further embodiments, the patient is administered additional follow-on doses. Follow-on doses can be administered at various time intervals depending on the patient's age, weight, clinical assessment, tumor burden, and/or other factors, including the judgment of the attending physician.

In particular embodiments, oleclumab or an antigen-binding fragment thereof administered in a combination therapy with durvalumab or an antigen-binding fragment thereof is administered over a two-week treatment period, over a four-week treatment period, over a six-week treatment period, over an eight-week treatment period, over a twelve-week treatment period, over a twenty-four-week treatment period, or over a one-year or more treatment period. In particular embodiments, oleclumab or an antigen-binding fragment thereof is administered over a three-week treatment period, over a six-week treatment period, over a nine-week treatment period, over a twelve-week treatment period, over a twenty-four-week treatment period, or over a one-year or more treatment period. In particular embodiments, oleclumab or an antigen-binding fragment thereof administered is administered over a two-month treatment period, over a four-month treatment period, or over a six-month or more treatment period.

In particular embodiments, oleclumab or an antigen-binding fragment thereof administered in a combination therapy with durvalumab or an antigen-binding fragment thereof is administered every two weeks, every three weeks, every four weeks, every six weeks, every eight weeks, every 10 weeks, or every twelve weeks. In particular embodiments, oleclumab or an antigen-binding fragment thereof in a combination therapy with durvalumab or an antigen-binding fragment thereof is administered over a one-year treatment period, over a two-year treatment period, over a three-year or more treatment period.

In particular embodiments, the administration of oleclumab or an antigen-binding fragment thereof in a combination therapy with durvalumab or an antigen-binding fragment thereof is repeated every 14 to 28 days. In other embodiments, the administration of oleclumab or an antigen-binding fragment thereof is repeated every 14 days. In other embodiments, the administration of oleclumab or an antigen-binding fragment thereof is repeated every 21 days. In further embodiments, the administration of oleclumab or an antigen-binding fragment thereof is repeated every 28 days. In further embodiments, the administration of oleclumab or an antigen-binding fragment thereof is every 14 days for at least two doses (e.g., for two, three, or four doses) and then every 28 days In further embodiments, the administration of oleclumab or an antigen-binding fragment thereof is every 21 days for at least two doses (e.g., for two, three, or four doses) and then every 28 days.

In particular embodiments, durvalumab is administered about as frequently as oleclumab. In particular embodiments, the administration of durvalumab is repeated every 14 to 28 days. In other embodiments the administration of durvalumab is repeated every 14 days. In other embodiments the administration of durvalumab is repeated every 21 days. In further embodiments, the administration of durvalumab is repeated every 28 days. In further embodiments, the administration of durvalumab is repeated every 21 days for at least two doses (e.g., for two doses, three doses, or four doses), and then repeated every 28 days.

The combination therapy dose of oleclumab or an antigen-binding fragment thereof with durvalumab will vary depending, in part, upon the size (body weight, body surface, or organ size) and condition (the age and general health) of the patient. In particular embodiments, the patient is administered one or more doses of oleclumab or an antigen-binding fragment thereof as a combination therapy wherein the dose is 5 mg/kg, 10 mg/kg, 20 mg/kg, or 40 mg/kg. In particular embodiments, the patient is administered one or more doses of oleclumab or an antigen-binding fragment thereof as a combination therapy wherein the dose of oleclumab or antigen-binding fragment thereof is 750 mg, 1500 mg, 2250 mg, or 3000 mg.

The combination therapy dose of durvalumab with oleclumab will vary depending, in part, upon the size (body weight, body surface, or organ size) and condition (the age and general health) of the patient. In particular embodiments, the patient is administered one or more doses of durvalumab or an antigen-binding fragment thereof as a combination therapy wherein the dose is 3 mg/kg, 10 mg/kg or 20 mg/kg. In particular embodiments, the patient is administered one or more doses of durvalumab or an antigen-binding fragment thereof as a combination therapy wherein the dose of durvalumab or antigen-binding fragment thereof is 1500 mg.

In particular embodiments, a bolus loading dose of oleclumab or an antigen-binding fragment and/or durvalumab or an antigen-binding fragment thereof is administered to a patient presenting with a tumor. In particular embodiments, a patient will be administered a first dose of oleclumab or an antigen-binding fragment and/or durvalumab or an antigen-binding fragment followed by a second lower dose of oleclumab or an antigen-binding fragment and/or durvalumab or an antigen-binding fragment thereof.

In particular embodiments, the patient is administered 2 mg/kg oleclumab or an antigen-binding fragment thereof every two weeks and 10 mg/kg of durvalumab or an antigen-binding fragment thereof every 2 weeks. In particular embodiments, the patient is administered 5 mg/kg oleclumab or an antigen-binding fragment thereof every two weeks and 10 mg/kg of durvalumab or an antigen-binding fragment thereof every 2 weeks. In particular embodiments, the patient is administered 10 mg/kg oleclumab or an antigen-binding fragment thereof every two weeks and 10 mg/kg of durvalumab or an antigen-binding fragment thereof every 2 weeks. In particular embodiments, the patient is administered 20 mg/kg oleclumab or an antigen-binding fragment thereof every two weeks and 10 mg/kg of durvalumab or an antigen-binding fragment thereof every 2 weeks. In particular embodiments, the patient is administered 40 mg/kg oleclumab or an antigen-binding fragment thereof every two weeks and 10 mg/kg of durvalumab or an antigen-binding fragment thereof every 2 weeks.

In particular embodiments, the patient is administered 2 mg/kg oleclumab or an antigen-binding fragment thereof every four weeks and 10 mg/kg of durvalumab or an antigen-binding fragment thereof every 2 weeks. In particular embodiments, the patient is administered 5 mg/kg oleclumab or an antigen-binding fragment thereof every four weeks and 10 mg/kg of durvalumab or an antigen-binding fragment thereof every 2 weeks. In particular embodiments, the patient is administered 10 mg/kg oleclumab or an antigen-binding fragment thereof every four weeks and 10 mg/kg of durvalumab or an antigen-binding fragment thereof every 2 weeks. In particular embodiments, the patient is administered 20 mg/kg oleclumab or an antigen-binding fragment thereof every four weeks and 10 mg/kg of durvalumab or an antigen-binding fragment thereof every 2 weeks. In particular embodiments, the patient is administered 40 mg/kg oleclumab or an antigen-binding fragment thereof every four weeks and 10 mg/kg of durvalumab or an antigen-binding fragment thereof every 2 weeks.

In particular embodiments, the patient is administered 2 mg/kg oleclumab or an antigen-binding fragment thereof every four weeks and 20 mg/kg of durvalumab or an antigen-binding fragment thereof every four weeks. In particular embodiments, the patient is administered 5 mg/kg oleclumab or an antigen-binding fragment thereof every four weeks and 20 mg/kg of durvalumab or an antigen-binding fragment thereof every four weeks. In particular embodiments, the patient is administered 10 mg/kg oleclumab or an antigen-binding fragment thereof every four weeks and 20 mg/kg of durvalumab or an antigen-binding fragment thereof every four weeks. In particular embodiments, the patient is administered 20 mg/kg oleclumab or an antigen-binding fragment thereof every four weeks and 20 mg/kg of durvalumab or an antigen-binding fragment thereof every four weeks. In particular embodiments, the patient is administered 40 mg/kg oleclumab or an antigen-binding fragment thereof every four weeks and 20 mg/kg of durvalumab or an antigen-binding fragment thereof every four weeks.

In particular embodiments, the patient is administered 1500 mg or 3000 mg of oleclumab or antigen binding fragment thereof every 2 weeks for two 28-day cycles doses and then every 4 weeks and 1500 mg durvalumab or an antigen-binding fragment thereof every 4 weeks.

In particular embodiments, the patient is administered 3000 mg of oleclumab or antigen binding fragment thereof every 2 weeks and 1500 mg durvalumab or an antigen-binding fragment thereof.

In particular embodiments, the patient is administered 3000 mg of oleclumab or antigen binding fragment thereof every 2 weeks for two months and then every 4 weeks and 1500 mg durvalumab or an antigen-binding fragment thereof every 4 weeks.

In other embodiments, oleclumab or an antigen-binding fragment thereof can be administered in a combination therapy with chemotherapy.

In particular embodiments, a patient presenting with a tumor is administered oleclumab or an antigen-binding fragment thereof in combination with chemotherapy only once or infrequently while still providing benefit to the patient. In further embodiments, the patient is administered additional follow-on doses. Follow-on doses can be administered at various time intervals depending on the patient's age, weight, clinical assessment, tumor burden, and/or other factors, including the judgment of the attending physician.

In particular embodiments, oleclumab or an antigen-binding fragment thereof in combination with chemotherapy is administered over a two-week treatment period, over a three-week period, over a four-week treatment period, over a six-week treatment period, over an eight-week treatment period, over a twelve-week treatment period, over a twenty-four-week treatment period, or over a one-year or more treatment period. In particular embodiments, oleclumab or an antigen-binding fragment thereof is administered over a three-week treatment period, over a six-week treatment period, over a nine-week treatment period, over a twelve-week treatment period, over a twenty-four-week treatment period, or over a one-year or more treatment period. In particular embodiments, oleclumab or an antigen-binding fragment thereof is administered over a two-month treatment period, over a four-month treatment period, or over a six-month or more treatment period. In particular embodiments, oleclumab or an antigen-binding fragment thereof in a combination therapy with chemotherapy is administered over a one-year treatment period, over a two-year treatment period, over a three-year or more treatment period.

In particular embodiments, oleclumab or an antigen-binding fragment thereof in combination with chemotherapy is administered every two weeks, every three weeks. every four weeks, every six weeks, every eight weeks, every 10 weeks, or every twelve weeks.

In particular embodiments, the administration of oleclumab or an antigen-binding fragment thereof in combination with chemotherapy is repeated every 14 to 28 days. In other embodiments, the administration of oleclumab or an antigen-binding fragment thereof in is repeated every 14 days. In other embodiments, the administration of oleclumab or an antigen-binding fragment thereof in is repeated every 21 days. In further embodiments, the administration of oleclumab or an antigen-binding fragment thereof in is repeated every 28 days. In further embodiments, the administration of oleclumab or an antigen-binding fragment thereof is every 14 days for at least two doses (e.g., for two, three, or four doses) and then every 28 days In further embodiments, the administration of oleclumab or an antigen-binding fragment thereof is every 21 days for at least two doses (e.g., for two, three, or four doses) and then every 28 days.

The combination therapy dose of oleclumab or an antigen-binding fragment thereof with chemotherapy will vary depending, in part, upon the size (body weight, body surface, or organ size) and condition (the age and general health) of the patient. In particular embodiments, the patient is administered one or more doses of oleclumab or an antigen-binding fragment thereof in combination with chemotherapy wherein the dose of oleclumab or an antigen-binding fragment thereof in is 750 mg, 1500 mg or 3000 mg. In particular embodiments, the patient is administered one or more doses of oleclumab or an antigen-binding fragment thereof in combination with chemotherapy wherein the dose of oleclumab or an antigen-binding fragment thereof in is 2250 mg.

In particular embodiments, the chemotherapy comprises at least one of cisplatin, pemetrexed. nab-paclitaxel, carboplatin, gemcitabine, cisplatin, oxaliplatin, leucovorin, 5-fluorouracil, and docetaxel. In particular embodiments, the chemotherapy comprises a combination of at least two of cisplatin, pemetrexed. nab-paclitaxel, carboplatin, gemcitabine, cisplatin, oxaliplatin, leucovorin, 5-fluorouracil, and docetaxel. In particular embodiments, the chemotherapy comprises oxaliplatin, leucovorin, and 5-fluorouracil. In particular embodiments, the chemotherapy comprises nab-paclitaxel and carboplatin. In particular embodiments, the chemotherapy comprises gemcitabine and cisplatin. In particular embodiments, the chemotherapy comprises gemcitabine and carboplatin. In particular embodiments, the chemotherapy comprises pemetrexed and carboplatin. In particular embodiments, the chemotherapy comprises pemetrexed and cisplatin.

In particular embodiments, the chemotherapy comprises at least one of gemcitabine, nab-paclitaxel, oxaliplatin, leucovorin and 5-fluorouracil.

The combination therapy dose of chemotherapy with oleclumab will vary depending, in part, upon the size (body weight, body surface, or organ size) and condition (the age and general health) of the patient. Oleclumab can be used in combination with chemotherapy utilizing any chemotherapy regimen known in the art. In particular embodiments, the patient is administered one or more doses of gemcitabine at a dose of 1000 mg/m² and nab-paclitaxel at a dose of 125 mg/m². In particular embodiments, the patient is administered one or more doses of oxaliplatin at a dose of 85 mg/m², leucovorin at a dose of 400 mg/m² and 5-fluorouracil at a dose of 2400 mg/m².

In particular embodiments, the patient is administered one or more doses of 85 mg/m² oxaliplatin. In particular embodiments, the patient is administered one or more doses of 400 mg/m² leucovorin. In particular embodiments, the patient is administered one or more doses of 2400 mg/m² 5-fluorouracil (5-FU). In particular embodiments, the patient is administered one or more doses of 85 mg/m² oxaliplatin, 400 mg/m² leucovorin and 2400 mg/m² 5-fluorouracil.

In particular embodiments, the patient is administered one or more doses of 100 mg/m² nab-paclitaxel. In particular embodiments, the patient is administered one or more doses of 1000 or 1250 mg/m² gemcitabine. In particular embodiments, the patient is administered one or more doses of 500 mg/m² pemetrexed. In particular embodiments, the patient is administered one or more doses of AUC 5 or 6 carboplatin. In particular embodiments, the patient is administered one or more doses of 75 mg/m² cisplatin.

In particular embodiments, chemotherapy is administered about as frequently as oleclumab. In particular embodiments, the administration of chemotherapy is repeated every 7 to 28 days. In other embodiments the administration of chemotherapy is repeated every 7 days. In other embodiments the administration of chemotherapy is repeated every 14 days. In other embodiments the administration of chemotherapy is repeated every 21 days. In further embodiments, the administration of chemotherapy is repeated every 28 days. In other embodiments the administration of chemotherapy is repeated on days 1 and 8 of a 21-day cycle

In other embodiments, oleclumab or an antigen-binding fragment thereof can be administered in a combination therapy with chemotherapy and durvalumab or an antigen-binding fragment thereof.

In particular embodiments, the combination therapy including oleclumab, durvalumab and chemotherapy, durvalumab is administered about as frequently as oleclumab. In particular embodiments, oleclumab is administered about twice as frequently as durvalumab. In particular embodiments, the administration of durvalumab is repeated every 14 to 28 days. In other embodiments the administration of durvalumab is repeated every 14 days. In further embodiments, the administration of durvalumab is repeated every 21 days. In further embodiments, the administration of durvalumab is repeated every 28 days. In further embodiments, the administration of durvalumab is repeated every 21 days for at least two doses (e.g., for two doses, three doses, or four doses), and then repeated every 28 days.

The combination therapy dose of durvalumab or an antigen-binding fragment thereof with oleclumab or an antigen-binding fragment thereof and chemotherapy will vary depending, in part, upon the size (body weight, body surface, or organ size) and condition (the age and general health) of the patient. In particular embodiments, the patient is administered one or more doses of durvalumab or an antigen-binding fragment thereof as a combination therapy wherein the dose or durvalumab or an antigen-binding fragment thereof is 1500 mg.

In particular embodiments, the patient is administered 750 mg of oleclumab or an antigen-binding fragment thereof every 2 weeks for four doses and then every 4 weeks, 1500 mg of durvalumab or an antigen-binding fragment thereof every 4 weeks, and 1000 mg/m² gemcitabine and 125 mg/m² nab-paclitaxel on days 1, 8 and 15 then every 4 weeks. In particular embodiments, the patient is administered 1500 mg of oleclumab or an antigen-binding fragment thereof every 2 weeks for four doses and then every 4 weeks, 1500 mg of durvalumab or an antigen-binding fragment thereof every 4 weeks, and 1000 mg/m² gemcitabine and 125 mg/m² nab-paclitaxel on days 1, 8 and 15 then every 4 weeks. In particular embodiments, the patient is administered 3000 mg of oleclumab or an antigen-binding fragment thereof every 2 weeks for four doses and then every 4 weeks, 1500 mg of durvalumab or an antigen-binding fragment thereof every 4 weeks, and 1000 mg/m² gemcitabine and 125 mg/m² nab-paclitaxel on days 1, 8 and 15 then every 4 weeks.

In particular embodiments, the patient is administered 750 mg of oleclumab or an antigen-binding fragment thereof every 2 weeks for four doses and then every 4 weeks, 1500 mg of durvalumab or an antigen-binding fragment thereof every 4 weeks, and 85 mg/m² oxaliplatin, 400 mg/m² leucovorin and 2400 mg/m² 5-fluorouracil on days 1 and 15 and then every 4 weeks. In particular embodiments, the patient is administered 1500 mg of oleclumab or an antigen-binding fragment thereof every 2 weeks for four doses and then every 4 weeks, 1500 mg of durvalumab or an antigen-binding fragment thereof every 4 weeks, and 85 mg/m² oxaliplatin, 400 mg/m² leucovorin and 2400 mg/m² 5-fluorouracil on days 1 and 15 and then every 4 weeks. In particular embodiments, the patient is administered 3000 mg of oleclumab or an antigen-binding fragment thereof every 2 weeks for four doses and then every 4 weeks, 1500 mg of durvalumab or an antigen-binding fragment thereof every 4 weeks, and 85 mg/m² oxaliplatin, 400 mg/m² leucovorin and 2400 mg/m² 5-fluorouracil on days 1 and 15 and then every 4 weeks.

In particular embodiments, the patient is administered 1500 mg or 3000 mg of oleclumab or antigen binding fragment thereof every 2 weeks for four doses and then every 4 weeks and chemotherapy comprising 1000 mg/m² gemcitabine and 125 mg/m² nab-paclitaxel. The chemotherapy can be administered on days 1, 8, and 15 of four 28-day cycles and then every 4 weeks. In addition, 1500 mg durvalumab or an antigen-binding fragment thereof can be administered every 4 weeks.

In particular embodiments, the patient is administered 1500 mg or 3000 mg of oleclumab or antigen binding fragment thereof every 2 weeks for four doses and then every 4 weeks and chemotherapy comprising 85 mg/m² oxaliplatin, 400 mg/m² leucovorin, and 400 mg/m² 5-FU followed by 2400 mg/m² 5-FU. The chemotherapy can be administered on days 1 and 15 of four 28-day cycles and then every 4 weeks. In addition, 1500 mg durvalumab or an antigen-binding fragment thereof can be administered every 4 weeks.

In particular embodiments, the patient is administered (i) oleclumab or antigen binding fragment thereof is administered (a) at 1500 mg every 3 weeks for four 21-day cycles and then every 4 weeks; or (b) at 2250 mg every 3 weeks for four 21-day cycles and then at 3000 mg every 4 weeks; (ii) durvalumab or antigen binding fragment thereof at 1500 mg every 3 weeks for four 21-day cycles and then every 4 weeks; and (iii) chemotherapy comprising: (a) 100 mg/m² nab-paclitaxel on days 1, 8, and 15 of a 21-day cycle for 4 cycles and 5 or 6 AUC carboplatin on day 1 of the 21-day cycle for 4 cycles; (b) 1000 mg/m² or 1250 mg/m² gemcitabine on days 1 and 8 of a 21-day cycle for 4 cycles and 75 mg/m² cisplatin on day 1 of the 21-day cycle for 4 cycles; (c) 1000 mg/m² or 1250 mg/m² gemcitabine on days 1 and 8 of a 21-day cycle for 4 cycles and 5 or 6 AUC carboplatin on day 1 of the 21-day cycle for 4 cycles; (d) 500 mg/m² pemetrexed on day 1 of 21-day cycle for 4 cycles and 5 or 6 AUC carboplatin on day 1 of the 21-day cycle for 4 cycles; or (e) 500 mg/m² pemetrexed on day 1 of 21-day cycle for 4 cycles and 75 mg/m² cisplatin on day 1 of the 21-day cycle for 4 cycles. Pemetrexed can be further administered as a maintenance therapy, e.g., at 500 mg/m² every 4 weeks.

In particular embodiments, the patient is administered (i) 1500 mg durvalumab or an antigen-binding fragment thereof every 4 weeks; (ii) 3000 mg oleclumab or an antigen-binding fragment thereof every 2 weeks for five doses and then every 4 weeks; and (iii) chemotherapy comprising (a) 400 mg/m² of folinic acid every 2 weeks (b) 85 mg/m² oxaliplatin every 2 weeks; and (c) 400 mg/m² of 5-fluorouracil on day 1 and then 2400 mg/m2 of 5-fluorouracil every 2 weeks.

In other embodiments, bevacizumab or an antigen-binding fragment thereof can be administered in a combination therapy with oleclumab or an antigen-binding fragment thereof and chemotherapy (e.g., with chemotherapy comprising oxaliplatin, leucovorin and 5-fluorouracil).

The term “bevacizumab” as used herein refers to an antibody that selectively binds vascular endothelial growth factor (VEGF), as disclosed in U.S. Pat. Nos. 6,884,879 and 7,169,901, which are incorporated by reference herein in its entirety.

In particular embodiments, bevacizumab or an antigen-binding fragment thereof comprises a heavy chain variable domain and a light chain variable domain. In particular embodiments, bevacizumab comprises a light chain variable domain comprising the amino acid sequence of SEQ ID NO: 17 and a heavy chain variable domain comprising the amino acid sequence of SEQ ID NO: 18. In other embodiments, durvalumab or an antigen-binding fragment thereof comprises a heavy chain variable domain and a light chain variable domain, wherein the heavy chain variable domain comprises CDR1, CDR2, and CDR3 sequences of SEQ ID NOs: 19-21, and wherein the light chain variable domain comprises CDR1, CDR2, and CDR3 sequences of SEQ ID NOs: 22-24.

In particular embodiments, the patient is administered one or more doses of 5 mg/kg bevacizumab or an antigen-binding fragment thereof. The bevacizumab or an antigen-binding fragment thereof can be administered, e.g., every 2 weeks. The bevacizumab or an antigen-binding fragment thereof can be administered, e.g., intravenously.

In particular embodiments, the patient is administered (i) 1500 mg durvalumab or an antigen-binding fragment thereof every 4 weeks; (ii) 3000 mg oleclumab or an antigen-binding fragment thereof every 2 weeks for four doses and then every 4 weeks; (iii) chemotherapy comprising (a) 400 mg/m² of folinic acid every 2 weeks (b) 85 mg/m² oxaliplatin every 2 weeks; and (c) 2400 mg/m² of 5-fluorouracil every 2 weeks; and (iv) 5 mg/kg of bevacizumab or an antigen-binding fragment thereof is administered every 2 weeks.

In particular embodiments, the patient receiving treatment has a solid tumor that is a is a breast cancer, ovarian cancer, head and neck cancer, prostate cancer, bladder cancer, colorectal cancer, non-small cell lung cancer (NSCLC), glioblastoma, renal cell cancer, or pancreatic cancer.

In particular embodiments, the patient receiving treatment has a solid tumor such as colorectal cancer, non-small cell lung cancer, or pancreatic cancer. In particular embodiments, the patient has pancreatic ductal adenocarcinoma. In particular embodiments, the patient has metastatic pancreatic ductal adenocarcinoma that has not been previously treated. In particular embodiments, the patient has metastatic pancreatic ductal adenocarcinoma that was previously treated with gemcitabine-based therapy.

In particular embodiments, the patient has a resectable NSCLC tumor, an early-stage NSCLC tumor, or stage IV NSCLC tumor.

In particular embodiments, the patent has metastatic colorectal cancer.

In particular embodiments, the patient has a tumor with high-PD-L1 expression or with low-PD-L1 expression. The tumor with high-PD-L1 expression or low-PD-L1 expression can be a NSCLC.

In particular embodiments, the patient has a tumor that lacks an activating epidermal growth factor receptor (EGFR) mutation and/or an anaplastic lymphoma kinase (ALK) fusion. The tumor can be a NSCLC tumor.

In particular embodiments, the patient has a tumor has not received prior treatment in the recurrent and/or metastatic setting.

The antibodies of the disclosure can be selected for parenteral administration. For example, the antibodies of the disclosure can be administered by intravenous infusion or by subcutaneous injection. In particular embodiments, the administration is by intravenous infusion.

Response Evaluation Criteria In Solid Tumors (RECIST) refers to a set of published rules that define when cancer patients improve, stay the same or worsen during treatments. The types of response a patient can have are a complete response (CR), a partial response (PR), progressive disease (PD), and stable disease (SD).

The methods provided herein can be used for disease control (DC) of a tumor. Disease control can be a complete response (CR), partial response (PR), or stable disease (SD).

A “complete response” (CR) refers to the disappearance of all lesions, whether measurable or not, and no new lesions. Confirmation of a complete response can be obtained using a repeat, consecutive assessment no less than four weeks from the date of first documentation. New, non-measurable lesions preclude CR.

A “partial response” (PR) refers to a decrease in tumor burden of ≥50% relative to baseline. Confirmation can be obtained using a consecutive repeat assessment at least 4 weeks from the date of first documentation.

“Progressive disease” (PD) refers to an increase in tumor burden of ≥25% relative to the minimum recorded (nadir). Confirmation can be obtained by a consecutive repeat assessment at least 4 weeks from the date of first documentation. New, non-measurable lesions do not define PD.

“Stable disease” (SD) refers to not meeting the criteria for CR, PR, or PD.

Without limiting the disclosure, a number of embodiments of the disclosure are described below for purpose of illustration.

In one aspect (A1) of the methods provided herein, a method of treating a tumor in a human patient comprises administering oleclumab or antigen-binding fragment thereof to the patient.

In one aspect of A1 (A2), the oleclumab or antigen-binding fragment thereof is administered at a dose of 2 mg/kg, 5 mg/kg, 10 mg/kg, 20 mg/kg, or 40 mg/kg.

In another aspect of A1 (A3), the oleclumab or antigen-binding fragment thereof is administered at a dose of 40 mg/kg.

In one aspect of any one of A1-A3 (A4), the oleclumab or antigen-binding fragment thereof is administered every 14 to 28 days.

In one aspect of A4 (A5), the oleclumab or antigen-binding fragment thereof is administered every 14 days.

In another aspect of A4 (A6), the oleclumab or antigen-binding fragment thereof is administered every 28 days.

In one aspect of any one of A1-A6 (A7), the administration of oleclumab or antigen-binding fragment thereof results in a partial response.

In another aspect of any one of A1-A6 (A8), the administration of oleclumab or antigen-binding fragment thereof results in a complete response.

In another aspect of any one of A1-A6 (A9), the tumor is a solid tumor.

In one aspect of A9 (A10), the solid tumor is colorectal cancer, non-small cell lung cancer, or pancreatic cancer.

In one aspect of any one of A1-A10 (A11), the patient has an Eastern Cooperative Oncology Group (ECOG) performance status of 0 or 1.

In one aspect (A12) of the methods provided herein, a method of treating a solid tumor in a human patient comprises administering 40 mg/kg of oleclumab or antigen-binding fragment thereof to the patient.

In one aspect (A13) of the methods provided herein, a method of treating a tumor in a human patient comprises administering oleclumab or antigen-binding fragment thereof and durvalumab or antigen-binding fragment thereof to the patient.

In one aspect of A13 (A14), the oleclumab or antigen-binding fragment thereof is administered at a dose of 2 mg/kg, 5 mg/kg, 10 mg/kg, 20 mg/kg, or 40 mg/kg.

In one aspect of A13 or A14 (A15), the durvalumab or antigen-binding fragment thereof is administered at a dose of 3 mg/kg, 10 mg/kg or 20 mg/kg.

In one aspect of any one of A13-A15 (A16), the oleclumab or antigen-binding fragment thereof is administered every 14 days to 28 days.

In one aspect of any one of A13-A16 (A17), the durvalumab or antigen-binding fragment thereof is administered every 14 days to 28 days.

In one aspect of any one of A13-A17 (A18), the tumor is a solid tumor.

In one aspect of A18 (A19), the solid tumor is colorectal cancer, non-small cell lung cancer, or pancreatic cancer.

In one aspect of any one of A13-A19 (A20), the patient has an Eastern Cooperative Oncology Group (ECOG) performance status of 0 or 1.

In one aspect (A21) of the methods provided herein, a method of treating a tumor in a human patient comprises administering oleclumab or antigen-binding fragment thereof and chemotherapy to the patient.

In one aspect of A21 (A22), the oleclumab or antigen-binding fragment thereof is administered at a dose of 750 mg, 1500 mg or 3000 mg.

In one aspect of A21 or A22 (A23), the oleclumab or antigen-binding fragment thereof is administered every 14 days to 28 days.

In one aspect of any one of A21-A23 (A24), the method further comprises administering durvalumab or antigen-binding fragment thereof.

In one aspect of A24 (A25), the durvalumab or antigen-binding fragment thereof is administered at a dose of 1500 mg.

In one aspect of A24 or A25 (A26), the durvalumab or antigen-binding fragment thereof is administered every 28 days.

In one aspect of any one of A21-A26 (A27), the chemotherapy comprises at least one of gemcitabine, nab-paclitaxel, oxaliplatin, leucovorin and 5-fluorouracil.

In one aspect of A27 (A28), the chemotherapy comprises 1000 mg/m² gemcitabine and 125 mg/m² nab-paclitaxel.

In another aspect of A27 (A29), the chemotherapy comprises 85 mg/m² oxaliplatin, 400 mg/m² leucovorin and 2400 mg/m² 5-fluorouracil.

In one aspect of any one of A21-A29 (A30), the chemotherapy is administered every 7 days to 28 days.

In one aspect of any one of A29-A30 (A31), 5-fluorouracil is administered by continuous intravenous infusion for 46 to 48 hours.

In one aspect of A28 (A32), the patient has metastatic pancreatic ductal adenocarcinoma that has not been previously treated.

In one aspect of A29 (A33), the patient has metastatic pancreatic ductal adenocarcinoma that was previously treated with gemcitabine-based therapy.

### EXAMPLES

The Examples that follow are illustrative of specific embodiments of the disclosure, and various uses thereof. They are set forth for explanatory purposes only, and should not be construed as limiting the scope of the invention in any way.

### Example 1: Evaluation of Toxicity of Oleclumab in Mouse and Monkey Species

Mouse and cynomolgus monkey were selected as pharmacologically relevant species for evaluation of the toxicity of oleclumab. This assessment was based on a composite of factors: (i) moderate-to-high protein sequence identity between mouse and cynomolgus monkey CD73 and human CD73 (86% and 98%, respectively); (ii) similar binding affinity of oleclumab for mouse, cynomolgus monkey, and human CD73; and (iii) similar potency of oleclumab against mouse, monkey, and human recombinant CD73 enzyme activity in cell-based in vitro assays. Additionally, in cynomolgus monkeys, single IV doses of ≥1 mg/kg oleclumab (lowest dose tested) suppressed soluble CD73 in the serum, with a dose-related duration of suppression. Systemic and local toxicities of oleclumab were evaluated in Good Laboratory Practice (GLP) toxicity studies in CD-1 mice (5-week, repeat intravenous [IV] bolus dose, once every 4 days, total 9 doses) at 0 mg/kg, 100 mg/kg or 200 mg/kg and cynomolgus monkeys (5-week, repeat IV 30-minute infusion dose, once weekly, total 5 doses) at 0 mg/kg, 30.5 mg/kg, 103.7 or 300.7 mg/kg. No oleclumab-related adverse effects were noted in CD-1 mice at doses up to 200 mg/kg or in cynomolgus monkeys at doses up to 300.7 mg/kg. There were also no oleclumab-related effects on safety pharmacology endpoints (electrocardiograms [ECGs], blood pressure, and behavioral examinations), which were evaluated as part of the 5-week cynomolgus monkey study. Therefore, the no-observed-adverse-effect level (NOAEL) was considered to be 200 mg/kg/dose (the highest dose tested; maximum observed concentration [Cₘₐₓ], 6,200 μg/mL; area under the concentration-time curve [AUC]₀₋₉₆ₕᵣ, 229,000 μg·hr/mL) in CD-1 mice and 300.7 mg/kg/dose (the highest dose tested; Cₘₐₓ, 11,000 μg/mL; AUC_{0-168.5hr}, 834,000 μg·hr/mL) in cynomolgus monkeys. In the GLP human tissue cross reactivity evaluation, staining with oleclumab was observed in multiple cell types throughout the human tissue panel examined.

The ability of oleclumab (alone or in combination with durvalumab) to induce cytokine release was evaluated in human in vitro assays using blood or peripheral blood mononuclear cell from healthy donors. Oleclumab alone or in combination with durvalumab, presented in solution or immobilized on plastic wells by dry-coating, did not induce cytokine release.

### Example 2: Single Dose Pharmacokinetic/Pharmacodynamic Cynomolgus Monkey Study

Five male cynomolgus monkeys were administered a single IV bolus dose of oleclumab at a dose of 0 mg/kg, 1 mg/kg, 10 mg/kg or 107.8 mg/kg. Serum was collected prior to the first dose at 0.08, 0.5, 2, 8 and 24 hours and 2, 3, 5, 7, 10, 14, 21, 28 and 35 days post dosing.

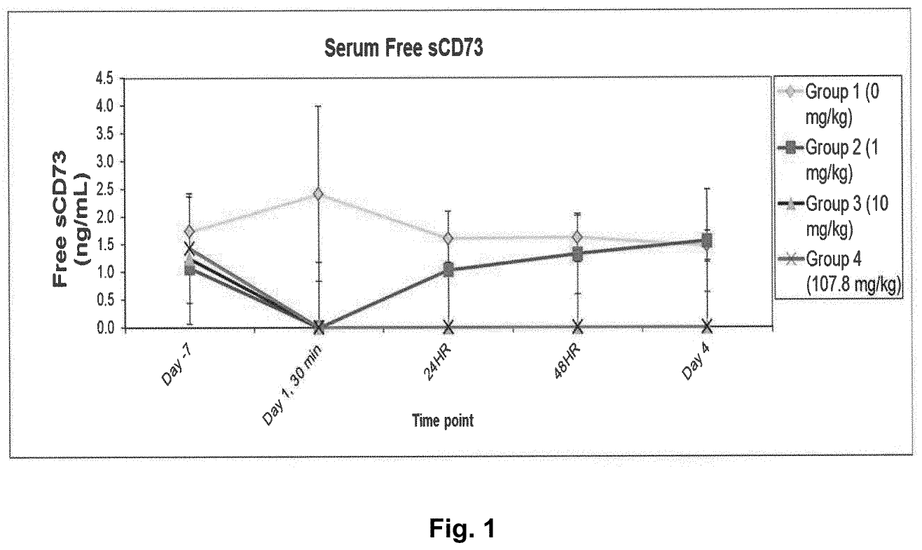

To detect free sCD73, plates were coated with 0.5 ug/mL anti-CD-73 antibody overnight and then 50 uL monkey serum was added to the plates. The plates were incubated for 15 min±1 min, washed and anti-CD73-HRP was added. Free sCD73 was suppressed in all dose groups, however suppression of free sCD73 was dose dependent (FIG. 1).

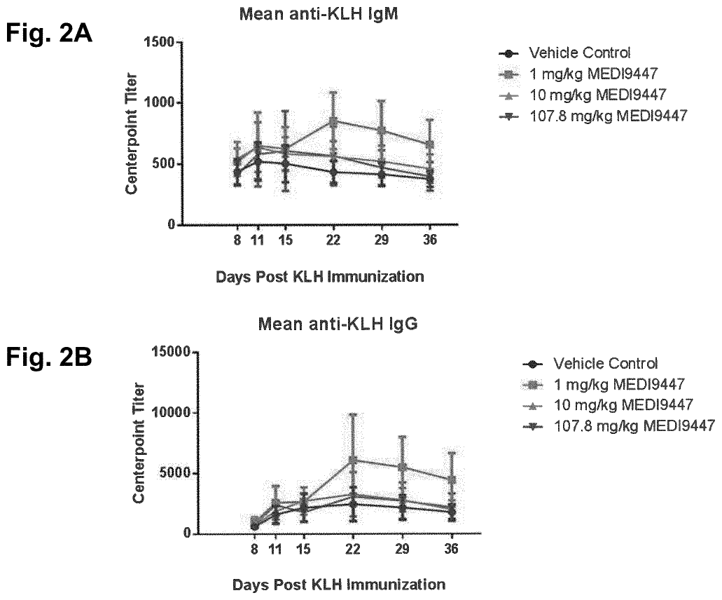

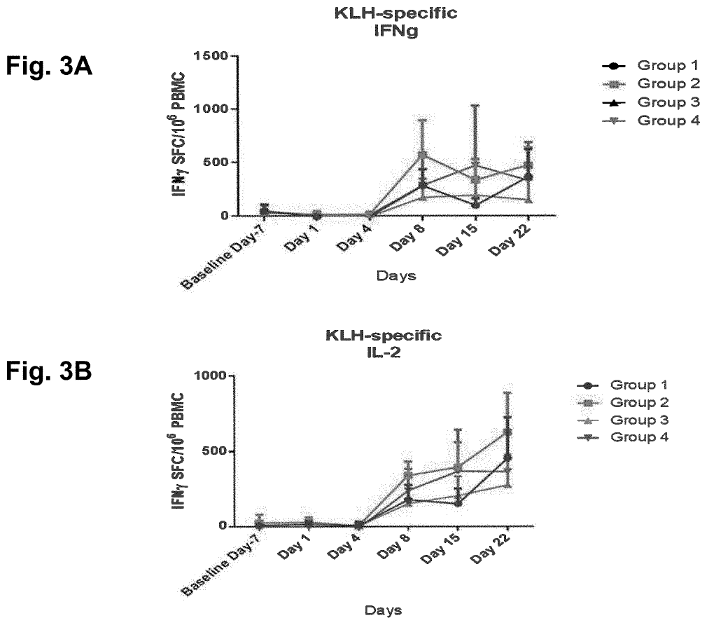

To determine immune modulation, the exogenous antigen (KLH) response was measured. The exogenous antigen (KLH) response was determined by first immunizing the monkeys on day 1 with adjuvant free KLH. KLH T cell-dependent antibody response (TDAR) was conducted on days 7, 8, 11, 15, 22, 29, and 36. Ex vivo KLH stimulation of PBMC IFN-γ and IL-2 ELISPOT was determined on days 1, 4, 8, 7, 15, and 22. Oleclumab did not enhance responses to exogenous antigen (KLH). Anti-KLH IgM and IgG antibody responses were detectable but did not show dose-dependent increase with oleclumab treatment (FIGS. 2A and 2B). T cell responses to ex vivo KLH stimulation were detectable but did not vary with oleclumab treatment (FIGS. 3A and 3B).

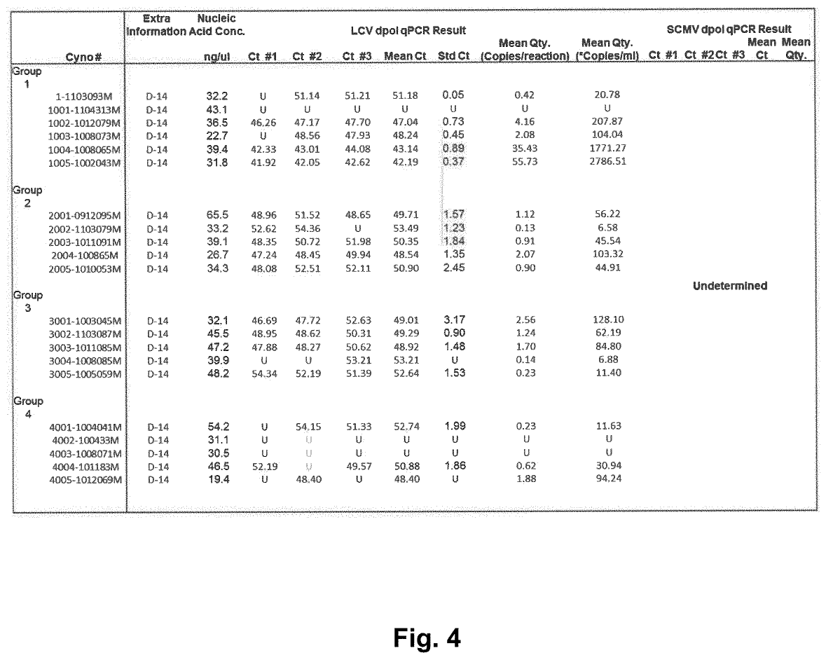

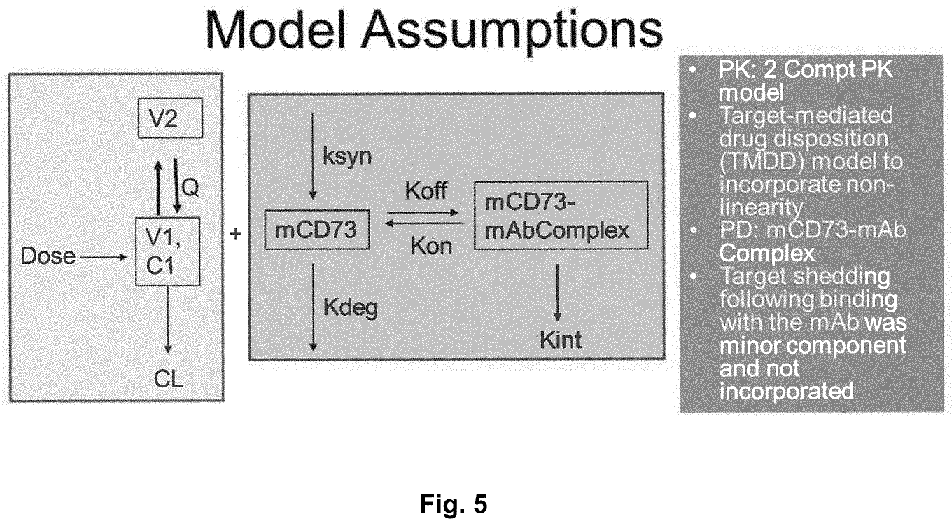

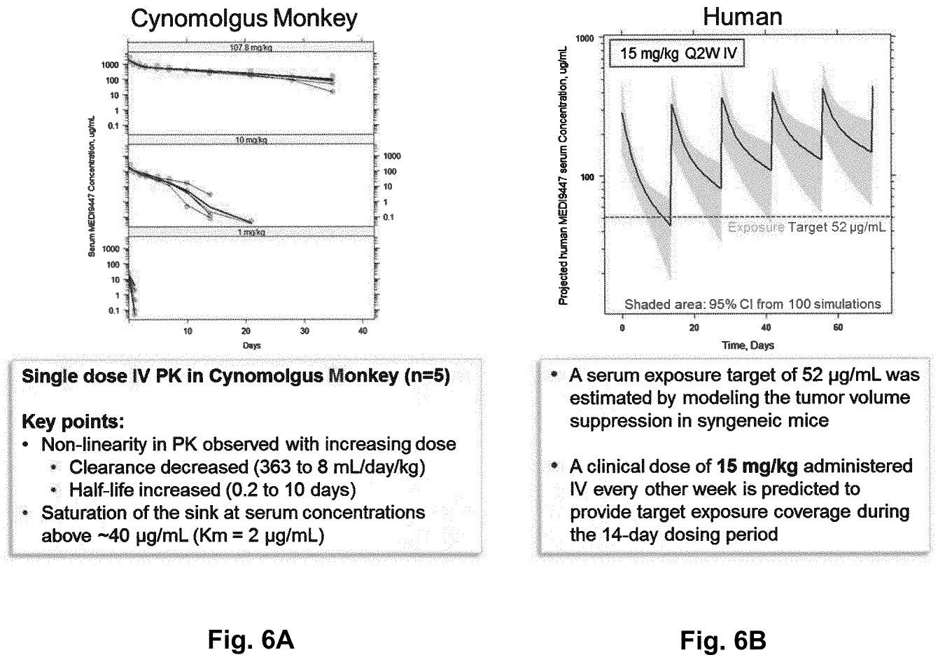

The endogenous pathogen response was also investigated by endogenous pathogen verification. Endogenous pathogen antibodies were measured using chemiluminescent ELISA on days 1, 8, 14, 15, and 29 and endogenous pathogen T cell IFN-γ ELISPOT was measured on days −14 and 29. LCV (monkey homologue of EBV) was detectable at low levels in most cynomolgus monkeys (FIG. 4). IgG titers to EBV gp125 and CMV gB were detectable but did not vary with oleclumab treatment. T cell responses to EBV BZLF1 and inactivated human CMV were detectable but did not vary consistently with oleclumab treatment

### Example 3: Oleclumab Monotherapy and in Combination with Durvalumab in Solid Tumor Treatment

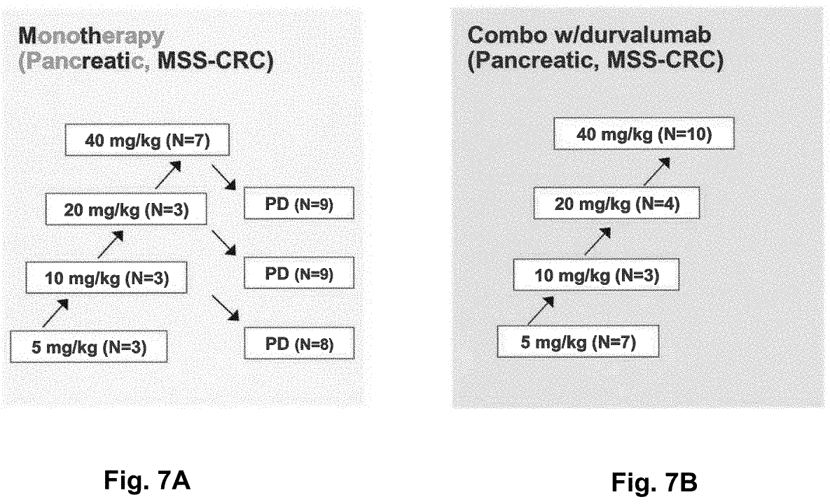

The study was a first-time-in-human (FTIH), Phase 1, multicenter, open-label, dose-escalation, and dose-expansion study of oleclumab administered as a single agent or in combination with durvalumab in adult subjects to evaluate the safety, tolerability, PK, immunogenicity, pharmacodynamics, and preliminary antitumor activity in adult subjects with selected advanced solid tumors. The study flow diagrams for dose escalation and dose expansion for this part of the study are illustrated in FIGS. 7A and 7B. The following abbreviations and legends are used to describe the study flow diagram DLT=dose-limiting toxicity; MSS-CRC=microsatellite stable CRC.

Safety was assessed by the presence of adverse events (AEs), serious adverse events (SAEs), DLTs, and changes from baseline in laboratory parameters, vital signs, and electrocardiogram results. The endpoints for assessment of antitumor activity included objective response (OR), disease control (DC), duration of response (DoR), progression-free survival (PFS), and overall survival (OS). RECIST v1.1 was used for assessment of tumor response. Pharmacokinetic parameters included, but were not limited to, maximum observed concentration, area under the concentration time curve, clearance, and terminal half-life. The endpoints for assessment of immunogenicity of oleclumab and durvalumab included the number and percentage of subjects who developed detectable anti-drug antibodies and the endpoints for assessment of pharmacodynamic activity included assessment of target expression (e.g., CD73, PD-L1) in tumor biopsy specimens.

### 1. Subjects

For the dose escalation arm the subject population included subjects ≥18 years of age, with histologically- or cytologically-confirmed colorectal adenocarcinoma (CRC) or pancreatic adenocarcinoma. Subjects with CRC or pancreatic adenocarcinoma must have received and progressed, were refractory, or were intolerant to standard therapy.

In the dose-escalation phase, subjects with CRC or pancreatic adenocarcinoma had not received more than five prior lines of therapy. Subjects with CRC enrolled in the dose-expansion phase had received at least two including regimens containing a fluoropyrimidine [e.g., 5-FU or capecitabine], oxaliplatin, and irinotecan unless contraindicated) but not more than four prior lines of systemic therapy in the metastatic setting and must not have defective DNA mismatch repair. Subjects with KRAS mutation (for example, exon 2, codon 12 or 13) were allowed.

Subjects with pancreatic adenocarcinoma enrolled in the dose-expansion phase must have received one but not more than two prior lines of systemic therapy in the metastatic setting. The first twenty subjects with CRC and pancreatic adenocarcinoma in the expansion phase must have had positive CD73 expression by IHC on at least 10% of tumor cells with weak, moderate, or strong staining; or a combination of such staining.

All subjects were required to have at least 1 lesion that was measurable using RECIST guidelines, an Eastern Cooperative Oncology Group (ECOG) score of 0 or 1, as well as adequate organ function. Adequate organ function was defined as: absolute neutrophil count ≥1,500/mm³; platelet count ≥75,000/mm³; Prothrombin time-international normalized ratio and partial thromboplastin time ≤1.5×ULN; hemoglobin ≥9.0 g/dL; creatinine clearance or 24-hour urine CrCl>50 mL/min as determined by the Cockcroft-Gault formula; total bilirubin ≥1.5×ULN except in the case of subjects with documented or suspected Gilbert's disease (for these subjects, bilirubin must be ≤3×ULN); aspartate aminotransferase (AST) and alanine aminotransferase (ALT) ≤2.5× upper limit of normal (ULN) (AST/ALT can be up to 5×ULN in the presence of liver metastasis, but cannot be associated with concurrent elevated bilirubin); potassium, sodium, magnesium, and calcium (corrected for serum albumin) ≤Grade 1 or within the institutional ranges of normal; and Albumin ≥3.0 g/dL.

Subjects were excluded from participation in the study if administered prior treatment with a TNFRSF agonist, received prior therapy with regimens containing CTLA-4, PD-L1, or PD-1 antagonists for subjects with CRC or pancreatic adenocarcinoma, required the use of additional immunosuppression other than corticosteroids for the management of an AE, experienced recurrence of an AE if re-challenged, and currently required maintenance doses of >10 mg prednisone or equivalent per day, received any conventional or investigational anticancer therapy within 28 days prior to the first dose of oleclumab within 14 days of the first dose of oleclumab, or received any concurrent chemotherapy, immunotherapy or biologic or hormonal therapy for cancer treatment.

### 2. Dose-Escalation Phase

The dose-escalation phase of the study consisted of 2 arms: (i) ascending dose levels of oleclumab monotherapy and (ii) ascending dose levels of oleclumab in combination with a single dose level of durvalumab, both administered in subjects with advanced CRC or pancreatic adenocarcinoma.

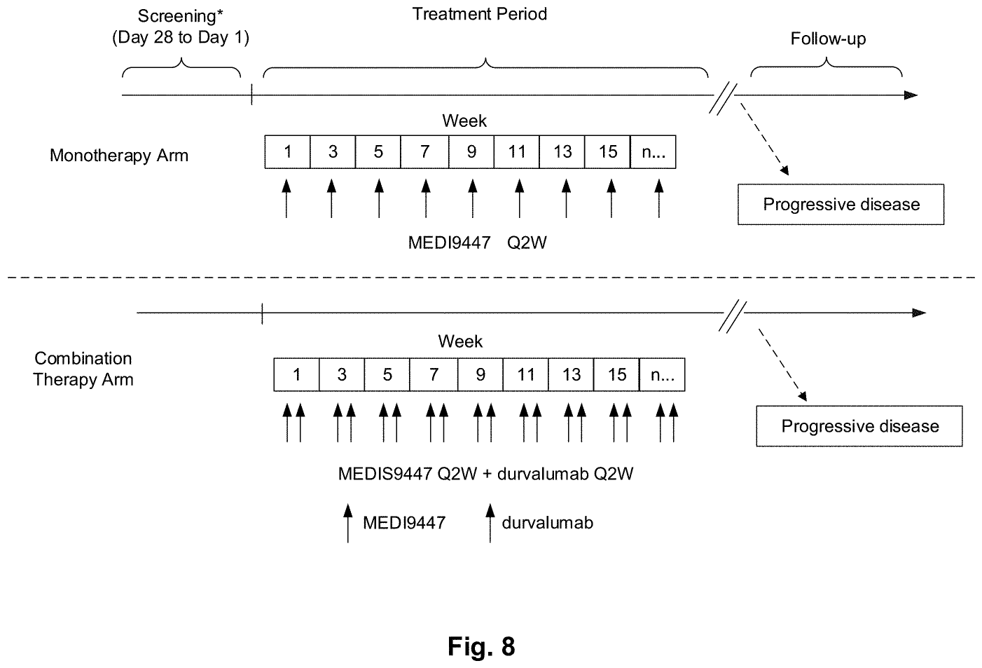

In the oleclumab monotherapy dose-escalation arm, sequential cohorts of 3 to 6 subjects each received 1 of 4 dose levels of oleclumab (5, 10, 20, or 40 mg/kg) via IV infusion Q2W unless the maximum tolerated dose (MTD) was reached before all dose-escalation cohorts were completed (FIG. 8). In the oleclumab/durvalumab combination therapy dose-escalation arm, sequential cohorts of 3 to 6 subjects each received 1 of 4 dose levels of oleclumab (5, 10, 20, or 40 mg/kg) and a single dose level of 10 mg/kg durvalumab via IV infusion Q2W, unless the MTD was reached before all dose-escalation cohorts were completed (FIG. 8). If the MTD of oleclumab was exceeded at the 5 mg/kg dose level in the monotherapy or combination therapy arm, a lower dose level of 2 mg/kg oleclumab was explored in that arm.

Oleclumab dose selection was based on nonclinical data and clinical safety margins based on nonclinical safety data as described above for mice and monkeys. The pharmacologically driven starting dose level of 5 mg/kg oleclumab was anticipated to have 98% to 91% saturation of CD73 in the first dosing interval (peak and trough, respectively). At the starting dose level of 5 mg/kg, cynomolgus monkey toxicity study provided safety margins of 19-fold (human equivalent dose [HED]-based), 73-fold (Cmax-based) and 70-fold (AUC-based). The dose-escalation scheme in this study was designed to achieve higher and more sustained suppression of CD73 target, while maintaining adequate safety margins. At the highest dose of 40 mg/kg, the cynomolgus monkey toxicity study provided safety margins of 2-fold (HED-based), 8-fold (Cmax-based), and 8-fold (AUC-based).

The dose level and treatment schedule for durvalumab (10 mg/kg Q2W) was based on a safe dose established in a Phase 1/2 study to evaluate the safety, tolerability, and PK of durvalumab in subjects with advanced solid tumors.

The proposed initial oleclumab and durvalumab combination dose level 1 utilized a dose level of durvalumab shown to have an acceptable safety profile (10 mg/kg IV Q2W) with a dose level of oleclumab (5 mg/kg IV Q2W) that was not anticipated to provide maximal inhibition of CD73 throughout the interval of dosing. If the MTD was exceeded prior to the proposed maximal combination doses of 40 mg/kg oleclumab and 10 mg/kg durvalumab, then 3 mg/kg durvalumab was utilized to further explore combination dosing.

The dose-escalation phase was executed on a 3+3 design. A minimum of three subjects were enrolled in each dose-level cohort, with administration of the first dose of investigational product staggered by a minimum of 24 hours between the first and second subjects treated in each dose-level cohort. If no dose limiting toxicities (DLTs) were observed in the first 3 subjects during the DLT-evaluation period) and all available safety data were reviewed by a study-specific dose escalation committee, dose-escalation continued to the next higher dose cohort. If 1 of 3 subjects in a dose-level cohort experienced a DLT, that dose level cohort was expanded to a total of 6 subjects. If no more than 1 of 6 subjects in the dose-level cohort experienced a DLT, dose-escalation continued to the next higher dose-level cohort. Six subjects were enrolled in the highest dose-level cohort that did not exceed the MTD.

In the oleclumab/durvalumab combination arm, if the MTD was exceeded at any oleclumab dose level, then an additional cohort of oleclumab with 3 mg/kg durvalumab Q2W was explored. The first cohort of 3 to 6 subjects received oleclumab at the same dose level and treatment schedule that exceeded the MTD but now with 3 mg/kg durvalumab via IV infusion Q2W. If the MTD was not exceeded and provided this was not the highest monotherapy oleclumab protocol-defined dose, additional sequential cohorts of 3 to 6 subjects each were enrolled according to the aforementioned oleclumab dose levels following either a Q2W or Q4W treatment schedule in combination with 3 mg/kg durvalumab Q2W. At completion of the combination dose-escalation, if the MTD was not exceeded with 10 mg/kg durvalumab Q2W, an alternate treatment schedule of oleclumab at the highest dose level that did not exceeded the MTD on either a Q2W or Q4W treatment schedule was explored in combination with 20 mg/kg durvalumab Q4W.

### 3. Results

Among the 42 efficacy evaluable subjects in the oleclumab 40 mg/kg+durvalumab 10 mg/kg dose-expansion phase, the overall objective response rate (ORR; confirmed and unconfirmed) was 7.1% (95% confidence interval [CI]: 1.5%, 19.5%). The ORRs (confirmed and unconfirmed) in the MSS-CRC (n=21) and pancreatic adenocarcinoma (n=20) cohorts were 4.8% (95% CI: 0.1%, 23.8%) and 10.0% (95% CI: 1.2%, 31.7%), respectively. The overall disease control rate (DCR; 8 weeks) in the dose-expansion phase was 16.7% (95% CI; 7.0%, 31.4%). The DCRs in the MSS-CRC and pancreatic adenocarcinoma cohorts were 14.3% (95% CI: 3.0%, 36.3%) and 20.0% (95% CI: 5.7%, 43.7%), respectively.

The following PK data is based on a total of 97 subjects following treatment with oleclumab 5 to 40 mg/kg Q2W administered either as monotherapy (n=40) or in combination with durvalumab at 10 mg/kg Q2W (n=57). Oleclumab appeared to exhibit a nonlinear PK at the lowest dose of oleclumab 5 mg/kg and exhibited linear PK at doses of oleclumab 10 mg/kg and higher in both monotherapy and combination therapy cohorts. Serum exposures were similar when oleclumab was administered either alone or in combination with durvalumab. The PK exposures (trough plasma concentration [Ctrough]) increased in a more than proportional manner from oleclumab 5 to 10 mg/kg and in an approximately dose proportional manner from oleclumab 10 to 40 mg/kg. Accumulation of oleclumab was observed following repeated dosing; the mean accumulation ratio ranged from 1.15 to 1.46 for Cₘₐₓ and from 1.68 to 10.7 for C_{trough}.

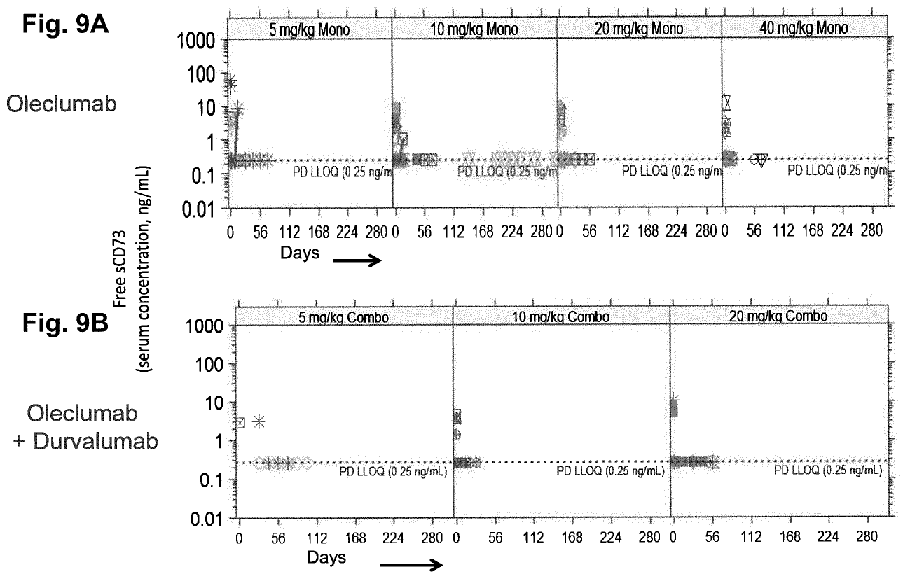

Complete suppression of free soluble CD73 was observed following administration of oleclumab either as monotherapy or in combination therapy. Free serum soluble CD73 levels were below the limit of detection (0.25 ng/mL) in the majority of subjects at all times after the first dose of oleclumab. (FIG. 9).

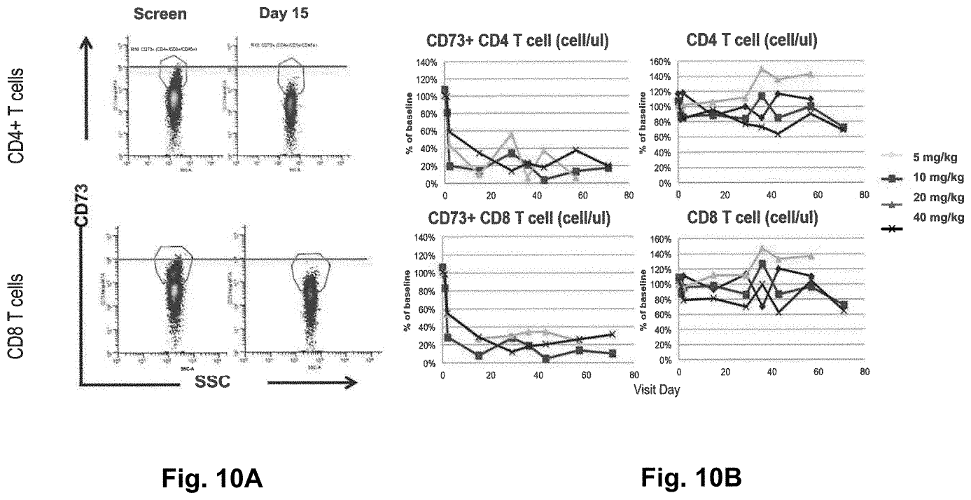

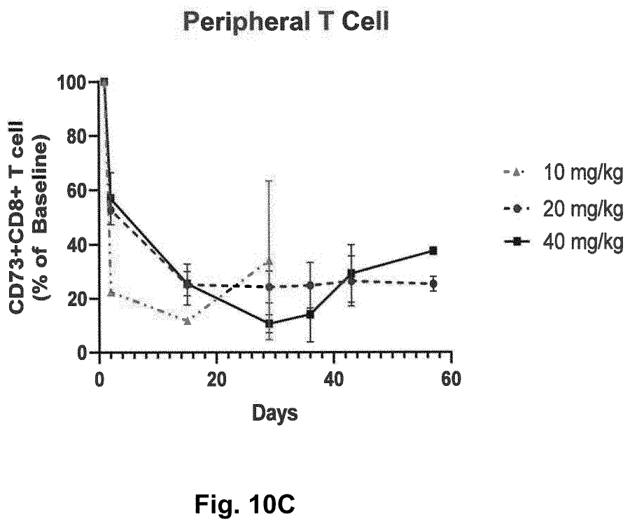

Oleclumab decreased CD73 surface expression as measured by mean fluorescence intensity (MFI) (FIG. 10A) and percent CD73+CD4 and CD8 cells (FIG. 10B) in peripheral T cells across all doses without a concomitant decrease in total CD4 and CD8 cells (FIG. 10C).

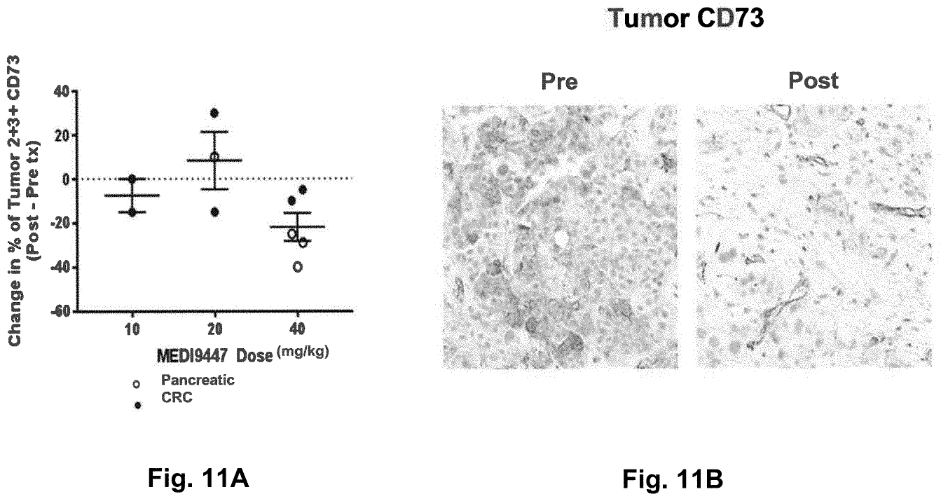

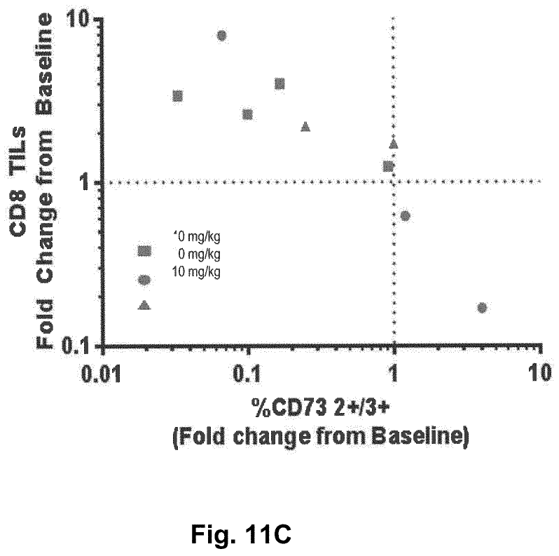

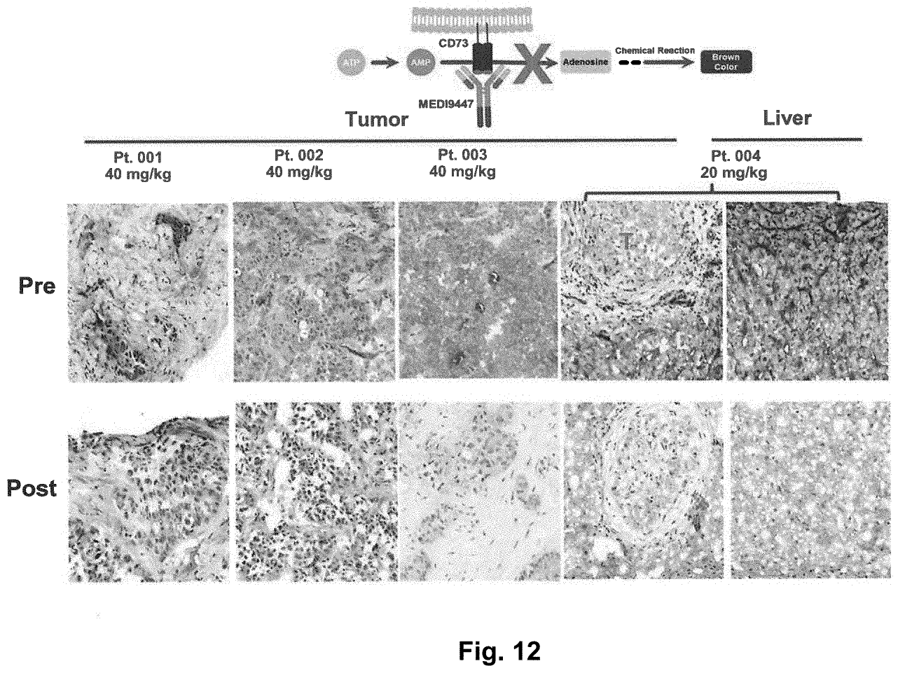

Treatment with oleclumab in dose escalation led to a decrease in CD73 staining tumor cells by IHC at the 40 mg/kg dose 20 days after treatment initiation (FIGS. 11A and 11B). Treatment with oleclumab alone decreased tumoral CD73 expression in 5/9 patients who expressed >5% 2+/3+ CD73 at baseline while increasing CD8+ TILs in all 5 samples (FIG. 11C). Oleclumab also inhibited CD73 enzymatic activity in tumor microenvironment (FIG. 12).

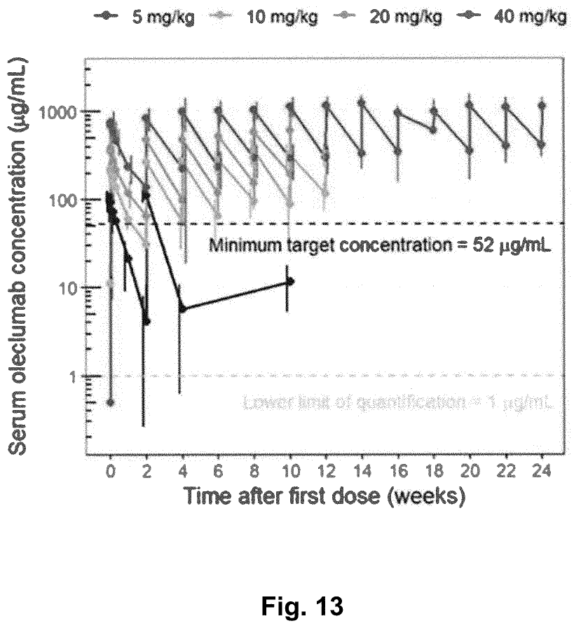

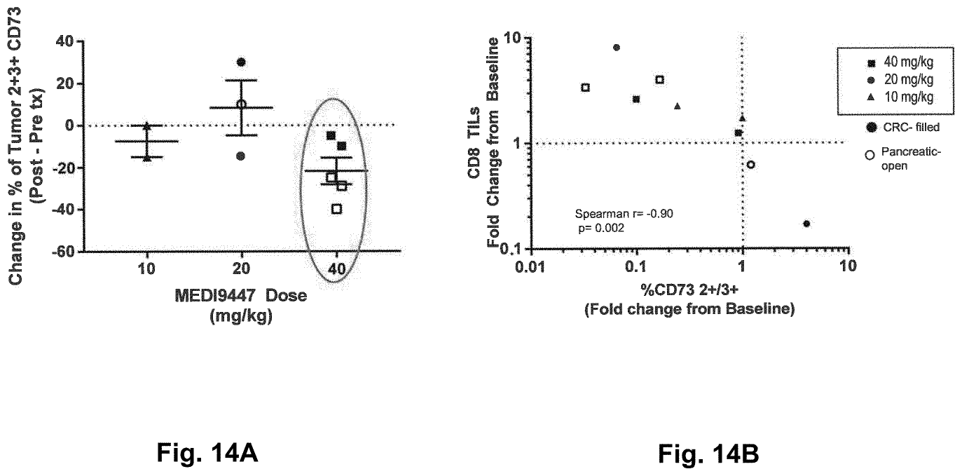

Linear PK was seen at ≥10 mg/kg doing of oleclumab. Based on these results, serum concentrations above 40 μg/mL is expected to saturate >99% CD73, and the estimated effective half-life in the dosing duration was determined to be ˜13 days (FIG. 13). No ADAs were detected. Oleclumab also demonstrated evidence of PD effect (FIG. 14). Decrease in CD73 at 40 mg/kg of oleclumab and was associated with increase in CD8+ T cells in patients >5% 2+/3+ CD73 at baseline. Based on analysis of safety, pharmacokinetics (PK), pharmacodynamics (PD) and preliminary efficacy, a recommended Phase 2 dose of oleclumab 40 mg/kg Q2W was selected.

### Example 4: Oleclumab Combination with Durvalumab; Dose-Expansion Phase

### 1. Subjects

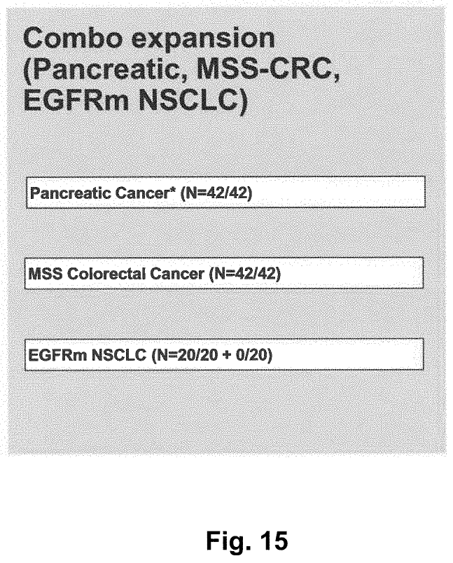

A dose-expansion study of oleclumab administered in combination with durvalumab was conducted in adult subjects to evaluate the safety, tolerability, PK, immunogenicity, pharmacodynamics, and preliminary antitumor activity in adult subjects with selected advanced solid tumors. The study flow diagram for the combo dose expansion is illustrated in FIG. 15. The following abbreviations and legends are used to describe the study flow diagram; MAD=maximum administered dose; MTD=maximum tolerated dose; EGFRm=epidermal growth factor receptor mutant; NSCLC=non-small cell lung cancer.

Subjects with NSCLC enrolled in dose-expansion phase must have had EGFR mutation known to be associated with EGFR TKI sensitivity (including G719X, exon 19 deletion, L858R, L861Q) and must have received at least one but not more than 4 prior lines of therapy (including investigational therapy) in the metastatic setting, must have received an approved EGFR TKI and then clinically or radiologically progressed or were intolerant.

Subjects were excluded from participation in the study if administered prior treatment with a TNFRSF agonist, had prior exposure to any investigational immunotherapy or receipt of an EGFR TKI, received prior therapy with regimens containing CTLA-4, PD-L1, required the use of additional immunosuppression other than corticosteroids for the management of an AE, experienced recurrence of an AE if re-challenged, and currently required maintenance doses of >10 mg prednisone or equivalent per day, received any conventional or investigational anticancer therapy within 28 days prior to the first dose of oleclumab within 14 days of the first dose of oleclumab, or received any concurrent chemotherapy, immunotherapy or biologic or hormonal therapy for cancer treatment.

Dose-expansion of oleclumab/durvalumab combination therapy was initiated once the MTD or MAD was established in the combination therapy arm of the dose-escalation phase. The combination therapy dose-expansion phase included the following three tumor-specific cohorts: a) up to 100 subjects with previously treated MSS-CRC, b) up to 100 subjects with previously treated pancreatic adenocarcinoma, and c) up to 40 subjects with previously treated EGFRm NSCLC.

The selection of dose level and treatment schedule was based on consideration of PK, safety, and comparative pharmacodynamic effects as a function of dose level and treatment schedule, with the limitation that the dose level would not exceed the applicable MTD or the MAD.

For each of the MSS-CRC, pancreatic adenocarcinoma, and EGFRm NSCLC dose-expansion cohorts, an interim analysis was performed when the first 20 subjects were enrolled and followed for at least 16 weeks. For each of the MSS-CRC and pancreatic adenocarcinoma cohorts, a second interim analysis was performed after 40 subjects were enrolled and followed for at least 16 weeks.

All subjects were evaluated for antitumor activity on a regular basis. Assessment of antitumor activity was conducted using objective response (OR), disease control (DC), duration of response (DoR), progression-free survival (PFS), and overall survival (OS). RECIST v1.1 guidelines were used for assessment of tumor response. All subjects will be followed for survival until the end of study.

### 3. Results

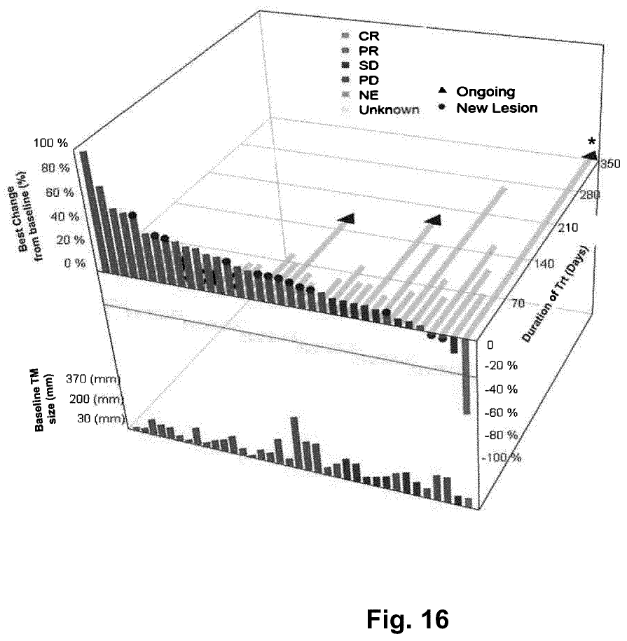

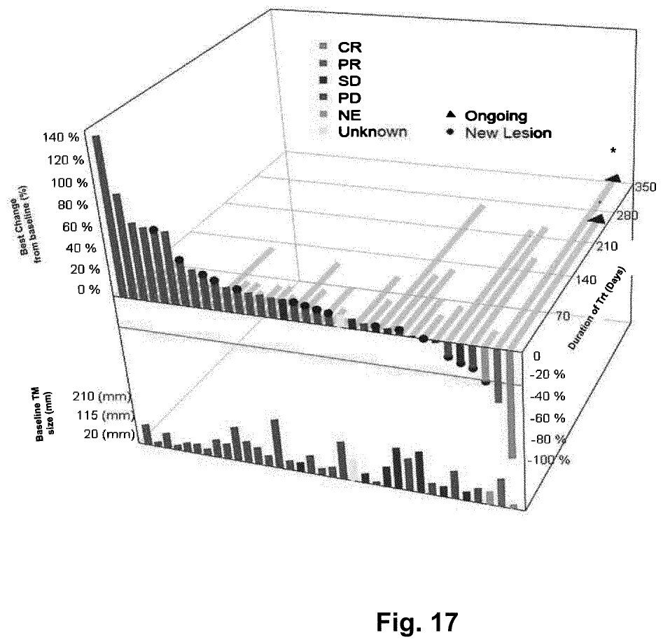

Among the 42 efficacy evaluable subjects in the oleclumab 40 mg/kg+durvalumab 10 mg/kg dose-expansion phase, the overall objective response rate (ORR; confirmed and unconfirmed) was 7.1% (95% confidence interval [CI]: 1.5%, 19.5%). The confirmed ORRs in the MSS-CRC (n=41) and pancreatic adenocarcinoma (n=41) cohorts were 2.4% (95% CI: 0.1%, 23.8%) and 7.3% (unconfirmed) and 4.9% (confirmed) (95% CI: 1.2%, 31.7%), respectively. See FIGS. 16 and 17. The overall disease control rate (DCR; 8 weeks) in the dose-expansion phase was 16.7% (95% CI; 7.0%, 31.4%). The DCRs in the MSS-CRC and pancreatic adenocarcinoma cohorts were 14.3% (95% CI: 3.0%, 36.3%) and 20.0% (95% CI: 5.7%, 43.7%), respectively.

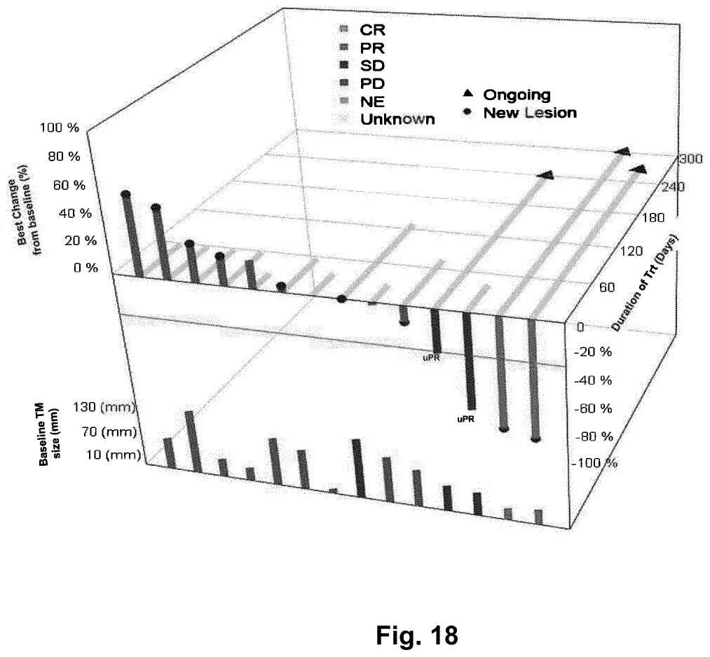

The confirmed ORRs in the EGFRm NSCLC (n=20) was 20% for all patients, as compared to 9.8% when durvalumab was administered as a monotherapy (see ATLANTIC trial; Clinictrials.gov No. NCT02087423) (FIG. 18).

### Example 5: Oleclumab Treatment with or without Durvalumab in Combination with Chemotherapy in Subjects with Metastatic Pancreatic Ductal Adenocarcinoma

The study is a Phase 1b/2, multicenter, open-label, dose-escalation and dose-expansion study to assess the safety, preliminary antitumor activity, immunogenicity, and PK of oleclumab with or without durvalumab in combination with chemotherapy administered in subjects with metastatic pancreatic ductal adenocarcinoma (PDAC). Subjects with previously untreated metastatic PDAC (1 L metastatic PDAC) were enrolled in Cohort A. Subjects with metastatic PDAC previously treated with gemcitabine-based chemotherapy (without exposure to 5-FU, capecitabine, or oxaliplatin; 2 L metastatic PDAC) were enrolled in Cohort B. The study consists of 2 parts, dose escalation (Part 1) and dose expansion (Part 2).

Up to approximately 204 subjects were enrolled in this study: up to 24 subjects in Part 1 (dose escalation) and up to 180 subjects in Part 2 (dose expansion). All subjects in both cohorts were treated until disease progression (and the treatment criteria in the setting of progressive disease [PD] were not met), intolerable toxicity, withdrawal of subject consent, or another discontinuation criterion is met.

Safety was assessed by the presence of adverse events (AEs), serious adverse events (SAEs), DLTs, and changes from baseline in laboratory parameters, vital signs, and electrocardiogram results. The endpoints for assessment of antitumor activity included objective response (OR), disease control (DC), duration of response (DoR), progression-free survival (PFS), and overall survival (OS). RECIST v1.1 was used for assessment of tumor response. Pharmacokinetic parameters included, but were not limited to, maximum observed concentration (Cₘₐₓ), time to reach Cₘₐₓ (tₘₐₓ), AUC, clearance, apparent volume of distribution (V_{d}), and terminal half-life (t_{1/2}). The development of anti-drug antibody (ADA) and its potential effect on safety, pharmacodynamics, PK, and antitumor activity were also assessed.

### 1. Subjects

Subjects in this study included adult subjects ≥18 years of age with histologically or cytologically confirmed pancreatic adenocarcinoma. Subjects with previously untreated metastatic PDAC (1 L metastatic PDAC) were enrolled in Cohort A. Subjects with metastatic PDAC previously treated with gemcitabine-based chemotherapy (without exposure to 5-FU, capecitabine, or oxaliplatin [if considered a line of therapy]; 2 L metastatic PDAC) were enrolled in Cohort B.

All subjects were required to have at least 1 lesion that was measurable using RECIST guidelines, an Eastern Cooperative Oncology Group (ECOG) score of 0 or 1, as well as adequate organ function. Adequate organ function is defined as: absolute neutrophil count ≥1,500μ/L; platelet count ≥100,000μ/L; hemoglobin ≥9.0 g/dL; creatinine clearance >40 mL/min; total bilirubin ≤1.5×ULN except in the case of subjects with documented or suspected Gilbert's disease (for these subjects, bilirubin must be ≤3×ULN); aspartate aminotransferase (AST) and alanine aminotransferase (ALT) ≤2.5× upper limit of normal (ULN) (AST/ALT can be up to 5×ULN in the presence of liver metastasis; and Albumin ≥3.0 g/dL.

Subjects were excluded from participation in the study if administered any conventional or investigational anticancer therapy within 21 days or palliative radiotherapy within 14 days prior to the scheduled first dose of study treatment or prior treatment with any immune-mediated therapy.

### 2. Dose Escalation Phase

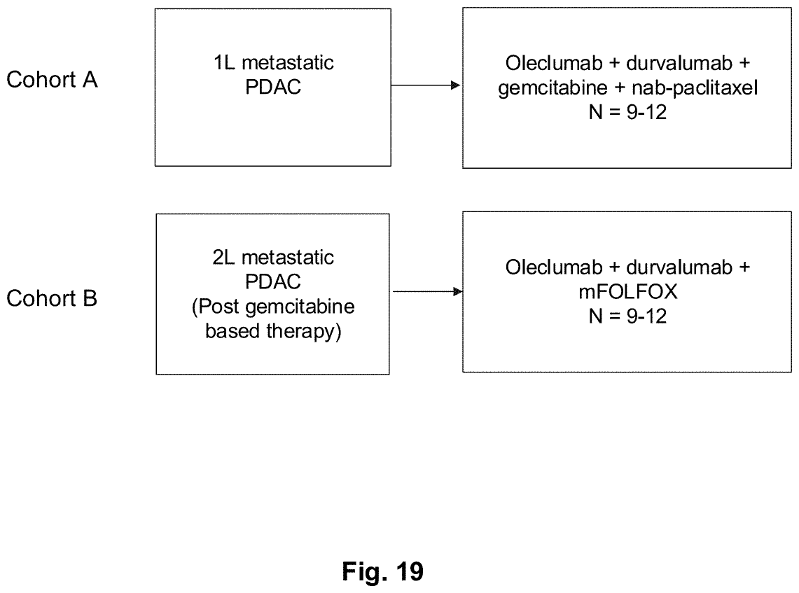

During Part 1, dose escalation of oleclumab was performed in combination with durvalumab and chemotherapy (gemcitabine+nab-paclitaxel for subjects with 1 L metastatic PDAC [Cohort A]; modified regimen of leucovorin, 5-fluorouracil, and oxaliplatin (mFOLFOX) for subjects with 2 L metastatic PDAC [Cohort B]) to determine either the maximum tolerated dose (MTD) or the highest protocol-defined dose for each regimen (See FIG. 19). Up to 24 subjects were enrolled in Part 1 (dose escalation): 9-12 subjects with 1 L metastatic PDAC were enrolled in Cohort A and 9-12 subjects with 2 L metastatic PDAC were enrolled in Cohort B and treated with increasing dose levels of oleclumab.

Dose escalation began with enrollment of at least 3 subjects (and up to 6 subjects) at dose level 1 (1500 mg IV Q2W×4 then Q4W). Subjects were monitored for DLTs. If no DLTs were observed in a cohort of 3 to 6 evaluable subjects, then dose escalation to the next higher dose cohort was permitted after review of all available safety data. If 1 subject in a dose-level cohort of 3 or more evaluable subjects experiences a DLT, that dose-level cohort was expanded to a total of 6 subjects. If no more than 1 of 6 subjects in the dose-level cohort experiences a DLT, dose escalation continued to the next higher dose-level cohort. If ≥2 subjects in a dose-level cohort experience a DLT, the MTD was exceeded, and no further subjects were enrolled into that dose-level cohort. If this occurred, the preceding dose-level cohort was evaluated for the MTD and a total of 6 subjects were treated at the preceding dose level if not already expanded. If ≤1 of 6 subjects experienced a DLT at the preceding dose level, then this dose level was the MTD. If the MTD was exceeded at the starting dose level, then a lower dose level of oleclumab 750 mg (dose level −1) could be evaluated.

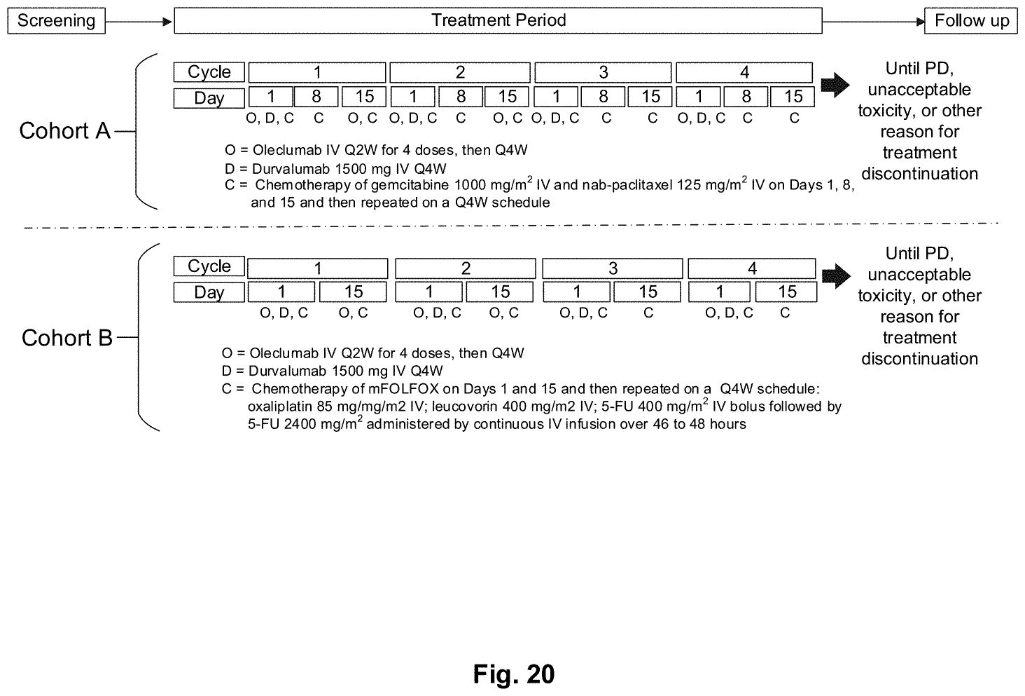

As shown in FIG. 20 and outlined below, during the dose escalation phase, patients received the following treatment:

### Cohort A:

Oleclumab at one of 3 dose levels (750 mg, 1500 mg, or 3000 mg) IV Q2W for 4 doses, then every 4 weeks (Q4W) and
Durvalumab 1500 mg IV Q4W and
Gemcitabine 1000 mg/m2 IV and nab-paclitaxel 125 mg/m2 IV on Days 1, 8, and 15 and then repeated on a Q4W schedule

### Cohort B:

Oleclumab at one of 3 dose levels (750 mg, 1500 mg, or 3000 mg) IV Q2W for 4 doses, then Q4W and
Durvalumab 1500 mg IV Q4W and
mFOLFOX on Days 1 and 15 and then repeated on a Q4W schedule: oxaliplatin 85 mg/m² IV; leucovorin 400 mg/m² IV; 5-FU 400 mg/m² IV bolus followed by 5-FU 2400 mg/m² administered by continuous IV infusion over 46 to 48 hours

TABLE 1
Oleclumab Dose Levels for Evaluation in Part 1 (Dose Escalation)

| | Dose Level −1 | Dose Level 1 | Dose Level 2 |
|---|---|---|---|
| Agents | N = 3-6 subjects | N = 3-6 subjects | N = 6 subjects |
| Oleclumab | 750 mg IV | 1500 mg IV | 3000 mg IV |
| (Cohorts A | Q2W × 4 | Q2W × 4 | Q2W × 4 |
| and B) | then Q4W | then Q4W | then Q4W |

### 3. Dose Expansion Phase

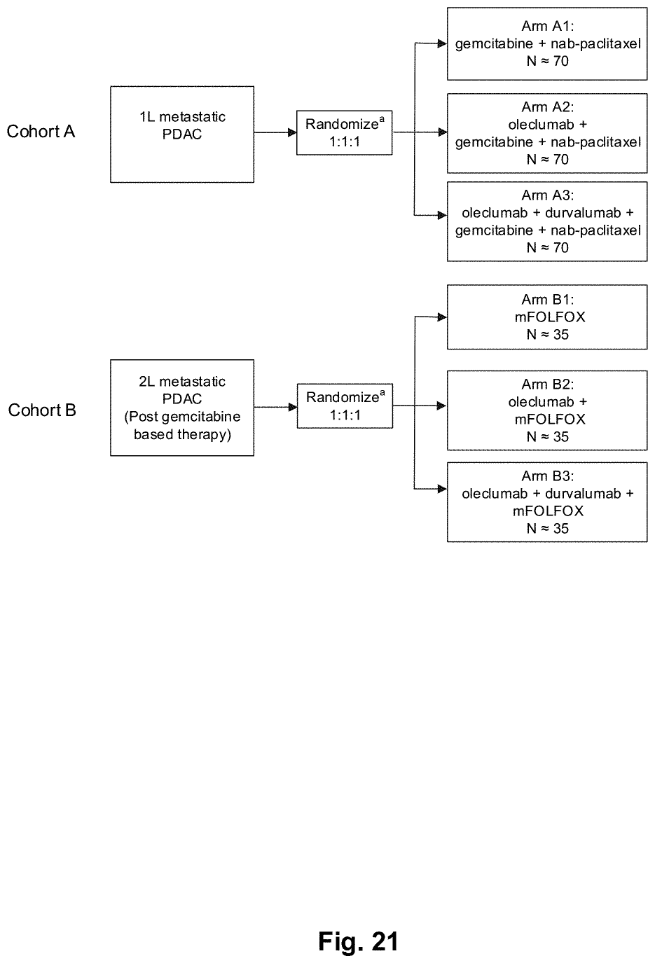

Once the recommended phase 2 dose (RP2D) for a cohort had been identified, enrollment into Part 2 dose expansion proceeded as outlined in FIG. 21. During Part 2 (dose-expansion), the RP2D of oleclumab identified in Part 1 for each regimen was evaluated with or without durvalumab in combination with chemotherapy. Up to 180 subjects were enrolled in Part 2, with 30 subjects per treatment arm. Patients were stratified according to tumoral expression of CD73 by immunohistochemistry (IHC) and randomized to a treatment arm. Subjects in Cohort A (1 L metastatic PDAC) were randomized 1:1:1 to one of 3 treatment arms: gemcitabine and nab-paclitaxel (Arm A1); oleclumab+gemcitabine and nab-paclitaxel (Arm A2); or oleclumab+durvalumab+gemcitabine and nab-paclitaxel (Arm A3). Subjects in Cohort B (2 L metastatic PDAC) will be randomized 1:1:1 to one of 3 treatment arms: mFOLFOX (Arm B1); oleclumab+mFOLFOX (Arm B2); or oleclumab+durvalumab+mFOLFOX (Arm B3). There was no crossover between treatment arms.

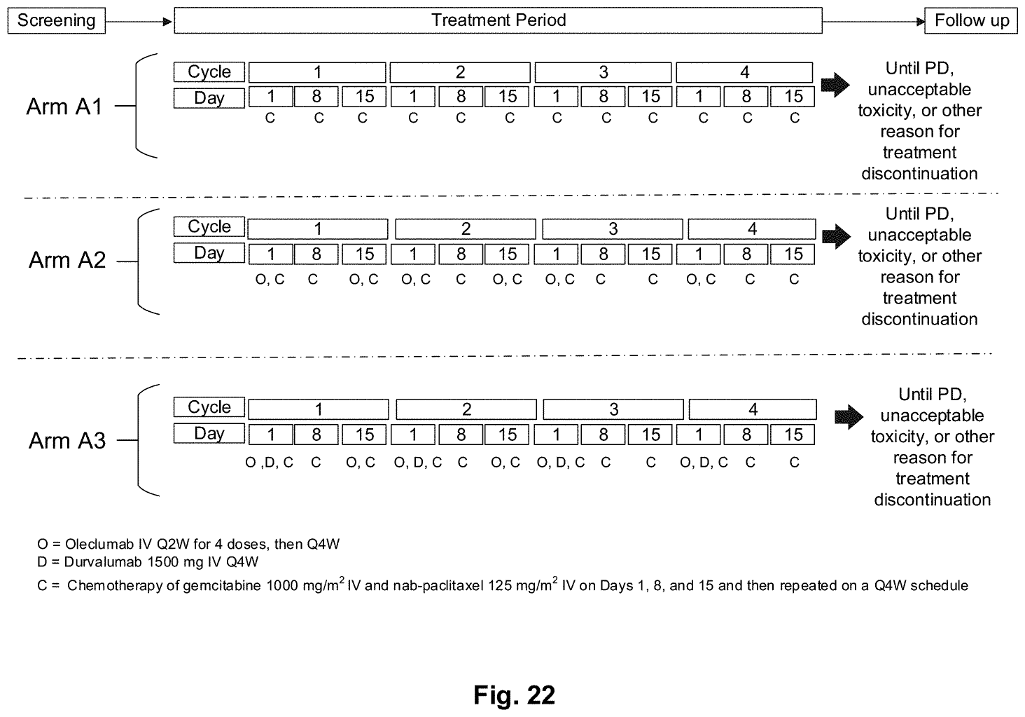

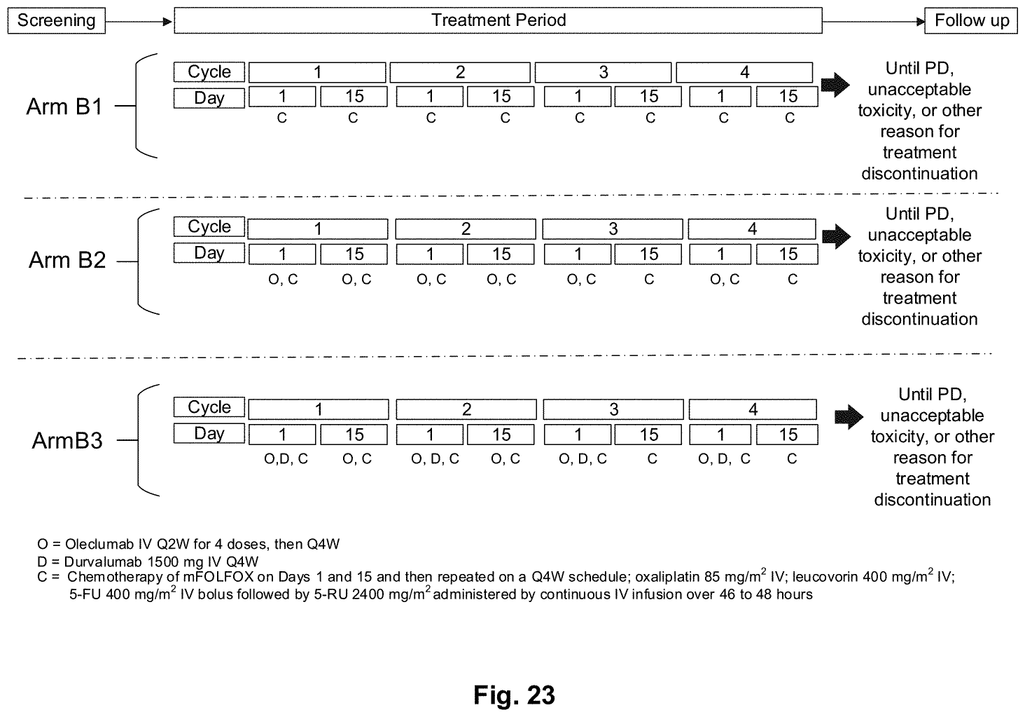

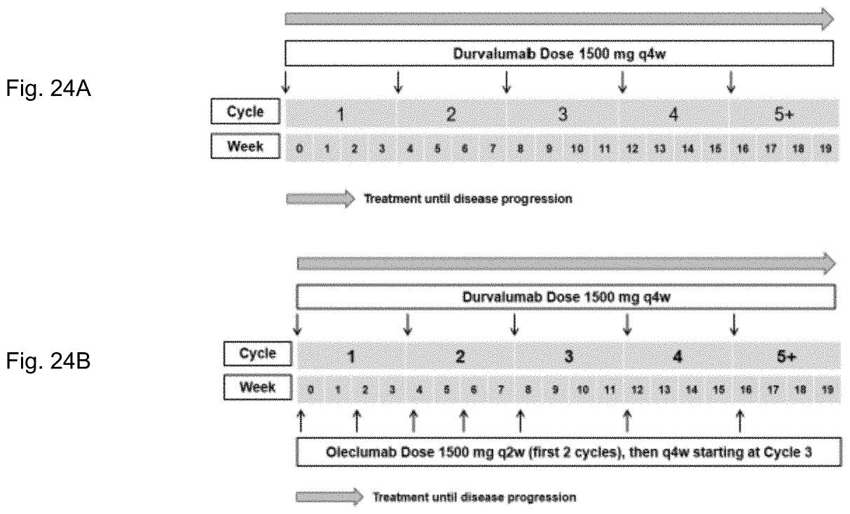

The dose level for oleclumab was determined during Part 1 (dose escalation). As shown in FIGS. 22 and 23 and outlined below, subjects in Cohorts A and B were randomized to receive the treatments as follows:

### Cohort A

Arm A1
   Gemcitabine 1000 mg/m² IV and nab-paclitaxel 125 mg/m² IV on Days 1, 8, and 15 and then repeated on a Q4W schedule
Arm A2
   Oleclumab IV Q2W for 4 doses, then Q4W and
   Gemcitabine 1000 mg/m² IV and nab-paclitaxel 125 mg/m² IV on Days 1, 8, and 15 and then repeated on a Q4W schedule
Arm A3
   Oleclumab IV Q2W for 4 doses, then Q4W and
   Durvalumab 1500 mg IV Q4W and
   Gemcitabine 1000 mg/m² IV and nab-paclitaxel 125 mg/m² IV on Days 1, 8, and 15 and then repeated on a Q4W schedule

### Cohort B

Arm B1
   mFOLFOX on Days 1 and 15 and then repeated on a Q4W schedule: Oxaliplatin 85 mg/m² IV; leucovorin 400 mg/m² IV; 5-FU 400 mg/m² IV bolus followed by 5-FU 2400 mg/m² administered by continuous IV infusion over 46 to 48 hours
Arm B2
   Oleclumab IV Q2W for 4 doses, then Q4W and
   mFOLFOX on Days 1 and 15 and then repeated on a Q4W schedule: Oxaliplatin 85 mg/m² IV; leucovorin 400 mg/m² IV; 5-FU 400 mg/m² IV bolus followed by 5-FU 2400 mg/m² administered by continuous IV infusion over 46 to 48 hours
Arm B3
   Oleclumab IV Q2W for 4 doses, then Q4W and
   Durvalumab 1500 mg IV Q4W and
   mFOLFOX on Days 1 and 15 and then repeated on a Q4W schedule: Oxaliplatin 85 mg/m² IV; leucovorin 400 mg/m² IV; 5-FU 400 mg/m² IV bolus followed by 5-FU 2400 mg/m² administered by continuous IV infusion over 46 to 48 hours.

### Example 6: Oleclumab and Durvalumab Treatment with or without Chemotherapy in Subjects with First-Line Stage IV Non-Small Cell Lung Cancer (NSCLC)

A Phase 1b open-label, multicenter study is performed to assess the efficacy (antitumor activity) and safety of oleclumab and durvalumab with or without chemotherapy in subjects with first-line stage IV non-small cell lung cancer (NSCLC). Subjects with high-PD-L1 (i.e., PD-L1 TC ≥50%) and low PD-L1 (i.e., PD-L1 TC<50%) were treated.

### 1. Subjects

Subjects in this study include adult subjects ≥18 years of age with histologically or cytologically documented Stage IV NSCLC not amendable to curative surgery or radiation with tumors lacking activating epidermal growth factor receptor (EGFR) mutations and anaplastic lymphoma kinase (ALK) fusions. Subjects have no prior chemotherapy or any other systemic therapy for Stage IV NSCLC. All subjects are required to have a World Health Organization (WHO)/Eastern Cooperative Oncology Group (ECOG) performance status of 0 or 1 at enrollment and treatment assignment. Subjects have no prior exposure to immune-mediated therapy including, excluding therapeutic anti-cancer vaccines.

Subjects were excluded from participation in the study if they received any prior chemotherapy or any other systemic therapy for Stage IV NSCLC; if they are receiving any concurrent chemotherapy, biologic, or hormonal therapy for cancer treatment; of if they received or are receiving any immunosuppressive medication within 28 days before the first administration of this study other than (i) intranasal, inhaled, or topical steroids, or local steroid injections (ii) systemic corticosteroids at physiologic doses note to exceed 10 mg/day of prednisone or its equivalent; and (iii) steroids as pre-medication for hypersensitivity reactions. Subjects are also excluded if they received radiation therapy unless it was (i) definitive radiation that had been administered at least 12 months prior to the date of progression to Stage IV disease, (ii) palliative radiation to brain, with associated criteria for stability or lack of symptoms, at least 4 weeks prior to the first study treatment dose, or (iii) palliative radiation to painful bony lesions (must comprise less than 30% of bone marrow) at least 2 weeks prior to the first study treatment dose.

### 2. Treatment

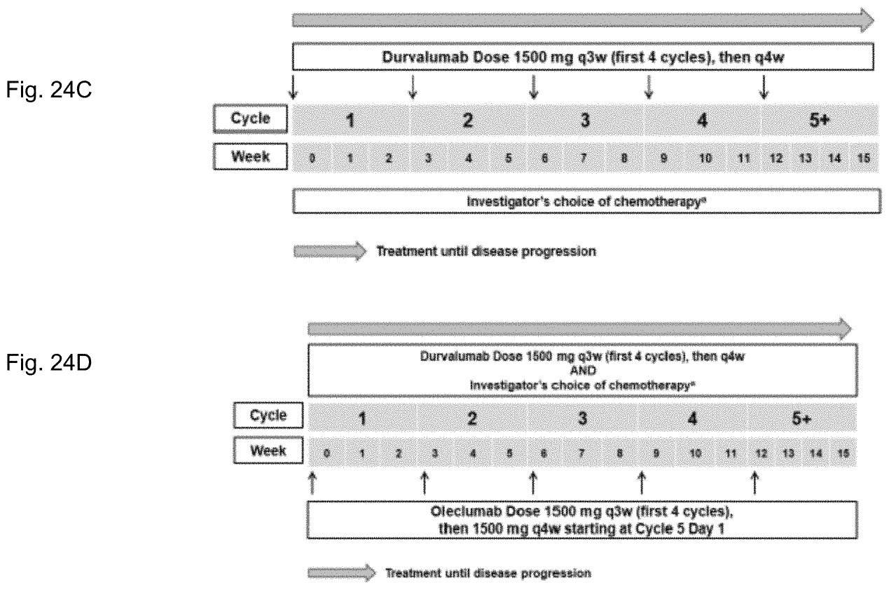

At least 30 patients were enrolled in each treatment arm as provided in Table 2 and shown in FIGS. 24A-24D. Patients with high-PD-L1 (i.e., PD-L1 expression on ≥50% of tumor cells) were enrolled in Cohort A, and patients with and low PD-L1 (i.e., PD-L1 expression on <50% of tumor cells) were enrolled in Cohort B.

TABLE 2
Oleclumab + Durvalumab ± Chemotherapy Treatment Groups

| Cohort | Arm | Treatment |
|---|---|---|
| A | A1 | Durvalumab |
| A | A3 | Durvalumab + oleclumab |
| B | B1 | Durvalumab + chemotherapy |
| B | B3 | Durvalumab + chemotherapy + oleclumab |

Chemotherapy is selected from: (a) nab-paclitaxel+carboplatin ((squamous and non-squamous patients); (b) gemcitabine+cisplatin (squamous patients only); (c) gemcitabine+carboplatin (squamous patients only); (d) pemetrexed+carboplatin (non-squamous patients only), and (e) pemetrexed+cisplatin (non-squamous patients only). Non-squamous patients who receive carboplatin/cisplatin+pemetrexed and who progress after 4 cycles of carboplatin/cisplatin+pemetrexed receive pemetrexed maintenance therapy, unless contraindicated. For Arm B1, pemetrexed maintenance therapy can be given either every three weeks (q3w) or every four weeks (q4w). Pemetrexed maintenance therapy can be given q4w for Arm B3.

Oleclumab, durvalumab, and chemotherapy were administered per the schedules in Table 3.

TABLE 3
Oleclumab + Durvalumab ± Chemotherapy
Administration Schedules

| Treatment | Cohort | Dose | | Schedule |
|---|---|---|---|---|
| Durvalumab | A | 1500 | mg^{a} | Q4W |
| Durvalumab | B | 1500 | mg^{a} | Q3W for the first 4 |
| | | | | cycles; then Q4W |
| | | | | starting at Cycle 5, |
| | | | | Day 1 |
| Oleclumab | A | 1500 | mg | Q2W for the first 2 |
| | | | | cycles (Day 1 and |
| | | | | Day 15 of Cycle 1 |
| | | | | and Cycle 2); then |
| | | | | Q4W starting at |
| | | | | Cycle 3 Day 1 |
| Oleclumab | B | 1500 | mg | Q3W for the first 4 |
| | | | | cycles; then Q4W |
| | | | | starting at Cycle 5, |
| | | | | Day 1 |
| Nab- | B | 100 | mg/m² | Days 1, 8, and 15 of |
| paclitaxel | | | | each 21-day cycle |
| Gemcitabine | B | 1000 or 1250 mg/m² | | Days 1 and 8 of each |
| | | | | 21-day cycle |
| Pemetrexed | B | 500 | mg/m² | Day 1 of each 21-day |
| | | | | cycle |
| Carboplatin | B | AUC 5 or 6 | | Day 1 of each 21-day |
| | | | | cycle |
| Cisplatin | B | 75 | mg/m² | Day 1 of each 21-day |
| | | | | cycle |

^{a}Weight-based dosing at 20 mg/kg administered if weight falls to ≤30 kg.

In the event that the initial dose level in Arm A3 and/or B3 is tolerated, a new treatment arm was opened at higher dose of oleclumab as shown in Table 4.

TABLE 4
Oleclumab Escalation

| | Dose Level | |
|---|---|---|
| Treatment | Initial dose (level 1) | Dose level 2 |
| Oleclumab - | 1500 mg iv Q2W first 2 | 3000 mg iv Q2W first 2 |
| Cohort A | cycles (D1 and D15 of | cycles, then Q4W starting |
| | C1 and C2), then Q4W | at C3D1 |
| | starting at C3D1 | |
| Oleclumab - | 1500 mg Q3W for the | 2250 mg Q3W for the |
| Cohort B | first 4 cycles, then | first 4 cycles, then |
| | 1500 mg Q4W starting | 3000 mg Q4W starting |
| | at C5D1 | at C5D1 |

Treatment continues until clinical progression or radiological progression occur. In arms including chemotherapy, chemotherapy were administered for 4 cycles or until progression of disease (PD) is observed, whichever occurs sooner (i.e., 4 cycles unless PD occurs prior to completion of the planned therapy).

### 3. Results

Adverse events, physical examinations, laboratory findings, and vital signs were assessed from all arms to demonstrate that oleclumab and durvalumab with or without chemotherapy are safe.

8 patients were dosed with durvalumab and oleclumab in Cohort A3 and 6 patients were dosed in the Cohort B3 safety run-in for the durvalumab+chemotherapy+oleclumab arm. Safety was assessed and doses were well tolerated. The Study Level Safety Review meeting agreed to escalate Cohort B3 from 1500 mg oleclumab to the 3000 mg dose as no DLTs were reported.

The overall response rates (ORR), progression-free survival times, and date of objective responses were evaluated in all arms to demonstrate that the combination of oleclumab and durvalumab is effective in the treatment of first-line stage IV non-small cell lung cancer (NSCLC) in patients with high-PD-L1 and that the combination of oleclumab, durvalumab, and chemotherapy is effective in the treatment of first-line stage IV in patients with low PD-L1.

### Example 7: Oleclumab and Durvalumab Treatment in Subjects with Locally Advanced, Unresectable, Stage III Non-Small Cell Lung Cancer

A Phase 2 open-label, multicenter study was performed to assess the efficacy (antitumor activity) and safety of oleclumab and durvalumab in subjects with locally advanced, unresectable, stage III non-small cell lung cancer (NSCLC).

### 1. Subjects

Subjects in this study included adult subjects at least 18 years of age (with a body weight of at least 35 kg) with locally advanced, unresectable, stage III NSCLC who have not progressed following definitive concurrent chemoradiotherapy (cCRT). Definitive radiotherapy refers to a total dose of ≥60 Gy at 1.8 Gy per fraction or bioequivalent dose. Concurrent chemotherapy refers to a platinum-based doublet. The final chemotherapy administration must end prior to, or concurrently with, the final dose of radiation.

All subjects were required to have had at least one previously irradiated tumor lesion that could be measured by RECIST v. 1.1, a life expectancy of at least 12 weeks, and an Eastern Cooperative Oncology Group (ECOG) Performance Status of 0 or 1.

Subjects were excluded from participation in the study if they had mixed small cell and non-small cell lung cancer histology. Subjects were also excluded from the study for use of immunosuppressive medications within 14 days before the first dose of study drug other than (i) intranasal, inhaled, or topical steroids, or local steroid injections (ii) systemic corticosteroids at physiologic doses note to exceed 10 mg/day of prednisone or its equivalent; and (iii) steroids as pre-medication for hypersensitivity reactions. Subjects were also excluded for any prior exposure to anti-PD-1, anti-PD-L1 or anti-cytotoxic T-lymphocyte associated antigen-4 (CTLA-4) antibody for the treatment of NSCLC.

### 2. Treatment

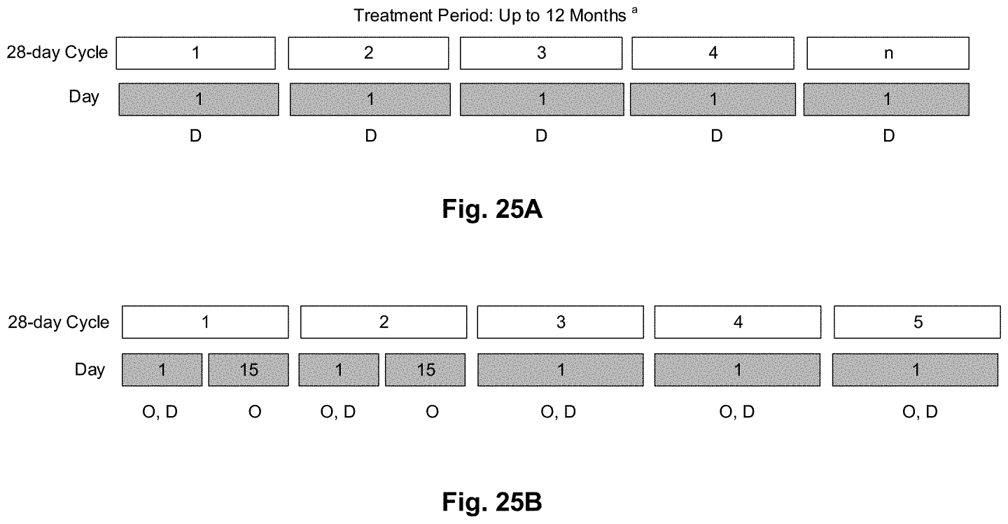

Patients initiated study treatment within 42 days from their last session of cCRT. Up to 60 subjects per treatment arm were randomized with equal ratios to durvalumab control and experimental arm. Patients in the control arm received 1500 mg durvalumab intravenously every 4 weeks (Q4W) for 12 months. See FIG. 25A. Patients in the experimental arm received (i) 1500 mg durvalumab intravenously every 4 weeks (Q4W) for 12 months plus (ii) 3000 mg oleclumab intravenously every 2 weeks (Q2W) for 2 months and then Q4W starting on Cycle 3, Day 1 for 10 months. See FIG. 25B.

Subjects were treated for up to 12 months, unless disease progression, unacceptable toxicity, or another reason (e.g., subject decision or noncompliance) for termination of treatment occurred.

### 3. Results

Adverse events, laboratory findings, electrocardiogram results, and vital signs are assessed to demonstrate that the combination of oleclumab and durvalumab is safe. The objective response (OR) per RECIST v. 1.1, duration of response (DoR), disease control (DC), progression-free survival (PFS) at 12 months, PFS per RECIST v. 1.1, and overall survival (OS) are evaluated to demonstrate that the combination of oleclumab and durvalumab is more effective in the treatment of Stage III NSCLC than durvalumab alone.

### Example 8: Oleclumab and Durvalumab Treatment in Subjects with Resectable, Early-Stage Non-Small Cell Lung Cancer

A Phase 2 open-label, multicenter study was performed to assess the efficacy (antitumor activity) and safety of oleclumab and durvalumab in subjects with resectable, early-stage non-small cell lung cancer (NSCLC).

### 1. Subjects

Subjects in this study included adult subjects at least 18 years of age (with a body weight of at least 35 kg) with cytologically and/or histologically documented NSCLC that was (a) Stage I (>2 cm) to IIIA (for subjects with N2 disease, only those with 1 single nodal station ≤3 cm were eligible) NSCLC according to the 8^{th} edition of American Joint Committee on Cancer staging classification; and (b) considered amenable to complete surgical resection. The subjects had not received any other therapy (chemotherapy, biologic, or radiotherapy) for this condition. All subjects had an Eastern Cooperative Oncology Group (ECOG) Performance Status of 0 or 1.

Subjects were excluded from participation in the study if they had mixed small cell and non-small cell lung cancer histology. Subjects were also excluded from the study as a result of participation in another interventional clinical study within 90 days prior to enrollment. Subjects were also excluded from the study for use of immunosuppressive medications within 14 days before the first dose of study drug other than (i) intranasal, inhaled, or topical steroids, or local steroid injections (ii) systemic corticosteroids at physiologic doses note to exceed 12 mg/day of prednisone or its equivalent; and (iii) steroids as pre-medication for hypersensitivity reactions.

Up to 40 subjects per treatment arm were enrolled.

### 2. Treatment

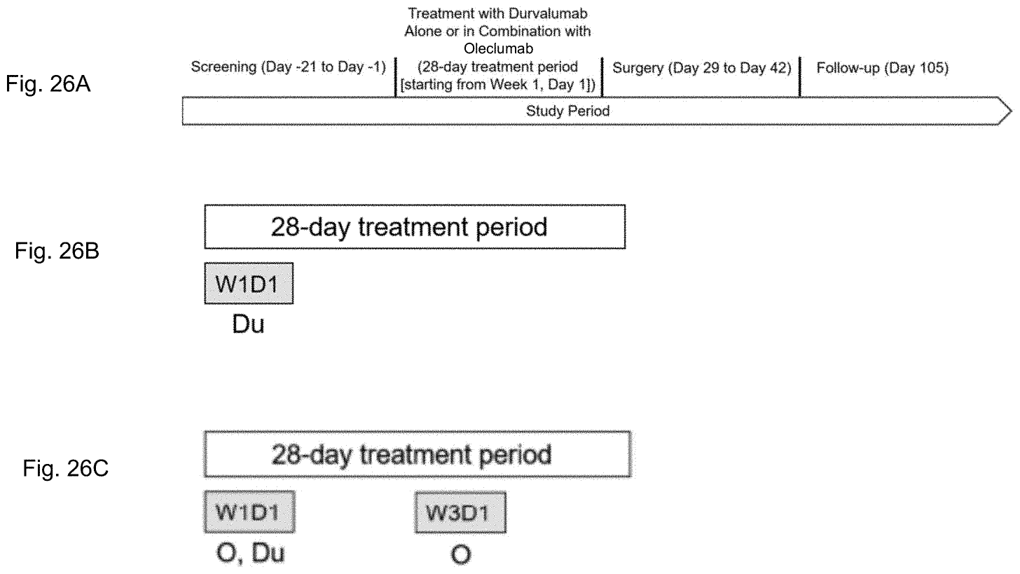

The treatment over the course of the study duration period is shown in FIG. 26A. Subjects were treated with either durvalumab monotherapy or a combination of durvalumab and oleclumab for up to 28 days. Treatment was discontinued upon disease progression, unacceptable toxicity, or another reason (e.g., subject decision or noncompliance). Subjects who received durvalumab monotherapy received 1500 mg durvalumab intravenously Q4W on Week 1, Day 1. (FIG. 26B.) Subjects who received combination therapy received 1500 mg durvalumab intravenously Q4W on Week 1, Day 1, plus 3000 mg oleclumab intravenously Q2W on Week 1, Day 1, and Week 3, Day 1. (FIG. 26C.)

The 28-day treatment period was followed by surgical resection. The surgical resection was within 14 days of the treatment period. After surgical resection, subjects were followed up to Day 105. If a subject received adjuvant chemotherapy or radiotherapy prior to Day 105, the subject came off study, and the end of study visit was scheduled prior to the start of adjuvant therapy.

### 3. Results

Pathological changes (e.g., major pathologic responses (MPRs)) are evaluated to demonstrate that the combination of oleclumab and durvalumab leads to a pathological response within the resected tumor specimen of early-stage NSCLC cancer patients. MPRs, pathological complete response (pCR), and best overall response (BOR) and ORR per RECIST v 1.1 are also evaluated to demonstrate that the combination of oleclumab and durvalumab has antitumor activity in resectable, early-stage NSCLC. Adverse events, laboratory findings, and vital signs are assessed to demonstrate that the combination of oleclumab and durvalumab is safe.

### Example 9: Oleclumab and Durvalumab in Combination with Chemotherapy and Bevacizumab Treatment in Subjects with Metastatic Microsatellite-Stable Colorectal Cancer

A Phase 1b/2 open-label, multicenter study was performed to assess the efficacy (antitumor activity) and safety of oleclumab and durvalumab in combination with chemotherapy and bevacizumab as a first-line (1 L) therapy in subjects with metastatic microsatellite-stable colorectal cancer (MSS-CRC). The study included two parts. Part 1 was a Phase 1b safety study, and Part 2 was a Phase 2 study of efficacy and safety.

### 1. Subjects

Subjects in this study included adult subjects at least 18 years of age (with a body weight of at least 35 kg) with metastatic MSS-CRC who had not received prior systemic treatment in the recurrent/metastatic setting (subjects treated with prior adjuvant chemotherapy or radio-chemotherapy were accepted as long as progression was not within 6 months of completing the adjuvant regimen). All subject had histological documentation of advanced or metastatic CRC and a documented mutation test during screening and confirmed tumor locations from disease assessment. Subjects must not have had defective DNA mismatch repair (MSI) as document by testing. Subjects had at least one lesion that was measurable by RECIST v1.1 (a previously irradiated lesion could be considered a target legion if the lesion was well defined, measurable per RECIST, and had clearly progressed during or after the most recent therapy). All subjects had an Eastern Cooperative Oncology Group (ECOG) Performance Status of 0 or 1.

Subjects were excluded from the study as a result of any concurrent chemotherapy, investigational product, biologic, or hormonal therapy for cancer treatment (concurrent use of hormonal therapy for non-cancer-related conditions (e.g., hormone replacement therapy) was acceptable. Subjects were also excluded from the study for radiotherapy treatment to more than 30% of the bone marrow or with a wide field of radiation within 4 weeks prior to the scheduled first dose of study treatment. Subjects were also excluded for prior receipt of any immune-mediated therapy or anti-angiogeneics. Subjects were also excluded from the study for use of immunosuppressive medications within 14 days before the first dose of study drug other than (i) intranasal, inhaled, or topical steroids, or local steroid injections and (ii) steroids as pre-medication for hypersensitivity reactions.

A minimum of 6 subjects were enrolled in Part 1, and up to 50 subjects per treatment arm were enrolled in Part 2.

### 2. Treatment

Following a screening period of up to 28 days, subjects were assigned (Part 1) or randomized (Part 2) to a study arm. In both study parts, treatment was administered until disease progression or any discontinuation criteria (e.g., withdraw of consent, unacceptable toxicity, noncompliance, confirmed progressive disease, etc.) were met.

Part 1 did not involve dose escalation. An initial group of 3 subjects was enrolled into Part 1 arms and evaluated for safety. Decisions in Part 1 were based on rules adapted from the modified toxicity probability interval-2 (mTPI-2) algorithm (Guo et al., Contemp Clin Trials 58:23-33 (2017)), which employed a simple beta-binomial Bayesian model. If the decision rule was to “stay” for the first 3 subjects, then an additional group of 2-4 subjects was enrolled at the same dose level; if the decision rule was to “de-escalate,” an additional group of 3 subjects were enrolled to a lower dose of oleclumab while maintaining the standard dose of FOLFOX plus bevacizumab plus durvalumab. If the decision rule was “completion,” the current dose was selected for Part 2 of the study.

In Part 2, randomization was evenly distributed across all arms (1:1:1) initially and was stratified based on location of the primary tumor (right sided vs. left sided). After 50 subjects were randomized to the control arm, the control arm continued to enroll subjects, but the allocation ratio to the different arms could be adjusted.

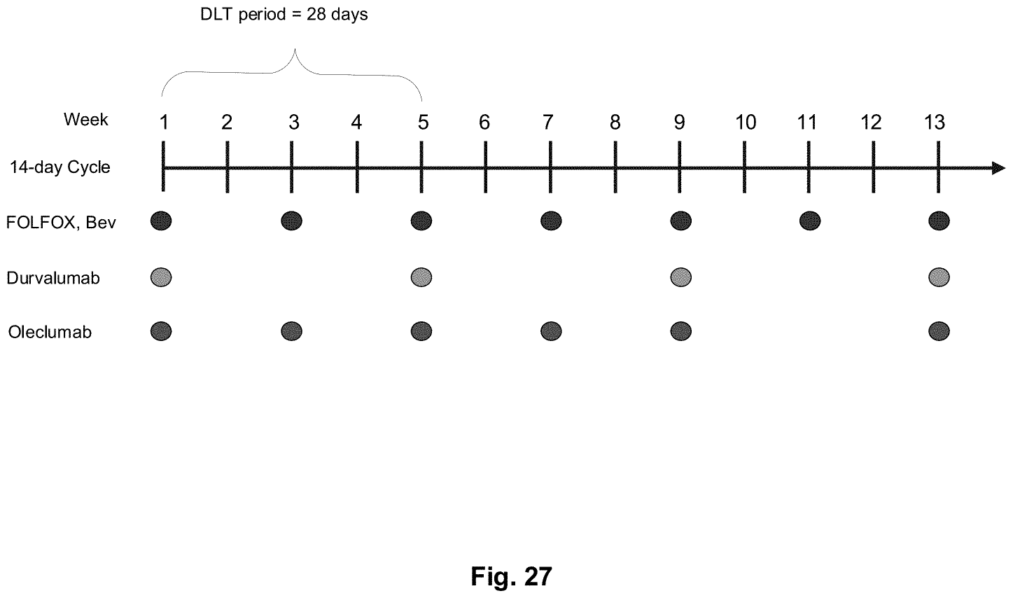

The treatment groups are provided in Table 5, and the treatment schedules s are provided in FIG. 27.

TABLE 5
Oleclumab + Durvalumab ± Chemotherapy Treatment Groups

| Study Part | Arm | Treatment |
|---|---|---|
| 1 | S1 | FOLFOX + bevacizumab + |
| | | durvalumab + oleclumab |
| 2 | Control 1 | FOLFOX + bevacizumab |
| 2 | E1 | FOLFOX + bevacizumab + |
| | | durvalumab + oleclumab |

Folinic acid (leucovorin)+5-fluorouracil+oxaliplatin (FOLFOX) plus bevacizumab were administered as outlined in the protocol per National Comprehensive Cancer Network (NCCN) and European Society for Medical Oncology (ESMO) guidelines. In particular, 400 mg/m² of folinic acid was administered intravenously every 2 weeks (Q2W) (Day 1 of every 14-day cycle); 85 mg/m² of oxaliplatin was administered by intravenous infusion Q2W (Day 1 of every 14-day cycle); and 2400 mg/m² of 5-fluorouracil was administered by continuous intravenous infusion over 46 to 48 hours Q2W (Day 1-2 of every 14-day cycle). 5-Fluorouracil was administered as infusion only, with no bolus. In addition, 5 mg/kg of bevacizumab was administered by intravenous infusion Q2W (Day 1 of every 14-day cycle).

In Arms S1 and E1, 1500 mg durvalumab was administered intravenously every 4 weeks (Q4W), and 3000 mg oleclumab was administered intravenously every 2 weeks (Q2W) for four doses and then Q4W starting on Cycle 5, Day 1.

### 3. Results

The objective response per RECIST v 1.1 is evaluated to demonstrate that the combination of oleclumab and durvalumab with FOLFOX plus bevacizumab has superior antitumor activity to FOLFOX with bevacizumab in subjects with 1 L MSS-CRC. Best overall response (BOR), duration of response (DoR), disease control (DC), 12-month progression free survival (PFS-12), and progression free survival (PFS) as assessed by RECIST v 1.1 and overall survival (OS) are also evaluated to demonstrate that the combination of oleclumab and durvalumab with FOLFOX plus bevacizumab has superior antitumor activity to FOLFOX with bevacizumab in subjects with 1 L MSS-CRC. Adverse events, dose-limiting toxicities (DLTs), laboratory findings, and vital signs are assessed to demonstrate that the combination of oleclumab and durvalumab with FOLFOX plus bevacizumab is safe.

### Example 10: Oleclumab and Durvalumab in Combination with Adjuvant Chemotherapy Treatment in Subjects with High-Risk Metastatic Microsatellite-Stable Colorectal Cancer

A Phase 2 Open-Label, Multicenter study was performed to assess the efficacy (antitumor activity) and safety of oleclumab and durvalumab in combination with adjuvant chemotherapy in subjects with high risk metastatic microsatellite-stable colorectal cancer (MSS-CRC).

### 1. Subjects

Subjects in this study included adult subjects at least 18 years of age (with a body weight of at least 35 kg) who had undergone radical surgical resection for Stage II or III MSS-CRC, were eligible for 6 months of mFOLFOX6 adjuvant therapy within 8 weeks after surgery, and were confirmed as having circulating tumor DNA (ctDNA) positive post-surgery. All subjects were required to be high risk Stage II: any T4 lesion or a T3 lesion with any one of the following characteristics: high grade (3), clinical presentation with bowel obstruction and perforation, histological signs of vascular, lymphatic and perineural invasion, ≤12 lymph nodes examined. Subjects must not have received prior systemic chemotherapy, immunotherapy, or radiotherapy for treatment of colorectal cancer (CRC) and must not have defective DNA mismatch repair (MSI). Subjects had a margin-negative (RO; defined as >1 mm clearance) surgical resection. All subjects had an Eastern Cooperative Oncology Group (ECOG) Performance Status of 0 or 1.

Subjects were excluded from the study if there was evidence of metastatic disease (including the presence of tumor cells in ascites or peritoneal carcinomatosis resected “en bloc”). Subjects were also excluded for concurrent chemotherapy, investigational product, biologic, or hormonal therapy for cancer treatment. Subjects were also excluded from the study for use of immunosuppressive medications within 14 days before the first dose of study drug other than (i) intranasal, inhaled, or topical steroids, or local steroid injections and (ii) steroids as pre-medication for hypersensitivity reactions.

### 2. Treatment

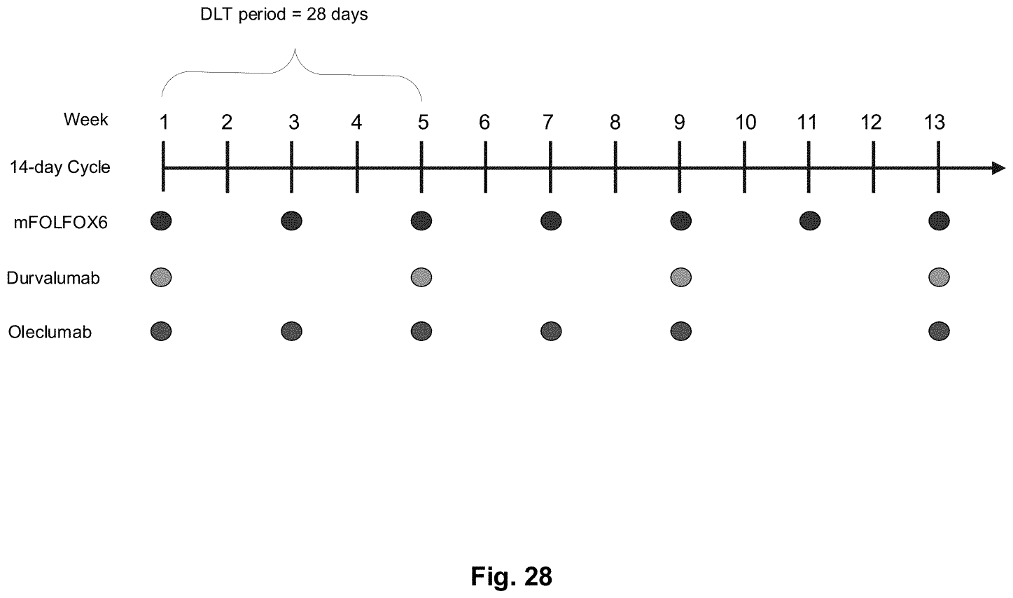

Subjects were randomized to one of the study arms. Approximately 40 subjects per treatment arm were enrolled. Randomization was strategized by the American Joint Committee on Cancer stage of the primary tumor (Stage II vs Stage III). The study arms and treatments are summarized in Table 6, and the dosing regimens are provided in FIG. 28.

TABLE 6
Oleclumab + Durvalumab ± Chemotherapy Treatment Groups

| Arm | Treatment |
|---|---|
| Control | mFOLFOX6 |
| E1-COC | mFOLFOX6 + Durvalumab |
| E2 | mFOLFOX6 + Durvalumab + oleclumab |

COC = contribution of components;
E = experimental;
mFOLFOX6 = folinic acid (leucovorin), 5-fluorouracil, oxaliplatin

In all arms, mFOLFOX6 was administered every 2 weeks (Q2W) as outlined in the protocol per National Comprehensive Cancer Network and European Society for Medical Oncology guidelines. In particular, oxaliplatin was administered at a dose of 85 mg/m² by intravenous (IV) infusion (Day 1 of every 14-day cycle) limited to a maximum body surface area (BSA) of 2.0 m². Folinic acid (leucovorin) was administered at a dose of 400 mg/m² by IV infusion (Day 1 of every 14-day cycle). Fluorouracil (5-FU) was administered at a dose of 400 mg/m² by IV bolus on Day 1, then 1,200 mg/m²/day for 2 days (total 2,400 mg/m² over 46-48 hours) IV infusion (Days 1-2 of every 14-day cycle). Durvalumab was administered at a dose of 1500 mg IV every 4 weeks (Q4W), and oleclumab was administered at a dose of 3000 mg IV Q2W for four doses then Q4W starting at Cycle 5.

Subjects were treated for up to 6 months or until recurrence, unacceptable toxicity, withdrawal of consent, etc. Subjects could be followed for up to 5 years from randomization.

### 3. Results

The clearance of circulating tumor DNA (ctDNA) at 6 months is evaluated to demonstrate that the combination of oleclumab and durvalumab with mFOLFOX6 has superior antitumor activity to mFOLFOX6 in subjects with high risk Stage II or Stage III MSS-CRC. CtDNA clearance is defined as the ctDNA status change from ctDNA positive at baseline to ctDNA negative post-randomization, and comparison between groups is performed using the Cochran-Matnel-Haenszel test stratified by the disease stage at a significance level of 0.2 (2-sided). Disease free survival (DFS), DFS at 12 months (DFS-12), and overall survival (OS) are also compared to demonstrate that the combination of oleclumab and durvalumab with mFOLFOX6 has superior antitumor activity to FOLFOX in subjects with high risk Stage II or Stage III MSS-CRC

Adverse events, laboratory findings, and vital signs are assessed to demonstrate that the combination of oleclumab and durvalumab with mFOLFOX6 is safe.

### Example 11: Anti-CD73 and Anti-PD-L1 in Combination with Chemotherapy in Colorectal and Fibrosarcoma Models

Assays were performed in murine colorectal and fibrosarcoma models to demonstrate the efficacy of anti-CD73 and anti-PD-L1 antibodies in combination with chemotherapy.

### 1. Materials and Methods

### Animals

The in vivo studies were performed using 8 weeks old BALB/cAnNCtr mice (Charles River UK) or C57BL/6. The animals were housed in an AstraZeneca vivarium with access to food and water ad libidum and were cared for daily by trained personnel. Mice were handled according to the Home Office Animals Scientific Procedures Act, 1986, UK.

### In Vivo Efficacy Assays

The animals were implanted subcutaneously with murine syngeneic tumour lines, either 0.5e6 CT26 (mouse colorectal) or 0.5e6 MCA205 (50% Matrigel) (mouse fibrosarcoma) depending on mouse strain. Tumour progression was monitored by caliper measurements 3 times a week. The animals were treated (as monotherapy or in different combinations) with anti-CD73 mouse IgG1 (in house, AstraZeneca) starting on day 3, 10 mg/kg, twice weekly, 4 doses; anti-PD-L1 mouse IgG1 D265A (in house, AstraZeneca) starting on day 10 (when combined with OHP and 5FU) or 4 (when combined with Docetaxel), 10 mg/kg, twice weekly up-to a total of 6 doses (see figure legends for information concerning specific experiments); 5-fluorouracil (Fresenius Kabi) and oxaliplatin (Accord) on day 9 (or day 10 in some experiments), single dose, 50 mg/kg and 6 mg/kg; Docetaxel (Sanofi), starting on day 4, 10 mg/kg, once weekly, 2 doses, respectively. The two antibodies, plus 5FU and OHP were administered intraperitoneally, while Docetaxel was administered intravenously. The animals were humanely sacrificed once the tumor dimensions reached 15 mm in diameter.

### In Vivo Pharmacodynamic Assays

The animals were implanted with 0.5e6 CT26 mouse colorectal carcinoma cells subcutaneously. Tumour progression was monitored by caliper measurements 3 times a week. The animals were treated (as monotherapy or in different combinations) with anti-CD73 mouse IgG1 (in house, AstraZeneca) starting on day 3, 10 mg/kg, twice weekly, 4 doses; anti-PD-L1 mouse IgG1 D265A (in house, AstraZeneca) starting on day 10, 10 mg/kg, twice weekly, 2 doses; 5-fluorouracil (Fresenius Kabi) and oxaliplatin (Accord), on day 10, single dose, 50 mg/kg and 6 mg/kg, respectively. All drugs were administered intraperitoneally. The animals were humanely sacrificed on day 15 post implantation and the tumours were used in downstream analyses.

### Tissue Processing and Flow Cytometry

The tumours were digested using an enzyme cocktail of 1 mg/mL collagenase IV, 20 units/mL DNase I and 20 units/mL hyaluronidase I (all from Sigma). The single cell suspension was then stained with a live/dead differentiating dye (see table 7 below) and treated with Fc block (anti-mouse CD16/CD32 eBioScience cat #14-0161-86). Subsequently, the cells were stained for surface markers (for reagent list see table & below) and fixed and permeabilised using the eBioScience Foxp3/transcription factor staining kit (00-5523-00). Next, the cells were stained for intracellular markers (see table 7 below). The samples were acquired on a BD Symphony flow cytometer and analysed using FlowJo software version 10. The data was plotted using the GraphPad Prizm software.

TABLE 7
Reagents for tissue processing and flow cytometry

| Reagent | Fluorophore | Supplier | Cat number |
|---|---|---|---|
| Zombie UV Fixable | n/a | eBioScience | 65-0866-14 |
| Viability kit eF506 | | | |
| Brilliant stain buffer | n/a | Becton Dickinson | 566349 |
| True-stain monocyte | n/a | BioLegend | 426103 |
| blocker | | | |
| Anti-mouse CD45 | BV785 | BioLegend | 103149 |
| Anti-mouse CD3 | BUV395 | Becton Dickinson | 563565 |
| Anti-mouse CD4 | BUV661 | Becton Dickinson | 612974 |
| Anti-mouse CD8 | FITC | BioLegend | 100706 |
| Anti-mouse NKp46 | BV605 | BioLegend | 137619 |
| Anti-mouse CD25 | PerCPCy5.5 | BioLegend | 102007 |
| Anti-mouse Ki67 | APC | eBioScience | 51-5698-82 |
| Anti-mouse IFNg | BV711 | BioLegend | 505836 |
| Anti-mouse Foxp3 | eF450 | eBioScience | 48-5773-82 |
| Anti-mouse CD73* | PE | In house | n/a |
| Anti-mouse CD38 | AF700 | Thermo Fisher | 56-0381-82 |
| | | Scientific | |
| Anti-mouse CD39 | PE-Cy7 | BioLegend | 143806 |
| Anti-mouse PD-1 | APCeF780 | Thermo Fisher | 47-9985-82 |
| | | Scientific | |

*The anti-CD73 antibody used in this panel was produced and conjugated in house using PE/R-Phycoerythrin Conjugation Kit - Lightning-Link ® (ab102918).

### 2. Results

### Anti-CD73+Anti-PD-L1+5FU+OHP in CT26 (Colorectal) Model

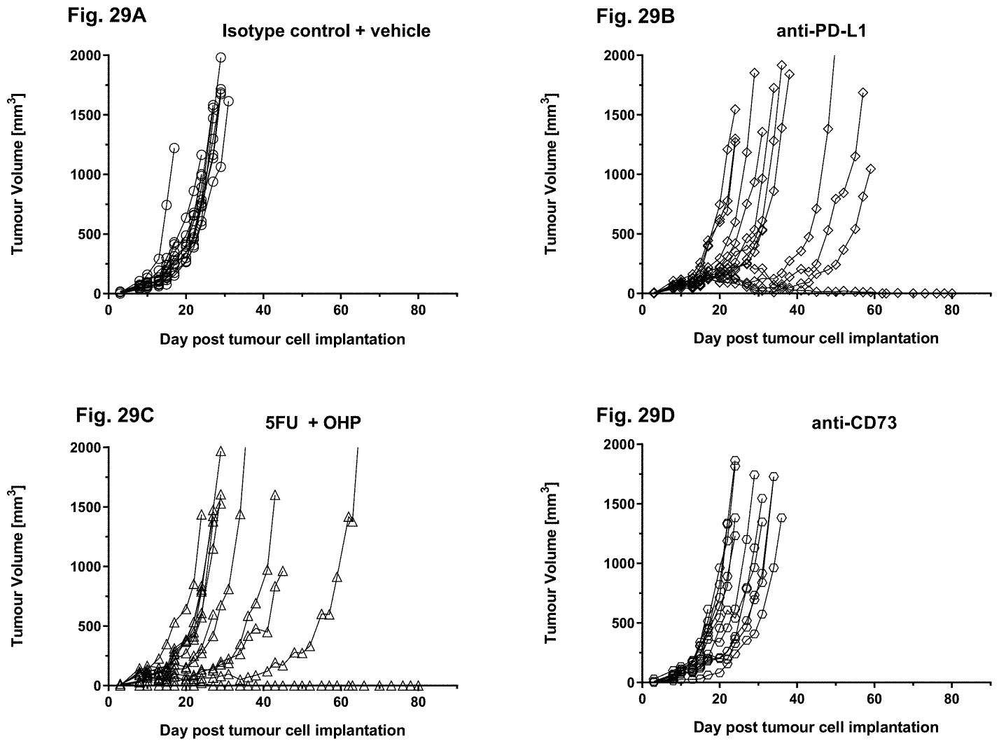

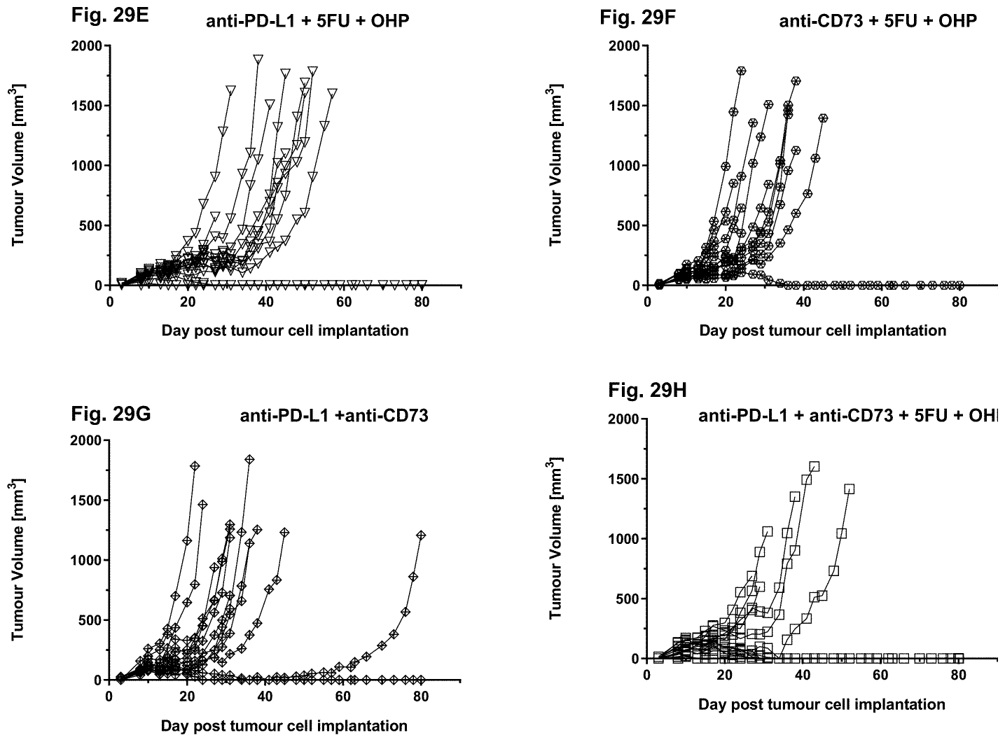

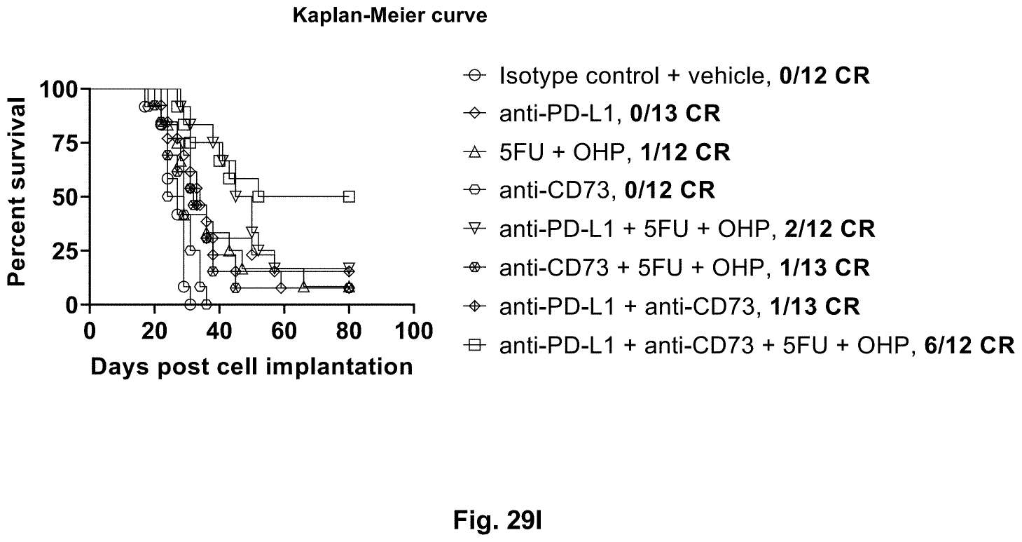

In order to analyse efficacy of anti-CD73 and anti-PD-L1 antibodies in combination with 5FU and OHP in the CT26 model, animals were implanted with 0.5e6 CT26—subcutaneously (s.c.) and treated with anti-mouse antibodies intraperitoneally (i.p.) twice weekly (anti-CD73 starting on day 3 post implantation, 4 doses, and anti-PD-L1 on day 10, 6 doses). 5 fluorouracil (5FU) and oxaliplatin (OHP) were administered i.p. on day 9. The animals were sacrificed humanely once the tumour diameter approached 15 mm. Animals that were sacrificed early due to welfare issues such as tumour condition were excluded from the analysis. For survival calculations, animals sacrificed due to tumour condition before the tumour diameter was 15 mm were left in the analyses if the tumour volume was over 500 mm³. The results are shown in FIGS. 29A-I.

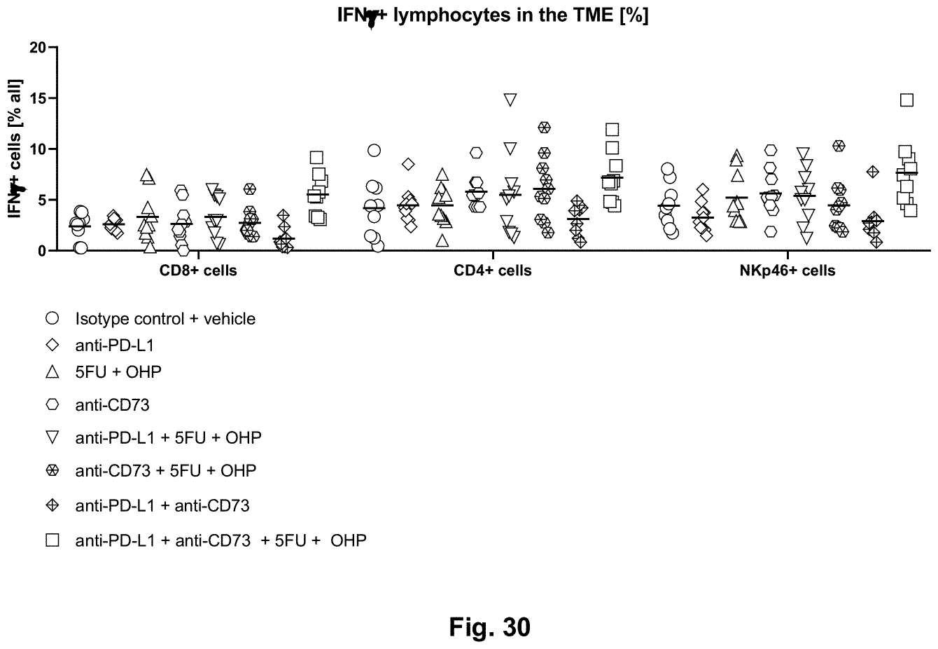

In further analysis of activity in the CT26 model, animals were implanted with 0.5e6 CT26 s.c. and treated with anti-mouse antibodies i.p. twice weekly (anti-CD73 starting on day 3 post implantation, 4 doses, and anti-PD-L1 on day 10, 2 doses). 5FU and OHP were administered i.p. on day 9. FIG. 30 presents data from whole tumor digests (samples collected on day 15 post implantation) analysed by flow cytometry without ex vivo re-stimulation (intracellular staining).

### Anti-CD73+Anti-PD-L1+Docetaxel in CT26 (Colorectal) Model

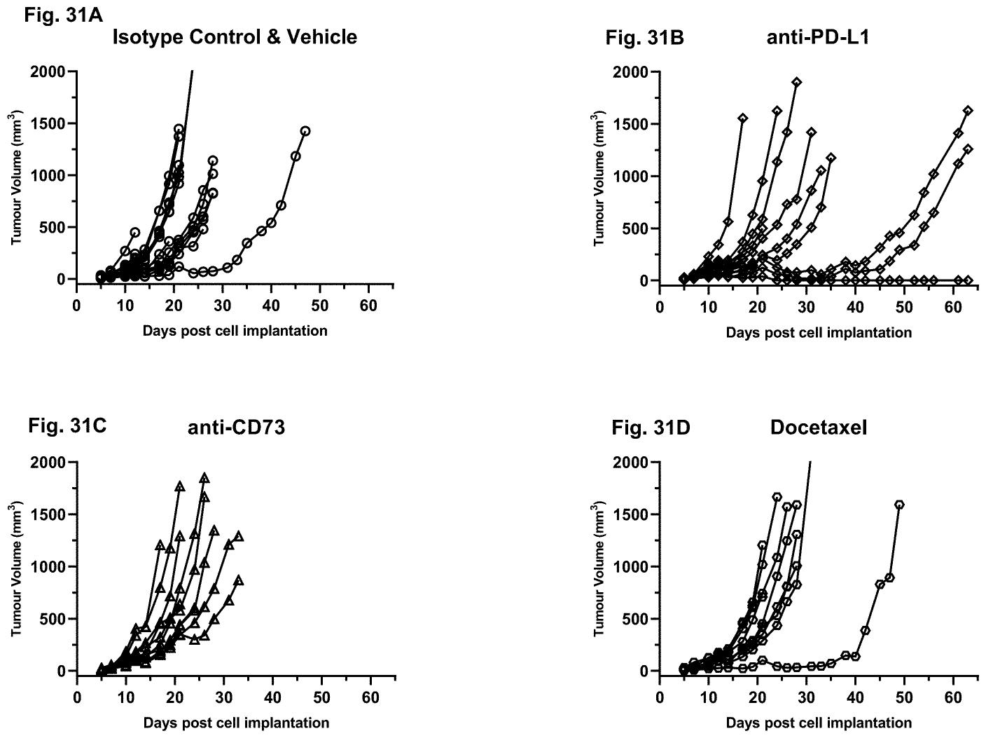

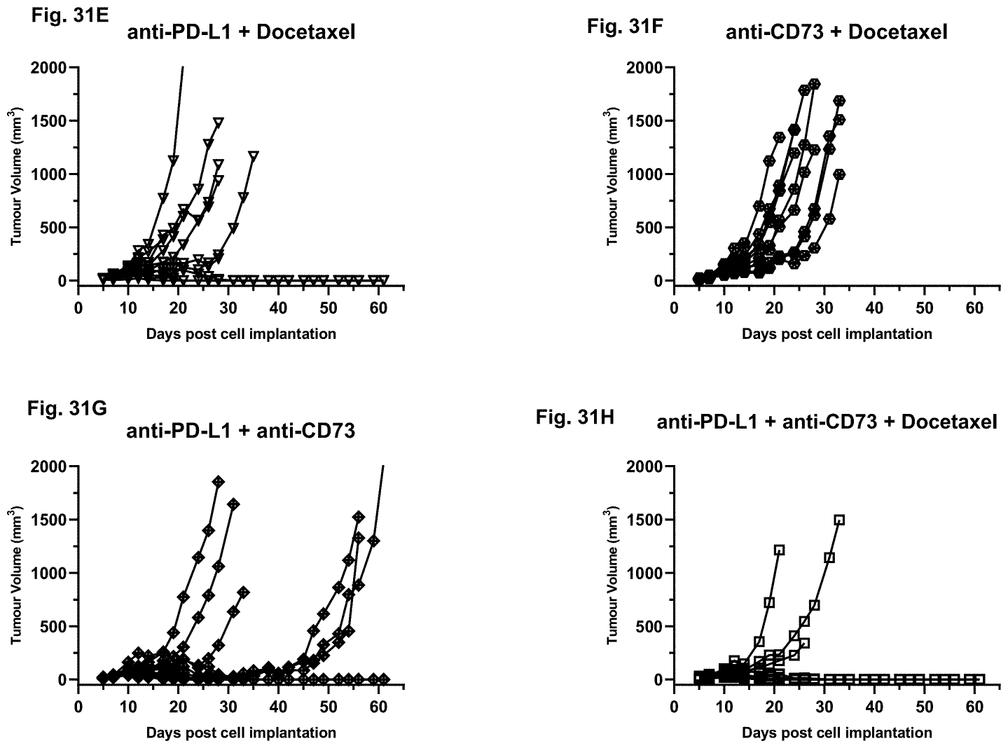

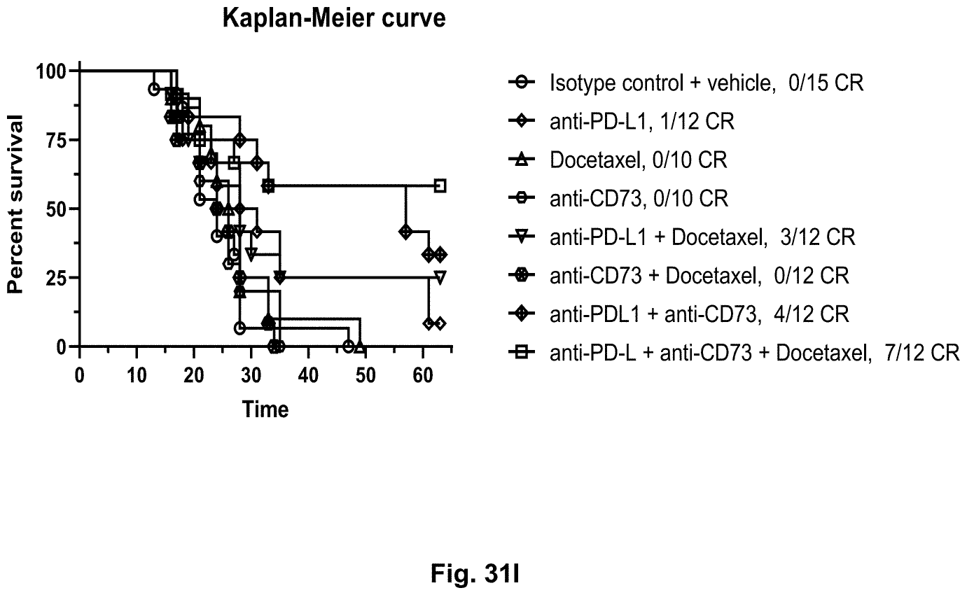

In order to analyse efficacy of anti-CD73 and anti-PD-L1 antibodies in combination with docetaxel in the CT26 model, animals were implanted with 0.5e6 CT26 s.c. and treated with anti-mouse antibodies i.p. twice weekly (anti-CD73 starting on day 3 post implantation, 4 doses, and anti-PD-L1 on day 4, 4 doses). Docetaxel was administered i.v., once weekly (starting day 4, 2 doses). The animals were sacrificed humanely once the tumour diameter approached 15 mm. Animals which were sacrificed early due to welfare issues such as tumour condition were excluded from the analysis. For survival calculations, animals sacrificed due to tumour condition before the tumour diameter was 15 mm were left in the analyses if the tumour volume was over 500 mm³. The results are shown in FIGS. 31A-I.

### Anti-CD73+Anti-PD-L1+5FU+OHP in MCA205 (Fibrosarcoma) Model

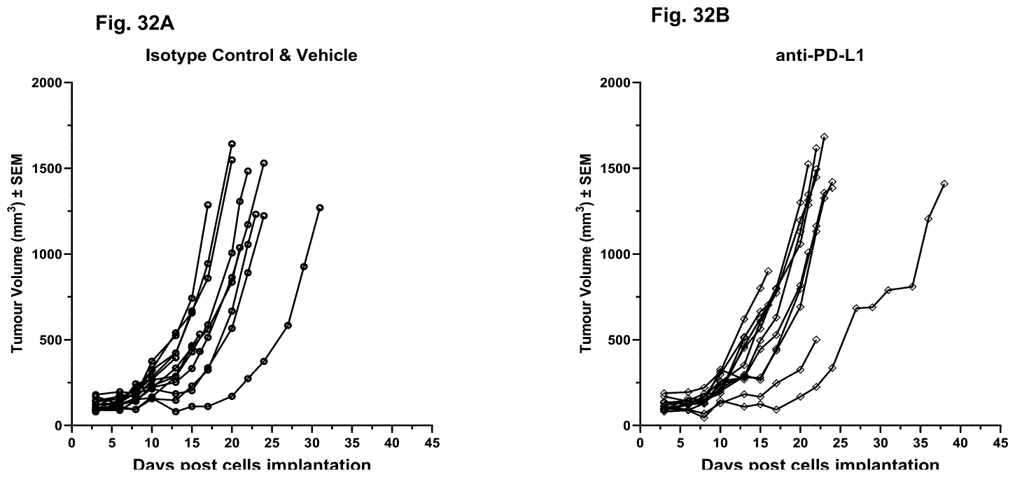

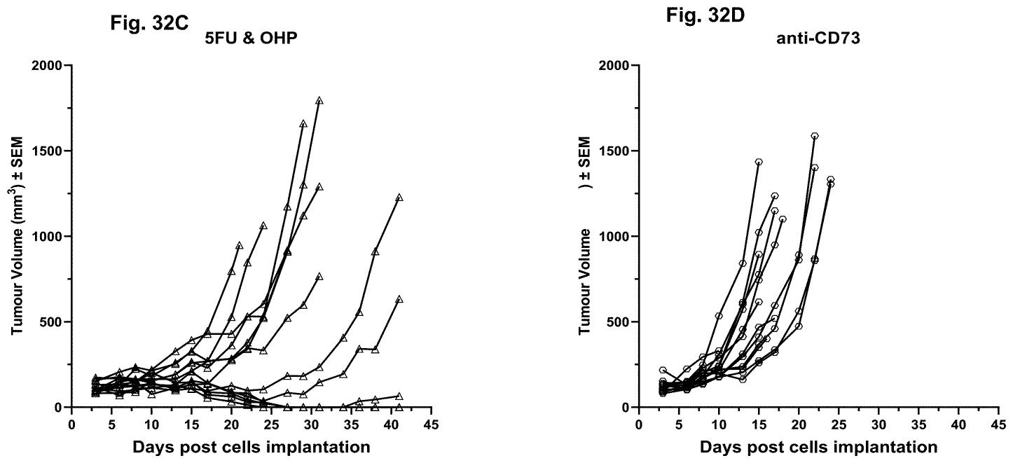

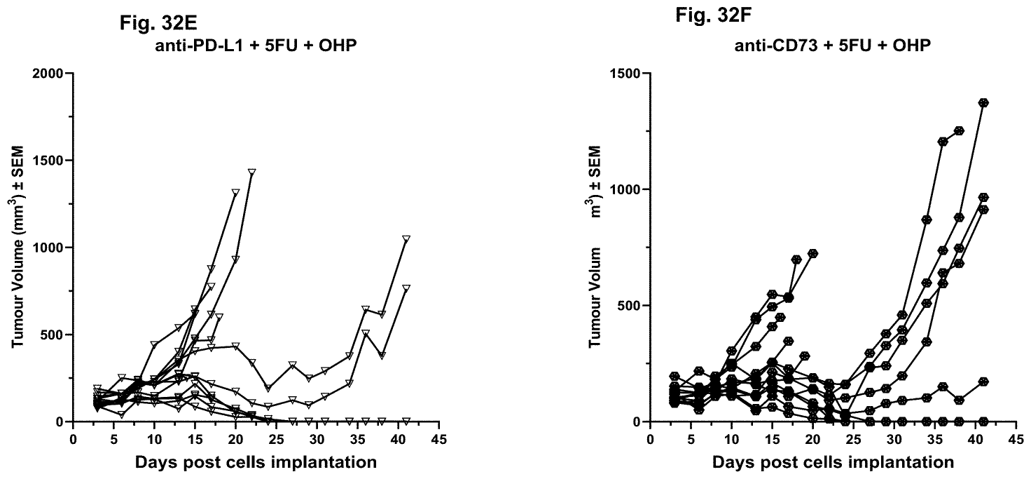

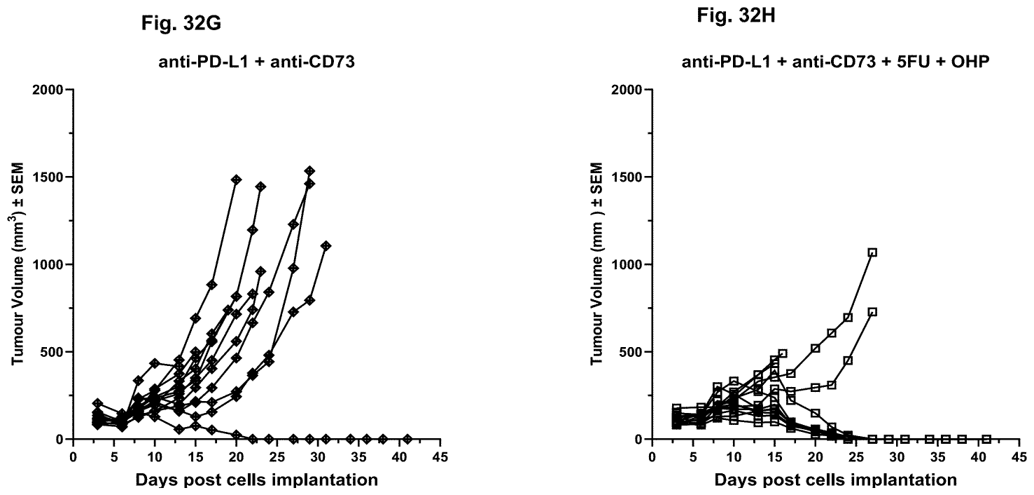

In order to analyse efficacy in the MCA205 model, animals were implanted with 0.5e6 MCA205 (50% Matrigel) s.c. and treated with anti-mouse antibodies i.p. twice weekly (anti-CD73 starting on day 3 post implantation, 4 doses, and anti-PD-L1 on day 10, 5 doses). 5FU and OHP were administered i.p. on day 9. The results are shown in FIGS. 32A-H.

### 3. Conclusions

In mice implanted with CT26 tumours, the combination of anti-PD-L1+anti-CD73+5FU+OHP resulted in 6 out of 12 (50%) complete responses compared to maximum of 2 out of 12 (˜17%) in the anti-PD-L1+5FU+OHP combination group. In addition, the combination of anti-PD-L1+anti-CD73+docetaxel resulted in 7 out of 12 (58%) complete responses compared to maximum of 3 out of 12 (25%) in the anti-PD-L1+docetaxel combination group.

The percentage of IFNγ+CD8+, CD4+ and NKp46+lymphocytes increased in the tumour microenvironment (TME) in samples from animals treated with a combination of anti-CD73, anti-PD-L1, 5FU and OHP.

In mice implanted with MCA205 tumours, the combination of anti-PD-L1+anti-CD73+5FU+OHP results in 8 out of 13 (61%) complete responses compared to 4 out of 13 (30%) in the anti-PD-L1+5FU+OHP combination group.

These data indicate that anti-CD73 antibodies increase the efficacy of anti-PD-L1 plus chemotherapy (including e.g., 5FU+OHP and docetaxel) treatments, in multiple cancer types.

While the invention has been described in terms of various embodiments, it is understood that variations and modifications will occur to those skilled in the art. Therefore, it is intended that the appended claims cover all such equivalent variations that come within the scope of the invention as claimed. In addition, the section headings used herein are for organizational purposes only and are not to be construed as limiting the subject matter described.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

All references cited in this application are expressly incorporated by reference herein.

TABLE 8
Disclosed Sequences

| SEQ | | |
|---|---|---|
| ID | | |
| NO: | Sequence | Description |
| 1 | QSVLTQPPSASGTPGQRVTISCSGSLSNIGRN | Light chain |
| | PVNWYQQLPGTAPKLLIYLDNLRLSGVPDRFS | variable |
| | GSKSGTSASLAISGLQSEDEADYYCATWDDSH | domain of |
| | PGWTFGGGTKLTVL | oleclumab |
| 2 | EVQLLESGGGLVQPGGSLRLSCAASGFTFSSY | Heavy chain |
| | AYSWVRQAPGKGLEWVSAISGSGGRTYYADSV | variable |
| | KGRFTISRDNSKNTLYLQMNSLRAEDTAVYYC | domain of |
| | ARLGYGRVDEWGRGTLVTVSS | oleclumab |
| 3 | SYAYS | CDRH1 of |
| | | oleclumab |
| 4 | AISGSGGRTYYADSVKG | CDRH2 of |
| | | oleclumab |
| 5 | LGYGRVDE | CDRH3 of |
| | | oleclumab |
| 6 | SGSLSNIGRNPVN | CDRL1 of |
| | | oleclumab |
| 7 | LDNLRLS | CDRL2 of |
| | | oleclumab |
| 8 | ATWDDSHPGWT | CDRL3 of |
| | | oleclumab |
| 9 | EIVLTQSPGTLSLSPGERATLSCRASQRVSSS | Light chain |
| | YLAWYQQKPGQAPRLLIYDASSRATGIPDRFS | variable |
| | GSGSGTDFTLTISRLEPEDFAVYYCQQYGSLP | domain of |
| | WTFGQGTKVEIK | durvalumab |
| 10 | EVQLVESGGGLVQPGGSLRLSCAASGFTFSRY | Heavy chain |
| | WMSWVRQAPGKGLEWVANIKQDGSEKYYVDSV | variable |
| | KGRFTISRDNAKNSLYLQMNSLRAEDTAVYYC | domain of |
| | AREGGWFGELAFDYWGQGTLVTVSS | durvalumab |
| 11 | GFTFSRYWMS | CDRH1 of |
| | | durvalumab |
| 12 | NIKQDGSEKYYVDSVKG | CDRH2 of |
| | | durvalumab |
| 13 | EGGWFGELAFDY | CDRH3 of |
| | | durvalumab |
| 14 | RASQRVSSSYLA | CDRL1 of |
| | | durvalumab |
| 15 | DASSRAT | CDRL2 of |
| | | durvalumab |
| 16 | QQYGSLPWT | CDRL3 of |
| | | durvalumab |
| 17 | DIQMTQSPSSLSASVGDRVTITCSASQDISNY | Light chain |
| | LNWYQQKPGKAPKVLIYFTSSLHSGVPSRFSG | variable |
| | SGSGTDFTLTISSLQPEDFATYYCQQYSTVPW | domain of |
| | TFGQGTKVEIKRTVAAPSVFIFPPSDEQLKSG | bevacizumab |
| | TASVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |
| 18 | EVQLVESGGGLVQPGGSLRLSCAASGYTFTNY | Heavy chain |
| | GMNWVRQAPGKGLEWVGWINTYTGEPTYAADF | variable |
| | KRRFTFSLDTSKSTAYLQMNSLRAEDTAVYYC | domain of |
| | AKYPHYYGSSHWYFDVWGQGTLVTVSSASTKG | bevacizumab |
| | PSVFPLAPSSKSTSGGTAALGCLVKDYFPEPV | |
| | TVSWNSGALTSGVHTFPAVLQSSGLYSLSSVV | |
| | TVPSSSLGTQTYICNVNHKPSNTKVDKKVEPK | |
| | SCDKTHTCPPCPAPELLGGPSVFLFPPKPKDT | |
| | LMISRTPEVTCVVVDVSHEDPEVKFNWYVDGV | |
| | EVHNAKTKPREEQYNSTYRVVSVLTVLHQDWL | |
| | NGKEYKCKVSNKALPAPIEKTISKAKGQPREP | |
| | QVYTLPPSREEMTKNQVSLTCLVKGFYPSDIA | |
| | VEWESNGQPENNYKTTPPVLDSDGSFFLYSKL | |
| | TVDKSRWQQGNVFSCSVMHEALHNHYTQKSLS | |
| | LSPGK | |
| 19 | GYTFTNYGMN | CDRH1 of |
| | | bevacizumab |
| 20 | WINTYTGEPTYAADFKR | CDRH2 of |
| | | bevacizumab |
| 21 | YPHYYGSSHWYFDV | CDRH3 of |
| | | bevacizumab |
| 22 | SASQDISNYLN | CDRL1 of |
| | | bevacizumab |
| 23 | FTSSLHS | CDRL2 of |
| | | bevacizumab |
| 24 | QQYSTVPWT | CDRL3 of |
| | | bevacizumab |

What is claimed is:

1. A method of treating a tumor in a human patient, comprising administering oleclumab or antigen-binding fragment thereof to the patient.

2. A method of treating a tumor in a human patient, comprising administering oleclumab or antigen-binding fragment thereof and durvalumab or antigen-binding fragment thereof to the patient.

3. A method of treating a tumor in a human patient, comprising administering oleclumab or antigen-binding fragment thereof and chemotherapy to the patient.

4. The method of claim 3, further comprising administering durvalumab or antigen-binding fragment thereof.

5. The method of any one of claims 1-4, wherein the oleclumab or antigen-binding fragment thereof is administered at a dose of 750 mg to 3000 mg.

6. The method of claim 5, wherein the oleclumab or antigen-binding fragment thereof is administered at a dose of 750 mg.

7. The method of claim 5, wherein the oleclumab or antigen-binding fragment thereof is administered at a dose of 1500 mg.

8. The method of claim 5, wherein the oleclumab or antigen-binding fragment thereof is administered at a dose of 2250 mg.

9. The method of claim 5, wherein the oleclumab or antigen-binding fragment thereof is administered at a dose of 3000 mg.

10. The method of claim 5, wherein the oleclumab or antigen-binding fragment thereof is administered at a dose of 2250 mg and then at a dose of 3000 mg.

11. The method of claim 10, wherein the oleclumab or antigen-binding fragment thereof is administered at a dose of 2250 mg for four doses and then at a dose of 3000 mg.

12. The method of any one of claims 1-11, wherein oleclumab or antigen-binding fragment thereof is administered every 14 to 28 days.

13. The method of any one of claims 1-11, wherein oleclumab or antigen-binding fragment thereof is administered every 14 days.

14. The method of any one of claims 1-11, wherein oleclumab or antigen-binding fragment thereof is administered every 28 days.

15. The method of any one of claims 1-11, wherein the oleclumab or antigen-binding fragment thereof is administered every 14 days for at least two doses and then every 28 days.

16. The method of any one of claims 1-11, wherein the oleclumab or antigen-binding fragment thereof is administered every 14 days for four doses and then every 28 days.

17. The method of any one of claims 1-11, wherein the oleclumab or antigen-binding fragment thereof is administered every 21 days.

18. The method of any one of claims 1-11, wherein the oleclumab or antigen-binding fragment thereof is administered every 21 days for at least two doses and then every 28 days.

19. The method of any one of claims 1-11, wherein the oleclumab or antigen-binding fragment thereof is administered every 21 days for two to four doses and then every 28 days.

20. The method of claim 13, wherein the oleclumab or antigen-binding fragment thereof is administered every 21 days for two doses and then once every 28 days.

21. The method of claim 13, wherein the oleclumab or antigen-binding fragment thereof is administered once every 21 days for four doses and then once every 28 days.

22. The method of any one of claims 1-5, wherein the oleclumab or antigen-binding fragment thereof is administered at a dose of 2250 mg once every 21 days for two doses and then at a dose of 3000 mg once every 28 days.

23. The method of any one of claims 1-5, wherein the oleclumab or antigen-binding fragment thereof is administered at a dose of 2250 mg once every 21 days for four doses and then at a dose of 3000 mg once every 28 days.

24. The method of any one of claims 1-23, wherein the oleclumab or antigen-binding fragment thereof is administered intravenously.

25. The method of any one of claims 2 and 4-24, wherein the durvalumab or antigen-binding fragment thereof is administered at a dose of 1500 mg.

26. The method of any one of 2 and 4-25, wherein the durvalumab or antigen-binding fragment thereof is administered every 21 days to every 28 days.

27. The method of any one of claims 2 and 4-26, wherein the durvalumab or antigen-binding fragment thereof is administered every 28 days.

28. The method of any one of claims 2 and 4-26, wherein the durvalumab or antigen-binding fragment thereof is administered every 21 days.

29. The method of any one of claims 2 and 4-26, wherein the durvalumab or antigen-binding fragment thereof is administered every 21 days for at least two doses and then every 28 days.

30. The method of any one of claims 2 and 4-26, wherein the durvalumab or antigen-binding fragment thereof is administered every 21 days for four doses and then every 28 days.

31. The method of any one of claims 2 and 4-26, wherein the durvalumab or antigen-binding fragment thereof is administered at a dose of 1500 mg every 21 days for four doses and then at a dose of 1500 mg every 28 days.

32. The method of any one of claims 1-31, wherein the durvalumab or antigen-binding fragment thereof is administered intravenously.

33. The method of any one of claims 3-32, wherein the chemotherapy comprises at least one of cisplatin, pemetrexed. nab-paclitaxel, carboplatin, gemcitabine, cisplatin, oxaliplatin, leucovorin, 5-fluorouracil, and docetaxel.

34. The method of claim 33, wherein the chemotherapy comprises oxaliplatin, leucovorin, and 5-fluorouracil.

35. The method of claim 33 or 34, wherein the oxaliplatin is administered at a dose of 85 mg/m².

36. The method of any one of claims 33-35, wherein the oxaliplatin is administered every 2 weeks.

37. The method of any one of claims 33-36, wherein the leucovorin is administered at a dose of 400 mg/m².

38. The method of any one of claims 33-38, wherein the leucovorin is administered every 2 weeks.

39. The method of any one of claims 33-38, wherein the 5-fluorouracil is administered at a dose of 2400 mg/m².

40. The method of any one of claims 33-39, wherein the 5-fluorouracil is administered by continuous intravenous infusion for 46 to 48 hours.

41. The method of any one of claims 33-40, wherein the 5-fluorouracil is administered over 46 to 48 hours every 2 weeks.

42. The method of any one of claims 33-41, wherein the chemotherapy comprises 85 mg/m² oxaliplatin, 400 mg/m² leucovorin and 2400 mg/m² 5-fluorouracil.

43. The method of any one of claims 34-42, further comprising administering bevacizumab or an antigen-binding fragment thereof.

44. The method of claim 43, wherein bevacizumab or an antigen-binding fragment thereof is administered at a dose of 5 mg/kg.

45. The method of claim 43 or 44, wherein bevacizumab or an antigen-binding fragment thereof is administered every 2 weeks.

46. The method of any one of claims 43-45, wherein bevacizumab or an antigen-binding fragment thereof is administered intravenously.

47. The method of claim 33, wherein the chemotherapy comprises (a) nab-paclitaxel and carboplatin; (b) gemcitabine and cisplatin; (c) gemcitabine and carboplatin; (d) pemetrexed and carboplatin; and (e) pemetrexed and cisplatin.

48. The method of claim 33 or 47, wherein the nab-paclitaxel is administered at a dose of 100 mg/m².

49. The method of any one of claim 33, 47, or 48, wherein the nab-paclitaxel is administered on days 1, 8, and 15 of a 21-day cycle.

50. The method of claim 33 or 47, wherein the gemcitabine is administered at a dose of 1000 mg/m² or 1250 mg/m².

51. The method of any one of claim 33, 47, or 50, wherein the gemcitabine is administered on days 1 and 8 of a 21-day cycle.

52. The method of claim 33 or 47 wherein the pemetrexed is administered at a dose of 500 mg/m².

53. The method of any one of claims 33, 47, and 52, wherein the pemetrexed is administered every three weeks.

54. The method of any one of claims 33, and 47-53, wherein the carboplatin is administered at a dose of AUC 5 or 6.

55. The method of any one of claims 33 and 47-55, wherein the carboplatin is administered every three weeks.

56. The method of any one of claims 33, 47, and 50-53, wherein the cisplatin is administered at a dose of 75 mg/m².

57. The method of any one of claims 33, 47, 50-53, and 56 wherein the cisplatin is administered every three weeks.

58. The method of claim 33, wherein the chemotherapy comprises 1000 mg/m² gemcitabine and 125 mg/m² nab-paclitaxel.

59. The method of any one of claims 3-58, wherein the chemotherapy is administered every 7 days to 28 days.

60. The method of claim 59, wherein the chemotherapy is administered every 14 days.

61. The method of any one of claims 1 and 5-24, wherein the administration of oleclumab or antigen-binding fragment thereof results in a partial response.

62. The method of any one of claims 1 and 5-24, wherein the administration of oleclumab or antigen-binding fragment thereof results in a complete response.

63. The method of any one of claims 1-62, wherein the tumor is a solid tumor.

64. The method of claim 63, wherein the solid tumor is breast cancer, ovarian cancer, head and neck cancer, prostate cancer, bladder cancer, colorectal cancer, non-small cell lung cancer (NSCLC), gliobastoma, renal cell cancer, or pancreatic cancer.

65. The method of claim 64, wherein the pancreatic cancer is pancreatic ductal adenocarcinoma.

66. The method of claim 64, wherein the tumor is a resectable NSCLC tumor.

67. The method of claim 64 or 66, wherein the tumor is an early-stage NSCLC tumor.

68. The method of claim 64, wherein the tumor is stage IV NSCLC tumor.

69. The method of claim 64, wherein the colorectal cancer is metastatic microsatellite-stable.

70. The method of any one of claims 1-64, wherein the tumor has high-PD-L1 expression, optionally wherein the tumor is a NSCLC tumor.

71. The method of any one of claims 1-64, wherein the tumor has low-PD-L1 expression, optionally wherein the tumor is a NSCLC tumor.

72. The method of any one of claims 1-64, wherein the tumor lacks an activating epidermal growth factor receptor (EGFR) mutation and/or an anaplastic lymphoma kinase (ALK) fusion, optionally wherein the tumor is a NSCLC tumor.

73. The method of any one of claims 1-64 wherein the patient has metastatic pancreatic ductal adenocarcinoma that has not been previously treated.

74. The method of any one of claims 1-64, wherein the patient has metastatic pancreatic ductal adenocarcinoma that was previously treated with gemcitabine-based therapy.

75. The method of any one of claims 1-74, wherein the tumor has not received prior treatment in the recurrent and/or metastatic setting.

76. The method of any one of claims 1-74, wherein the patient has progressed on an anti-PD-1 or anti-PD-L1 containing therapy.

77. The method of any one of claims 3, 24, and 32, wherein the tumor is a 1^{st} line metastatic pancreatic ductal adenocarcinoma, wherein the oleclumab or antigen binding fragment thereof is administered at 1500 mg or 3000 mg every 2 weeks for four doses and then every 4 weeks, and wherein the chemotherapy comprises 1000 mg/m² gemcitabine and 125 mg/m² nab-paclitaxel, wherein the chemotherapy is administered on days 1, 8, and 15 of four 28-day cycles and then every 4 weeks.

78. The method of any one of claims 3, 24, and 32, wherein the tumor is a 2^{nd} line metastatic pancreatic ductal adenocarcinoma, the oleclumab or antigen binding fragment thereof is administered at 1500 mg or 3000 mg every 2 weeks for four doses and then every 4 weeks, and wherein the chemotherapy comprises 85 mg/m² oxaliplatin, 400 mg/m² leucovorin, and 400 mg/m² 5-FU followed by 2400 mg/m² 5-FU, wherein the chemotherapy is administered on days 1 and 15 of four 28-day cycles and then every 4 weeks.

79. The method of claim 77 or 78, further comprising administering 1500 mg durvalumab or an antigen-binding fragment thereof every 4 weeks.

80. The method of any one of claims 2, 24, and 32, wherein the tumor is a 1^{st} line stage IV NSCLC with high PD-L1 expression, wherein the oleclumab or antigen binding fragment thereof is administered at 1500 mg or 3000 mg every 2 weeks for two 28-day cycles and then every 4 weeks, and wherein the durvalumab or an antigen-binding fragment thereof is administered at 1500 mg every 4 weeks.

81. The method of any one of claims 4, 24, and 32, wherein the tumor is a 1^{st} line stage IV NSCLC with low PD-L1 expression, wherein
(i) the oleclumab or antigen binding fragment thereof is administered (a) at 1500 mg every 3 weeks for four 21-day cycles and then every 4 weeks; or (b) at 2250 mg every 3 weeks for four 21-day cycles and then at 3000 mg every 4 weeks;
(ii) the durvalumab or antigen binding fragment thereof is administered at 1500 mg every 3 weeks for four 21-day cycles and then every 4 weeks; and
(iii) the chemotherapy comprises: (a) 100 mg/m² nab-paclitaxel on days 1, 8, and 15 of a 21-day cycle for 4 cycles and 5 or 6 AUC carboplatin on day 1 of the 21-day cycle for 4 cycles; (b) 1000 mg/m² or 1250 mg/m² gemcitabine on days 1 and 8 of a 21-day cycle for 4 cycles and 75 mg/m² cisplatin on day 1 of the 21-day cycle for 4 cycles; (c) 1000 mg/m² or 1250 mg/m² gemcitabine on days 1 and 8 of a 21-day cycle for 4 cycles and 5 or 6 AUC carboplatin on day 1 of the 21-day cycle for 4 cycles; (d) 500 mg/m² pemetrexed on day 1 of 21-day cycle for 4 cycles and 5 or 6 AUC carboplatin on day 1 of the 21-day cycle for 4 cycles, optionally wherein 500 mg/m² pemetrexed is administered every 4 weeks as a maintenance therapy after the 4 cycles; or (e) 500 mg/m² pemetrexed on day 1 of 21-day cycle for 4 cycles and 75 mg/m² cisplatin on day 1 of the 21-day cycle for 4 cycles, optionally wherein 500 mg/m² pemetrexed is administered every 4 weeks as a maintenance therapy after the 4 cycles.

82. The method of any one of claims 2, 24, and 32, wherein the tumor is a locally advanced, unresectable, stage III NSCLC tumor, and wherein (i) 1500 mg durvalumab or an antigen-binding fragment thereof is administered every 4 weeks and (ii) 3000 mg oleclumab or an antigen-binding fragment thereof is administered every 2 weeks for 2 months and then every 4 weeks.

83. The method of any one of claims 2, 24, and 32, wherein the tumor is a resectable, early NSCLC tumor, and wherein (i) 1500 mg durvalumab or an antigen-binding fragment thereof is administered and (ii) 3000 mg oleclumab or an antigen-binding fragment thereof is administered every 2 weeks.

84. The method of any one of claims 4, 24, 32, and 46 wherein the tumor is a metastatic microsatellite-stable colorectal cancer tumor, and wherein (i) 1500 mg durvalumab or an antigen-binding fragment thereof is administered every 4 weeks; (ii) 3000 mg oleclumab or an antigen-binding fragment thereof is administered every 2 weeks for four doses and then every 4 weeks; (iii) the chemotherapy comprises (a) 400 mg/m² of folinic acid every 2 weeks (b) 85 mg/m² oxaliplatin every 2 weeks; and (c) 2400 mg/m² of 5-fluorouracil every 2 weeks; and (iv) 5 mg/kg of bevacizumab or an antigen-binding fragment thereof is administered every 2 weeks.

85. The method of any one of claims 4, 24, and 32, wherein the tumor is a microsatellite-stable colorectal cancer tumor, and wherein (i) 1500 mg durvalumab or an antigen-binding fragment thereof is administered every 4 weeks; (ii) 3000 mg oleclumab or an antigen-binding fragment thereof is administered every 2 weeks for five doses and then every 4 weeks; and (iii) the chemotherapy comprises (a) 400 mg/m² of folinic acid every 2 weeks (b) 85 mg/m² oxaliplatin every 2 weeks; and (c) 400 mg/m² of 5-fluorouracil on day 1 and then 2400 mg/m² of 5-fluorouracil every 2 weeks.

86. The method of any one of claims 1-85, wherein the patient has an Eastern Cooperative Oncology Group (ECOG) performance status of 0 or 1.


SEQUENCE LISTING (24 sequences)

SEQ ID NO: 1 - protein, 110 aa, Artificial Sequence
  Light chain variable domain of oleclumab
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVL

SEQ ID NO: 2 - protein, 117 aa, Artificial Sequence
  Heavy chain variable domain of oleclumab
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSS

SEQ ID NO: 3 - protein, 5 aa, Artificial Sequence
  CDRH1 of oleclumab
SYAYS

SEQ ID NO: 4 - protein, 17 aa, Artificial Sequence
  CDRH2 of oleclumab
AISGSGGRTYYADSVKG

SEQ ID NO: 5 - protein, 8 aa, Artificial Sequence
  CDRH3 of oleclumab
LGYGRVDE

SEQ ID NO: 6 - protein, 13 aa, Artificial Sequence
  CDRL1 of oleclumab
SGSLSNIGRNPVN

SEQ ID NO: 7 - protein, 7 aa, Artificial Sequence
  CDRL2 of oleclumab
LDNLRLS

SEQ ID NO: 8 - protein, 11 aa, Artificial Sequence
  CDRL3 of oleclumab
ATWDDSHPGWT

SEQ ID NO: 9 - protein, 108 aa, Artificial Sequence
  Light chain variable domain of durvalumab
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 10 - protein, 121 aa, Artificial Sequence
  Heavy chain variable domain of durvalumab
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 11 - protein, 10 aa, Artificial Sequence
  CDRH1 of durvalumab
GFTFSRYWMS

SEQ ID NO: 12 - protein, 17 aa, Artificial Sequence
  CDRH2 of durvalumab
NIKQDGSEKYYVDSVKG

SEQ ID NO: 13 - protein, 12 aa, Artificial Sequence
  CDRH3 of durvalumab
EGGWFGELAFDY

SEQ ID NO: 14 - protein, 12 aa, Artificial Sequence
  CDRL1 of durvalumab
RASQRVSSSYLA

SEQ ID NO: 15 - protein, 7 aa, Artificial Sequence
  CDRL2 of durvalumab
DASSRAT

SEQ ID NO: 16 - protein, 9 aa, Artificial Sequence
  CDRL3 of durvalumab
QQYGSLPWT

SEQ ID NO: 17 - protein, 214 aa, Artificial Sequence
  Light chain of bevacizumab
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 453 aa, Artificial Sequence
  Heavy chain of bevacizumab
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 10 aa, Artificial Sequence
  CDRH1 of bevacizumab
GYTFTNYGMN

SEQ ID NO: 20 - protein, 17 aa, Artificial Sequence
  CDRH2 of bevacizumab
WINTYTGEPTYAADFKR

SEQ ID NO: 21 - protein, 14 aa, Artificial Sequence
  CDRH3 of bevacizumab
YPHYYGSSHWYFDV

SEQ ID NO: 22 - protein, 11 aa, Artificial Sequence
  CDRL1 of bevacizumab
SASQDISNYLN

SEQ ID NO: 23 - protein, 7 aa, Artificial Sequence
  CDRL2 of bevacizumab
FTSSLHS

SEQ ID NO: 24 - protein, 9 aa, Artificial Sequence
  CDRL3 of bevacizumab
QQYSTVPWT